SEQUENCE LISTING (10 sequences)

SEQ ID NO: 1 - DNA, 1338 bp, synthetic construct
  misc_feature 1..1338: note = Synthetic Polynucleotide
  source 1..1338: mol_type = other DNA
atggccgtcgcaatcgccgccgcaagagtgtggagactgaatcggggactgagccaggccgcactgctgctgctgagacagccaggagccagaggcctggccaggagccacccacctaggcagcagcagcagttcagctccctggacgataagccacagtttcccggcgcctctgccgagttcatcgacaagctggagtttatccagccaaacgtgatcagcggcatccccatctaccgcgtgatggaccggcagggccagatcatcaatccatccgaggacccccacctgccaaaggagaaggtgctgaagctgtacaagtctatgaccctgctgaacacaatggatagaatcctgtatgagtcccagcgccagggccggatctctttctacatgaccaactatggcgaggagggcacacacgtgggcagcgccgccgccctggacaataccgatctggtgttcggccagtatagagaggccggcgtgctgatgtacagggactatcctctggagctgtttatggcccagtgctacggcaatatcagcgatctgggcaagggccgccagatgccagtgcactatggctgtaaggagcggcacttcgtgaccatctctagccccctggccacacagatccctcaggcagtgggagcagcctacgccgccaagagagccaacgccaatagggtggtcatctgctattttggagagggagcagcctccgagggcgacgcacacgccggcttcaactttgccgccaccctggagtgccctatcatcttcttttgtagaaacaatggctacgccatctctaccccaacaagcgagcagtataggggcgatggaatcgcagccagaggcccaggctacggcatcatgtccatcagggtggacggcaacgacgtgttcgccgtgtataatgccacaaaggaggcacggagaagggcagtggcagagaaccagccctttctgatcgaggccatgacctacagaatcggccaccacagcacatccgacgattcctctgcctacaggtctgtggacgaagtgaattattgggacaagcaggatcaccctatcagcagactgaggcactatctgctgtcccagggctggtgggatgaggagcaggagaaggcctggaggaagcagagccgccggaaagtgatggaggccttcgagcaggcagagaggaagccaaagcccaaccctaatctgctgttttccgacgtgtaccaggagatgcctgcccagctgaggaagcagcaggagagcctggcaagacacctgcagacatacggcgagcattaccccctggaccattttgataagtga

SEQ ID NO: 2 - DNA, 6511 bp, synthetic construct
  misc_feature 1..6511: note = Synthetic Polynucleotide
  source 1..6511: mol_type = other DNA
cgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggccgtcgcaatcgccgccgcaagagtgtggagactgaatcggggactgagccaggccgcactgctgctgctgagacagccaggagccagaggcctggccaggagccacccacctaggcagcagcagcagttcagctccctggacgataagccacagtttcccggcgcctctgccgagttcatcgacaagctggagtttatccagccaaacgtgatcagcggcatccccatctaccgcgtgatggaccggcagggccagatcatcaatccatccgaggacccccacctgccaaaggagaaggtgctgaagctgtacaagtctatgaccctgctgaacacaatggatagaatcctgtatgagtcccagcgccagggccggatctctttctacatgaccaactatggcgaggagggcacacacgtgggcagcgccgccgccctggacaataccgatctggtgttcggccagtatagagaggccggcgtgctgatgtacagggactatcctctggagctgtttatggcccagtgctacggcaatatcagcgatctgggcaagggccgccagatgccagtgcactatggctgtaaggagcggcacttcgtgaccatctctagccccctggccacacagatccctcaggcagtgggagcagcctacgccgccaagagagccaacgccaatagggtggtcatctgctattttggagagggagcagcctccgagggcgacgcacacgccggcttcaactttgccgccaccctggagtgccctatcatcttcttttgtagaaacaatggctacgccatctctaccccaacaagcgagcagtataggggcgatggaatcgcagccagaggcccaggctacggcatcatgtccatcagggtggacggcaacgacgtgttcgccgtgtataatgccacaaaggaggcacggagaagggcagtggcagagaaccagccctttctgatcgaggccatgacctacagaatcggccaccacagcacatccgacgattcctctgcctacaggtctgtggacgaagtgaattattgggacaagcaggatcaccctatcagcagactgaggcactatctgctgtcccagggctggtgggatgaggagcaggagaaggcctggaggaagcagagccgccggaaagtgatggaggccttcgagcaggcagagaggaagccaaagcccaaccctaatctgctgttttccgacgtgtaccaggagatgcctgcccagctgaggaagcagcaggagagcctggcaagacacctgcagacatacggcgagcattaccccctggaccattttgataagtgaa

SEQ ID NO: 3 - DNA, 5282 bp, synthetic construct
  misc_feature 1..5282: note = Synthetic Polynucleotide
  source 1..5282: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggatatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgaggccaccatggccgtcgcaatcgccgccgcaagagtgtggagactgaatcggggactgagccaggccgcactgctgctgctgagacagccaggagccagaggcctggccaggagccacccacctaggcagcagcagcagttcagctccctggacgataagccacagtttcccggcgcctctgccgagttcatcgacaagctggagtttatccagccaaacgtgatcagcggcatccccatctaccgcgtgatggaccggcagggccagatcatcaatccatccgaggacccccacctgccaaaggagaaggtgctgaagctgtacaagtctatgaccctgctgaacacaatggatagaatcctgtatgagtcccagcgccagggccggatctctttctacatgaccaactatggcgaggagggcacacacgtgggcagcgccgccgccctggacaataccgatctggtgttcggccagtatagagaggccggcgtgctgatgtacagggactatcctctggagctgtttatggcccagtgctacggcaatatcagcgatctgggcaagggccgccagatgccagtgcactatggctgtaaggagcggcacttcgtgaccatctctagccccctggccacacagatccctcaggcagtgggagcagcctacgccgccaagagagccaacgccaatagggtggtcatctgctattttggagagggagcagcctccgagggcgacgcacacgccggcttcaactttgccgccaccctggagtgccctatcatcttcttttgtagaaacaatggctacgccatctctaccccaacaagcgagcagtataggggcgatggaatcgcagccagaggcccaggctacggcatcatgtccatcagggtggacggcaacgacgtgttcgccgtgtataatgccacaaaggaggcacggagaagggcagtggcagagaaccagccctttctgatcgaggccatgacctacagaatcggccaccacagcacatccgacgattcctctgcctacaggtctgtggacgaagtgaattattgggacaagcaggatcaccctatcagcagactgaggcactatctgctgtcccagggctggtgggatgaggagcaggagaaggcctggaggaagcagagccgccggaaagtgatggaggccttcgagcaggcagagaggaagccaaagcccaaccctaatctgctgttttccgacgtgtaccaggagatgcctgcccagctgaggaagcagcaggagagcctggcaagacacctgcagacatacggcgagcattaccccctggaccattttgataagtgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 4 - DNA, 1179 bp, synthetic construct
  misc_feature 1..1179: note = Synthetic Polynucleotide
  source 1..1179: mol_type = other DNA
atggccgtggtcgctgctgctgccggatggctgctgagactgcgggccgctggggctgagggacattggaggagactgcctggggctgggctggcaaggggcttcctgcaccctgcagcaacagtggaggacgcagcacagcggagacaggtggcccacttcacctttcagcccgatcctgagccacgcgagtacggccagacacagaagatgaacctgttccagtccgtgacctctgccctggacaatagcctggccaaggatccaacagccgtgatctttggcgaggacgtggccttcggcggcgtgtttcggtgcacagtgggcctgagagacaagtacggcaaggatcgggtgttcaacaccccactgtgcgagcagggaatcgtgggctttggcatcggcatcgcagtgaccggagcaacagcaatcgcagagatccagttcgccgactatatcttccccgcctttgatcagatcgtgaacgaggccgccaagtacaggtatcgctccggcgacctgtttaattgcggcagcctgaccatcagatccccttggggatgcgtgggacacggcgccctgtatcactctcagagcccagaggccttctttgcccactgccccggcatcaaggtggtcatcccacggagccccttccaggcaaagggcctgctgctgtcctgcatcgaggataagaacccctgtatcttctttgagcctaagatcctgtacagagcagcagcagaggaggtgcctatcgagccatataatatccctctgtctcaggccgaagtgatccaggagggaagcgacgtgaccctggtggcatggggaacacaggtgcacgtgatcagggaggtggcctccatggccaaggagaagctgggcgtgtcttgcgaagtgatcgatctgaggaccatcatcccttgggacgtggatacaatctgtaagtctgtgatcaagaccggccgcctgctgatcagccacgaggcaccactgacaggaggattcgcatccgagatcagctccaccgtgcaggaggagtgctttctgaatctggaggccccaatctctcgggtgtgcggctacgatacccccttccctcacatctttgagcctttctacatccctgacaagtggaagtgctacgacgctctgcggaagatgattaactattga

SEQ ID NO: 5 - DNA, 6352 bp, synthetic construct
  misc_feature 1..6352: note = Synthetic Polynucleotide
  source 1..6352: mol_type = other DNA
cgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggccgtggtcgctgctgctgccggatggctgctgagactgcgggccgctggggctgagggacattggaggagactgcctggggctgggctggcaaggggcttcctgcaccctgcagcaacagtggaggacgcagcacagcggagacaggtggcccacttcacctttcagcccgatcctgagccacgcgagtacggccagacacagaagatgaacctgttccagtccgtgacctctgccctggacaatagcctggccaaggatccaacagccgtgatctttggcgaggacgtggccttcggcggcgtgtttcggtgcacagtgggcctgagagacaagtacggcaaggatcgggtgttcaacaccccactgtgcgagcagggaatcgtgggctttggcatcggcatcgcagtgaccggagcaacagcaatcgcagagatccagttcgccgactatatcttccccgcctttgatcagatcgtgaacgaggccgccaagtacaggtatcgctccggcgacctgtttaattgcggcagcctgaccatcagatccccttggggatgcgtgggacacggcgccctgtatcactctcagagcccagaggccttctttgcccactgccccggcatcaaggtggtcatcccacggagccccttccaggcaaagggcctgctgctgtcctgcatcgaggataagaacccctgtatcttctttgagcctaagatcctgtacagagcagcagcagaggaggtgcctatcgagccatataatatccctctgtctcaggccgaagtgatccaggagggaagcgacgtgaccctggtggcatggggaacacaggtgcacgtgatcagggaggtggcctccatggccaaggagaagctgggcgtgtcttgcgaagtgatcgatctgaggaccatcatcccttgggacgtggatacaatctgtaagtctgtgatcaagaccggccgcctgctgatcagccacgaggcaccactgacaggaggattcgcatccgagatcagctccaccgtgcaggaggagtgctttctgaatctggaggccccaatctctcgggtgtgcggctacgatacccccttccctcacatctttgagcctttctacatccctgacaagtggaagtgctacgacgctctgcggaagatgattaactattgaa

SEQ ID NO: 6 - DNA, 5123 bp, synthetic construct
  misc_feature 1..5123: note = Synthetic Polynucleotide
  source 1..5123: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggatatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgaggccaccatggccgtggtcgctgctgctgccggatggctgctgagactgcgggccgctggggctgagggacattggaggagactgcctggggctgggctggcaaggggcttcctgcaccctgcagcaacagtggaggacgcagcacagcggagacaggtggcccacttcacctttcagcccgatcctgagccacgcgagtacggccagacacagaagatgaacctgttccagtccgtgacctctgccctggacaatagcctggccaaggatccaacagccgtgatctttggcgaggacgtggccttcggcggcgtgtttcggtgcacagtgggcctgagagacaagtacggcaaggatcgggtgttcaacaccccactgtgcgagcagggaatcgtgggctttggcatcggcatcgcagtgaccggagcaacagcaatcgcagagatccagttcgccgactatatcttccccgcctttgatcagatcgtgaacgaggccgccaagtacaggtatcgctccggcgacctgtttaattgcggcagcctgaccatcagatccccttggggatgcgtgggacacggcgccctgtatcactctcagagcccagaggccttctttgcccactgccccggcatcaaggtggtcatcccacggagccccttccaggcaaagggcctgctgctgtcctgcatcgaggataagaacccctgtatcttctttgagcctaagatcctgtacagagcagcagcagaggaggtgcctatcgagccatataatatccctctgtctcaggccgaagtgatccaggagggaagcgacgtgaccctggtggcatggggaacacaggtgcacgtgatcagggaggtggcctccatggccaaggagaagctgggcgtgtcttgcgaagtgatcgatctgaggaccatcatcccttgggacgtggatacaatctgtaagtctgtgatcaagaccggccgcctgctgatcagccacgaggcaccactgacaggaggattcgcatccgagatcagctccaccgtgcaggaggagtgctttctgaatctggaggccccaatctctcgggtgtgcggctacgatacccccttccctcacatctttgagcctttctacatccctgacaagtggaagtgctacgacgctctgcggaagatgattaactattgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 7 - DNA, 6782 bp, synthetic construct
  misc_feature 1..6782: note = Synthetic Polynucleotide
  source 1..6782: mol_type = other DNA
cgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacgccaccatggccgtggtcgctgctgctgccggatggctgctgagactgcgggccgctggggctgagggacattggaggagactgcctggggctgggctggcaaggggcttcctgcaccctgcagcaacagtggaggacgcagcacagcggagacaggtggcccacttcacctttcagcccgatcctgagccacgcgagtacggccagacacagaagatgaacctgttccagtccgtgacctctgccctggacaatagcctggccaaggatccaacagccgtgatctttggcgaggacgtggccttcggcggcgtgtttcggtgcacagtgggcctgagagacaagtacggcaaggatcgggtgttcaacaccccactgtgcgagcagggaatcgtgggctttggcatcggcatcgcagtgaccggagcaacagcaatcgcagagatccagttcgccgactatatcttccccgcctttgatcagatcgtgaacgaggccgccaagtacaggtatcgctccggcgacctgtttaattgcggcagcctgaccatcagatccccttggggatgcgtgggacacggcgccctgtatcactctcagagcccagaggccttctttgcccactgccccggcatcaaggtggtcatcccacggagccccttccaggcaaagggcctgctgctgtcctgcatcgaggataagaacccctgtatcttctttgagcctaagatcctgtacagagcagcagcagaggaggtgcctatcgagccatataatatccctctgtctcaggccgaagtgatccaggagggaagcgacgtgaccctggtggcatggggaacacaggtgcacgtgatcagggaggtggcctccatggccaaggagaagctgggcgtgtcttgcgaagtgatcgatctgaggaccatcatcccttgggacgtggatacaatctgtaagtctgtgatcaagaccggccgcctgctgatcagccacgaggcaccactgacaggaggattcgcatccgagatcagctccaccgtgcaggaggagtgctttctgaatctggaggccccaatctctcgggtgtgcggctacgatacccccttccctcacatctttgagcctttctacatccctgacaagtggaagtgctacgacgctctgcggaagatgattaactatggatccggtgagggcagaggaagtcttctaacatgcggtgacgtggaggagaatccgggccctgaattcgccaccatggccgtcgcaatcgccgccgcaagagtgtggagactgaatcggggactgagccaggccgcactgctgctgctgagacagccaggagccagaggcctggccaggagccacccacctaggcagcagcagcagttcagctccctggacgataagccacagtttcccggcgcctctgccgagttcatcgacaagctggagtttatccagccaaacgtgatcagcggcatccccatctaccgcgtgatggaccggcagggccagatcatcaatccatccgaggacccccacctgccaaaggagaaggtgctgaagctgtacaagtctatgaccctgctgaacacaatggatagaatcctgtatgagtcccagcgccagggccggatctctttctacatgaccaactatggcgaggagggcacacacgtgggcagcgccgccgccctggacaataccgatctggtgttcggccagtatagagaggccggcgtgctgatgtacagggactatcctctggagctgtttatggcccagtgctacggcaatatcagcgatctgggcaagggccgccagatgccagtgcactatggctgtaaggagcggcacttcgtgaccatctctagccccctggccacacagatccctcaggcagtgggagcagcctacgccgccaagagagccaacgccaatagggtggtcatctgctattttggagagggagcagcctccgagggcgacgcacacgccggcttcaactttgccgccaccctggagtgccctatcatcttcttttgtagaaacaatggctacgccatctctaccccaacaagcgagcagtataggggcgatggaatcgcagccagaggcccaggctacggcatcatgtccatcagggtggacggcaacgacgtgttcgccgtgtataatgccacaaaggaggcacggagaagggcagtggcagagaaccagccctttctgatcgaggccatgacctacagaatcggccaccacagcacatccgacgattcctctgcctacaggtctgtggacgaagtgaattattgggacaagcaggatcaccctatcagcagactgaggcactatctgctgtcccagggctggtgggatgaggagcaggagaaggcctggaggaagcagagccgccggaaagtgatggaggccttcgagcaggcagagaggaagccaaagcccaaccctaatctgctgttttccgacgtgtaccaggagatgcctgcccagctgaggaagcagcaggagagcctggcaagacacctgcagacatacggcgagcattaccccctggaccattttgataagtgaa

SEQ ID NO: 8 - DNA, 6971 bp, synthetic construct
  misc_feature 1..6971: note = Synthetic Polynucleotide
  source 1..6971: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcgatcacttatcaaaatggtccagggggtaatgctcgccgtatgtctgcaggtgtcttgccaggctctcctgctgcttcctcagctgggcaggcatctcctggtacacgtcggaaaacagcagattagggttgggctttggcttcctctctgcctgctcgaaggcctccatcactttccggcggctctgcttcctccaggccttctcctgctcctcatcccaccagccctgggacagcagatagtgcctcagtctgctgatagggtgatcctgcttgtcccaataattcacttcgtccacagacctgtaggcagaggaatcgtcggatgtgctgtggtggccgattctgtaggtcatggcctcgatcagaaagggctggttctctgccactgcccttctccgtgcctcctttgtggcattatacacggcgaacacgtcgttgccgtccaccctgatggacatgatgccgtagcctgggcctctggctgcgattccatcgcccctatactgctcgcttgttggggtagagatggcgtagccattgtttctacaaaagaagatgatagggcactccagggtggcggcaaagttgaagccggcgtgtgcgtcgccctcggaggctgctccctctccaaaatagcagatgaccaccctattggcgttggctctcttggcggcgtaggctgctcccactgcctgagggatctgtgtggccagggggctagagatggtcacgaagtgccgctccttacagccatagtgcactggcatctggcggcccttgcccagatcgctgatattgccgtagcactgggccataaacagctccagaggatagtccctgtacatcagcacgccggcctctctatactggccgaacaccagatcggtattgtccagggcggcggcgctgcccacgtgtgtgccctcctcgccatagttggtcatgtagaaagagatccggccctggcgctgggactcatacaggattctatccattgtgttcagcagggtcatagacttgtacagcttcagcaccttctcctttggcaggtgggggtcctcggatggattgatgatctggccctgccggtccatcacgcggtagatggggatgccgctgatcacgtttggctggataaactccagcttgtcgatgaactcggcagaggcgccgggaaactgtggcttatcgtccagggagctgaactgctgctgctgcctaggtgggtggctcctggccaggcctctggctcctggctgtctcagcagcagcagtgcggcctggctcagtccccgattcagtctccacactcttgcggcggcgattgcgacggccatggtggcctagcgctagagcttgctcccgcccgccgcgcgcttcgctttttatagggccgccgccgccgccgcctcgccataaaaggaaactttcggagcgcgccgctctgattggctgccgccgcacctctccgcctcgccccgccccgcccctcgcccccatcgctgcacaaaataattaaaaaataaataaatacaaaattgggggtggggaggggggggagatggggagagtgaagcagaacgtggcctcggatcccccgggctgcagtattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctcgctagcactagtgccaccatggccgtggtcgctgctgctgccggatggctgctgagactgcgggccgctggggctgagggacattggaggagactgcctggggctgggctggcaaggggcttcctgcaccctgcagcaacagtggaggacgcagcacagcggagacaggtggcccacttcacctttcagcccgatcctgagccacgcgagtacggccagacacagaagatgaacctgttccagtccgtgacctctgccctggacaatagcctggccaaggatccaacagccgtgatctttggcgaggacgtggccttcggcggcgtgtttcggtgcacagtgggcctgagagacaagtacggcaaggatcgggtgttcaacaccccactgtgcgagcagggaatcgtgggctttggcatcggcatcgcagtgaccggagcaacagcaatcgcagagatccagttcgccgactatatcttccccgcctttgatcagatcgtgaacgaggccgccaagtacaggtatcgctccggcgacctgtttaattgcggcagcctgaccatcagatccccttggggatgcgtgggacacggcgccctgtatcactctcagagcccagaggccttctttgcccactgccccggcatcaaggtggtcatcccacggagccccttccaggcaaagggcctgctgctgtcctgcatcgaggataagaacccctgtatcttctttgagcctaagatcctgtacagagcagcagcagaggaggtgcctatcgagccatataatatccctctgtctcaggccgaagtgatccaggagggaagcgacgtgaccctggtggcatggggaacacaggtgcacgtgatcagggaggtggcctccatggccaaggagaagctgggcgtgtcttgcgaagtgatcgatctgaggaccatcatcccttgggacgtggatacaatctgtaagtctgtgatcaagaccggccgcctgctgatcagccacgaggcaccactgacaggaggattcgcatccgagatcagctccaccgtgcaggaggagtgctttctgaatctggaggccccaatctctcgggtgtgcggctacgatacccccttccctcacatctttgagcctttctacatccctgacaagtggaagtgctacgacgctctgcggaagatgattaactattgagcggccgctctagagatctttttccctctgccaaaaattatggggacatcatgaagccccttgagcatctgacttctggctaataaaggaaatttattttcattgcaatagtgtgttggaattttttgtgtctctcactcggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtgggggggggggggggggg

SEQ ID NO: 9 - protein, 454 aa, Bos taurus
MQGSAKMAMAVAVAVARVWRPSRGLGRTGLPLLRLLGARGLARFHPHRWQQQQHFSSLDDKPQFPGASAEFIDKLEFIQPNVISGIPIYRVMDRQGQIINPSEDPHLPQEKVLKFYKSMTLLNTMDRILYESQRQGRISFYMTNYGEEGTHVGSAAALDDTDLVFGQYREAGVLMYRDYPLELFMAQCYGNVSDLGKGRQMPVHYGCRERHFVTISSPLATQIPQAVGAAYAAKRANANRWICYFGEGAASEGDAHAGFNFAATLECPIIFFCRNNGYAISTPTSEQYRGDGIAARGPGYGILSIRVDGNDVFAVYNATKEARRRAVAENQPFLIEAMTYRIGHHSTSDDSSAYRSVDEVNYWDKQDHPISRLRHHLQSRGWWDDEQEKAWRKQSRKKVMEAFEQAERKLKPNPSLIFSDVYQEMPAQLRKQQESLARHLQTYGEHYPLDHFEK

SEQ ID NO: 10 - protein, 444 aa, Homo sapiens
MAVAIAAARVWRLNRGLSQAALLLLRQPGARGLARSHPPRQQQQFSSLDDKPQFPGASAEFIDKLEFIQPNVISGIPIYRVMDRQGQIINPSEDPHLPKEKVLKLYKSMTLLNTMDRILYESQRQGRISFYMTNYGEEGTHVGSAAALDNTDLVFGQYREAGVLMYRDYPLELFMAQCYGNISDLGKGRQMPVHYGCKERHFVTISSPLATQIPQAVGAAYAAKRANANRWICYFGEGAASEGDAHAGFNFAATLECPIIFFCRNNGYAISTPTSEQYRGDGIAARGPGYGIMSIRVDGNDVFAVYNATKEARRRAVAENQPFLIEAMTYRIGHHSTSDDSSAYRSVDEVNYWDKQDHPISRLRHYLLSQGWWDEEQEKAWRKQSRRKVMEAFEQAERKPKPNPNLLFSDVYQEMPAQLRKQQESLARHLQTYGEHYPLDHFDK

(19) United States
(12) Patent Application Publication
(10) Pub. No.: US 2026/0265704 A1
(43) Pub. Date: Sep. 10, 2026

(54) AAV-MEDIATED GENE THERAPY FOR MAPLE SYRUP URINE DISEASE (MSUD)

(71) Applicants: University of Massachusetts, Westborough, MA (US); The Clinic for Special Children, Inc., Strasburg, PA (US)

(72) Inventors: Guangping Gao, Worcester, MA (US); Dan Wang, Worcester, MA (US); Jiaming Wang, Worcester, MA (US); Kevin A. Strauss, Strasburg, PA (US)

(73) Assignees: University of Massachusetts, Westborough, MA (US); The Clinic for Special Children, Inc., Strasburg, PA (US)

(21) Appl. No.: 19/681,769

(22) Filed: May 19, 2026

Related U.S. Application Data
(63) Continuation of application No. 19/283,401, filed on Jul. 29, 2025.
Application No. 19/283,401 is a division of application No. 17/602,353, filed on Apr. 10, 2020, now Pat. No. 12,398,378.
(62) Division of application No. 17/602,353, filed on Oct. 8, 2021, now Pat. No. 12,398,378.
(60) Provisional application No. 62/833,159, filed on Apr. 12, 2019.
(60) Provisional application No. 62/930,687, filed on Nov. 5, 2019.

(51) Int. Cl. C12N 9/02; A61K 48/00; A61P 3/00; C12N 15/86

(52) U.S. Cl. CPC C12N 9/0008; A61K 48/0058; A61P 3/00; C12N 15/86; C12Y 102/04004; C12N 2750/14143; C12N 2800/22

(57) ABSTRACT

In some aspects the disclosure provides compositions and methods for promoting expression of functional BCKDHA protein, which is the E1-alpha subunit of the branched-chain alpha-keto acid (BCAA) dehydrogenase complex, in a subject. In some aspects the disclosure provides compositions and methods for promoting expression of functional BCKDHB protein, which is the E1-beta subunit of the branched-chain alpha-keto acid (BCAA) dehydrogenase complex, in a subject. In some aspects the disclosure provides compositions and methods for promoting expression of functional BCKDHA and BCKDHB proteins, in a subject. In some embodiments, the disclosure provides methods of treating a subject having Maple Syrup Urine Disease (MSUD).

### CROSS-REFERENCE TO RELATED APPLICATIONS

This application is a continuation of U.S. application Ser. No. 19/283,401, filed Jul. 29, 2025, which is a division of U.S. application Ser. No. 17/602,353, filed Oct. 8, 2021, which is a national stage filing under 35 U.S.C. 371 of International Patent Application Serial No. PCT/US2020/027622, filed Apr. 10, 2020, which claims the benefit under 35 U.S.C. § 119 (e) of U.S. Application Ser. No. 62/930,687, filed Nov. 5, 2019, and U.S. Application Ser. No. 62/833,159, filed Apr. 12, 2019. This application is a division of U.S. application Ser. No. 17/602,353, filed Oct. 8, 2021. The entire contents of these applications are herein incorporated by reference in their entirety.

### REFERENCE TO AN ELECTRONIC SEQUENCE LISTING

The contents of the electronic sequence listing (U012070114US04-SEQ-KZM.xml; Size: 53,237 bytes; and Date of Creation: May 18, 2026) are herein incorporated by reference in their entirety.

### BACKGROUND OF INVENTION

Maple syrup urine disease (MSUD) is a rare genetic disorder affecting degradation of the branched-chain amino acids (BCAA; leucine, isoleucine, and valine) and their ketoacid derivatives. It is caused by biallelic mutations in one of three genes that encode subunits of the branched-chain ketoacid dehydrogenase complex (BCKDHA, BCKDHB, and DBT). Severe (i.e., classical) MSUD is fatal without treatment. Dietary BCAA restriction is the mainstay of treatment but is difficult to implement, has imperfect efficacy, and affords no protection against episodic and life-threatening encephalopathic crises. Liver transplantation is an effective alternative to dietary therapy, but entails risks of surgery and long-term immunosuppression.

### SUMMARY OF INVENTION

According to some aspects, the disclosure provides compositions and method for promoting expression of functional BCKDHA protein, which is the E1-alpha subunit of the branched-chain alpha-keto acid (BCAA) dehydrogenase complex, in a subject. In some embodiments, the methods involve administering to the subject an effective amount of a recombinant Adeno-associated virus (rAAV) comprising a capsid containing a nucleic acid engineered to express BCKDHA in the liver and/or skeletal muscle of a subject. In some embodiments, the subject comprises at least one endogenous BCKDHA allele having a loss-of-function mutation associated with MSUD. In some embodiments, the disclosure provides methods of treating a subject having MSUD that involve administering to the subject an effective amount of an rAAV comprising a capsid containing a nucleic acid engineered to express BCKDHA. In some embodiments, the nucleic acid expresses BCKDHA in the liver and/or skeletal muscle of the subject.

In some embodiments, the endogenous BCKDHA allele comprises a T-A transversion, resulting in a tyr394-to-asn (TYR394ASN). In some embodiments, the endogenous BCKDHA allele comprises a splice site mutation, a missense mutation, a truncation mutation or a nonsense mutation. In some embodiments, the endogenous BCKDHA alleles having the same loss-of-function mutations (homozygous state). In some embodiments, the endogenous BCKDHA alleles having different loss-of-function mutations (compound heterozygous state). In some embodiments, the endogenous BCKDHA allele comprises a 8-bp deletion (887_894del). In some embodiments, the endogenous BCKDHA allele comprises a 895G-A transition in exon 7, resulting in a gly245-to-arg (G245R) substitution. In some embodiments, the endogenous BCKDHA allele comprises a 1253T-G transversion, resulting in a phe364-to-cys (F364C) substitution. In some embodiments, the endogenous BCKDHA allele comprises a C-to-T transition resulting in an arg220-to-trp (R220W) substitution. In some embodiments, the endogenous BCKDHA allele comprises a G-to-A transition resulting in a gly204-to-ser (G204S) substitution. In some embodiments, the endogenous BCKDHA allele comprises a C-to-G transversion resulting in a thr265-to-arg (T265R) substitution. In some embodiments, the endogenous BCKDHA allele comprises a C-to-G transversion in the BCKDHA gene, resulting in a cys219-to-trp (C219W) substitution. In some embodiments, the endogenous BCKDHA allele comprises a 1-bp deletion (117delC), resulting in a frameshift, encoding a truncated protein with only 61 residues.

According to some aspects, the disclosure provides compositions and method for promoting expression of functional BCKDHB protein, which is the E1-beta subunit of the BCAA dehydrogenase complex, in a subject. In some embodiments, the methods involve administering to the subject an effective amount of an rAAV comprising a capsid containing a nucleic acid engineered to express BCKDHB in the liver and/or skeletal muscle of a subject. In some embodiments, the subject comprises at least one endogenous BCKDHB allele having a loss-of-function mutation associated with MSUD. In some embodiments, the disclosure provides methods of treating a subject having MSUD that involve administering to the subject an effective amount of an rAAV comprising a capsid containing a nucleic acid engineered to express BCKDHB. In some embodiments, the nucleic acid expresses BCKDHB in the liver and/or skeletal muscle of the subject.

In some embodiments, the endogenous BCKDHB allele comprises a splice site mutation, a missense mutation, a truncation mutation or a nonsense mutation. In some embodiments, the at least one endogenous BCKDHB allele comprises, an 11 base pair deletion in exon 1. In some embodiments, the at least one endogenous BCKDHB allele comprises, a guanine (G) to cytosine (C) change in exon 5, resulting in an arginine-to-proline substitution at residue 183 (R183P). In some embodiments, the at least one endogenous BCKDHB allele comprises, a C to thymine (T) transition, resulting in a histidine-to-tyrosine substitution at residue 156 (H156Y). In some embodiments, the at least one endogenous BCKDHB allele comprises, a T to G transversion, resulting in a valine-to-glycine substitution at residue 69 (V69G). In some embodiments, the at least one endogenous BCKDHB allele comprises, a 4 base pair deletion in intron 9 resulting in the deletion of exon 10, and an 8 base pair insertion in exon 10 resulting in a frameshift. In some embodiments, the at least one endogenous BCKDHB allele comprises, an 8 base pair insertion in exon 10.

According to some aspects, the disclosure provides compositions and method for promoting expression of functional BCKDHA and BCKDHB proteins, which are, respectively, the E1-alpha and E1-beta subunits of the BCAA dehydrogenase complex, in a subject. In some embodiments, the methods involve administering to the subject an effective amount of an rAAV comprising a capsid containing a nucleic acid engineered to express BCKDHA and BCKDHB in the liver and/or skeletal muscle of a subject. In some embodiments, the subject comprises at least one endogenous BCKDHA and/or BCKDHB allele having a loss-of-function mutation associated with MSUD. In some embodiments, the disclosure provides methods of treating a subject having MSUD that involve administering to the subject an effective amount of an rAAV comprising a capsid containing a nucleic acid engineered to express BCKDHA and BCKDHB. In some embodiments, the nucleic acid expresses BCKDHA and BCKDHB in the liver and/or skeletal muscle of the subject.

In some embodiments, the administration used in the methods described herein is systemic injection.

In some embodiments, the nucleic acids of the disclosure are engineered to express a codon-optimized human BCKDHA gene (opti-BCKDHA). In some embodiments, the nucleic acid comprises a sequence as set forth in any one of SEQ ID NO: 1-3. In some embodiments, the nucleic acids of the disclosure are engineered to express a codon-optimized human BCKDHB gene (opti-BCKDHB). In some embodiments, the nucleic acid comprises a sequence as set forth in any one of SEQ ID NO: 4-6. In some embodiments, the nucleic acids of the disclosure are engineered to express a codon-optimized human BCKDHA gene (opti-BCKDHA) and a codon-optimized human BCKDHB gene (opti-BCKDHB). In some embodiments, the nucleic acid comprises a sequence as set forth in any one of SEQ ID NO: 7-8.

In some embodiments, the nucleic acids of the disclosure comprise one or more adeno-associated virus (AAV) inverted terminal repeats (ITRs), wherein each ITR is selected from the group consisting of AAV1 ITR, AAV2 ITR, AAV3 ITR, AAV4 ITR, AAV5 ITR, and AAV6 ITR.

In some embodiments, the nucleic acid is a self-complementary AAV vector.

In an aspect, the disclosure relates to a method of treating a subject having Maple Syrup Urine Disease (MSUD), the method comprising administering to the subject an effective amount of an rAAV comprising a capsid containing a nucleic acid engineered to express BCKDHA in the liver and/or skeletal muscle of the subject.

In an aspect, the disclosure relates to an rAAV comprising an AAV9 capsid containing a nucleic acid engineered to express BCKDHA in the liver and/or skeletal muscle of the subject.

In some embodiments, the disclosure relates to a pharmaceutical composition comprising any of the rAAVs as disclosed herein.

In some embodiments, the disclosure relates to an isolated nucleic acid comprising a sequence as set forth by SEQ ID NO: 1 to 8.

In some embodiments, the disclosure relates to a host cell comprising any of the isolated nucleic acids disclosed herein. In some embodiments, the host cell is a eukaryotic cell.

In some embodiments, the disclosure relates to an isolated nucleic acid encoding an AAV capsid protein. In some embodiments, the capsid protein is AAV9 capsid protein.

These and other aspects and embodiments will be described in greater detail herein. The description of some exemplary embodiments of the disclosure are provided for illustration purposes only and not meant to be limiting. Additional compositions and methods are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, Drawings, Examples, and Claims.

### BRIEF DESCRIPTION OF THE DRAWINGS

FIGS. 1A-1C show AAV-constructs expressing BCKDHA and their expression in HEK 293T cells. FIG. 1A: Amino acids sequence alignment of human and bovine BCKDHA proteins. Arrowheads indicate mutations in the bovine model and Mennonite patients, respectively. Underline highlights signal peptide of BCKDHA protein. FIG. 1B: Cartoons showing two constructs expressing the same codon-optimized human BCKDHA complementary DNA (cDNA) in single-stranded and self-complementary forms, respectively. FIG. 1C: Western blot showing endogenous and AAV-borne BCKDHA protein in HEK293T cells.

FIGS. 2A-2B depict AAV-constructs expressing BCKDHA and BCKDHB. FIG. 2A: Schematics showing two constructs expressing the same codon-optimized human BCKDHB cDNA in single strand and self-complementary forms respectively. FIG. 2B: schematics showing two constructs expressing BCKDHA-BCKDHB cDNAs at the same time in single strand form. The upper panel using T2A sequence to link the two gene cassettes. The lower panel using bi-directional promoters sharing one common enhancer to drive the expression of two genes.

FIGS. 3A-3B depict AAV-constructs expression and activity test in HEK 293T cells. FIG. 3A: Western blot showing endogenous and AAV-constructs expressed BCKDHA and BCKDHB protein in wild-type (WT) and BCKDHA knock-out (KO) HEK 293T cells. BCKDHA vectors co-expressed with BCKDHB or alone are shown. BCKDHB could stabilize BCKDHA when co-expressed. FIG. 3B: BCKDC enzyme activity of WT and BCKDHA KO HEK 293T cells transfected with different AAV-constructs expressing BCKDHA or/and BCKDHB protein. BCKDC enzyme activity could be restored more efficiently when BCKDHA and BCKDHB were co-expressed in BCKDHA KO HEK 293T cells.

FIGS. 4A-4F show in vivo safety test of AAV-constructs expressing BCKDHA. FIG. 4A: Scheme of in vivo safety and efficiency test of AAV vectors delivery in WT mice. FIG. 4B: Injection dose of AAV vectors expressing BCKDHA. FIG. 4C: Male mice body weight curve with different doses injection. FIG. 4D: Female mice body weight curve with different doses injection. FIG. 4E: ALT test of mice with different doses injection. FIG. 4F: AST test of mice with different doses injection.

### DETAILED DESCRIPTION OF INVENTION

In some aspects, the disclosure relates to AAV-mediated gene replacement therapy for Maple Syrup Urine Disease (MSUD) caused by BCKDHA biallellic mutations. In some embodiments, AAV vectors are provided expressing a codon-optimized human BCKDHA gene (opti-BCKDHA), for which validated protein expression has been shown in various cell lines (e.g., Example 1). In some embodiments, an opti-BCKDHA cassette packaged into an rAAV comprising an AAV9 capsid. In some embodiments, the rAAV was delivered via systemic injection, and efficiently targets the liver and skeletal muscle, tissues which exhibit high levels of BCKDHA expression. In some embodiments, compared to these existing treatments, BCKDHA gene replacement therapy is as disclosed herein is a safer and more efficient option to treat MSUD.

In some embodiments, the endogenous BCKDHA allele comprises a T-A transversion, resulting in a tyr394-to-asn (TYR394ASN). In some embodiments, the endogenous BCKDHA allele comprises a splice site mutation, a missense mutation, a truncation mutation or a nonsense mutation. In some embodiments, the endogenous BCKDHA alleles having the same loss-of-function mutations (homozygous state). In some embodiments, the endogenous BCKDHA alleles having different loss-of-function mutations (compound heterozygous state). In some embodiments, the endogenous BCKDHA allele comprises a 8-bp deletion (887_894del). In some embodiments, the endogenous BCKDHA allele comprises a 895G-A transition in exon 7, resulting in a gly245-to-arg (G245R) substitution. In some embodiments, the endogenous BCKDHA allele comprises a 1253T-G transversion, resulting in a phe364-to-cys (F364C) substitution. In some embodiments, the endogenous BCKDHA allele comprises a C-to-T transition resulting in an arg220-to-trp (R220W) substitution. In some embodiments, the endogenous BCKDHA allele comprises a G-to-A transition resulting in a gly204-to-ser (G204S) substitution. In some embodiments, the endogenous BCKDHA allele comprises a C-to-G transversion resulting in a thr265-to-arg (T265R) substitution. In some embodiments, the endogenous BCKDHA allele comprises a C-to-G transversion in the BCKDHA gene, resulting in a cys219-to-trp (C219W) substitution. In some embodiments, the endogenous BCKDHA allele comprises a 1-bp deletion (117delC), resulting in a frameshift, encoding a truncated protein with only 61 residues.

In some aspects, the disclosure relates to AAV-mediated gene replacement therapy for MSUD caused by BCKDHB biallellic mutations. In some embodiments, AAV vectors are provided expressing a codon-optimized human BCKDHB gene (opti-BCKDHB), for which validated protein expression has been shown in various cell lines (e.g., Example 2). In some embodiments, an opti-BCKDHB cassette packaged into an rAAV comprising an AAV9 capsid. In some embodiments, the rAAV was delivered via systemic injection, and efficiently targets the liver and skeletal muscle, tissues which exhibit high levels of BCKDHB expression. In some embodiments, compared to these existing treatments, BCKDHB gene replacement therapy is as disclosed herein is a safer and more efficient option to treat MSUD.

In some embodiments, the endogenous BCKDHB allele comprises a splice site mutation, a missense mutation, a truncation mutation or a nonsense mutation. In some embodiments, the at least one endogenous BCKDHB allele comprises, an 11 base pair deletion in exon 1. In some embodiments, the at least one endogenous BCKDHB allele comprises, a guanine (G) to cytosine (C) change in exon 5, resulting in an arginine-to-proline substitution at residue 183 (R183P). In some embodiments, the at least one endogenous BCKDHB allele comprises, a C to thymine (T) transition, resulting in a histidine-to-tyrosine substitution at residue 156 (H156Y). In some embodiments, the at least one endogenous BCKDHB allele comprises, a T to G transversion, resulting in a valine-to-glycine substitution at residue 69 (V69G). In some embodiments, the at least one endogenous BCKDHB allele comprises, a 4 base pair deletion in intron 9 resulting in the deletion of exon 10, and an 8 base pair insertion in exon 10 resulting in a frameshift. In some embodiments, the at least one endogenous BCKDHB allele comprises, an 8 base pair insertion in exon 10.

In some aspects, the disclosure relates to AAV-mediated gene replacement therapy for MSUD caused by BCKDHA and/or BCKDHB biallellic mutations. In some embodiments, AAV vectors are provided expressing a codon-optimized human BCKDHA gene (opti-BCKDHA) and/or a codon-optimized human BCKDHB gene (opti-BCKDHB), for which validated protein expressions has been shown in various cell lines (e.g., Example 2). In some embodiments, an opti-BCKDHA and opti-BCKDHB cassette packaged into an rAAV comprising an AAV9 capsid. In some embodiments, the rAAV was delivered via systemic injection, and efficiently targets the liver and skeletal muscle, tissues which exhibit high levels of BCKDHA and/or BCKDHB expression. In some embodiments, compared to these existing treatments, the BCKDHA and BCKDHB gene replacement therapy, as disclosed herein, is a safer and more efficient option to treat MSUD.

### Isolated Nucleic Acids

In some aspects, the disclosure provides a nucleic acid comprising at least one transgene operably linked to a promoter, wherein the transgene encodes BCKDHA (branched chain keto acid dehydrogenase E1, alpha polypeptide; GENE ID: 593).

The BCKDHA gene encodes the E1-alpha subunit of the branched-chain alpha-keto acid (BCAA) dehydrogenase complex (BCKD; EC 1.2.4.4), an inner-mitochondrial enzyme complex that catalyzes the oxidative decarboxylation of the branched-chain alpha-ketoacids derived from isoleucine, leucine, and valine. This reaction is the second major step in the catabolism of the branched-chain amino acids. The BCKD complex consists of three catalytic components: a heterotetrameric (alpha2-beta2) branched-chain alpha-keto acid decarboxylase (E1), a homo-24-meric dihydrolipoyl transacylase (E2; 248610), and a homodimeric dihydrolipoamide dehydrogenase (E3; 238331). E1 is a thiamine pyrophosphate (TPP)-dependent enzyme. The reaction is irreversible and constitutes the first committed step in BCAA oxidation. The BCKDHB gene (248611) encodes the beta subunit of E1. The complex also contains 2 regulatory enzymes, a kinase and a phosphorylase.

The BCKDHA gene may encode an mRNA having the nucleotide sequence of NM_000709.4 or NM_001164783.1. The BCKDHA gene may encode a protein having the amino acid sequence NP_000700.1 or NP_001158255.1. In some embodiments, the opti-BCKDHA transgene comprises the sequence according to SEQ ID NO: 1. In some embodiments, the opti-BCKDHA transgene comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 1. The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category. Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms is incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.).

In some aspects, the disclosure provides a nucleic acid comprising at least one transgene operably linked to a promoter, wherein the transgene encodes BCKDHB (branched chain keto acid dehydrogenase E1, beta polypeptide; GENE ID: 594).

The BCKDHB gene encodes the E1-beta subunit of the branched-chain alpha-keto acid BCAA. The BCKDHB gene encodes the beta subunit of E1.

The BCKDHB gene may encode an mRNA having the nucleotide sequence of NM_000056.4, NM_001318975.1, or NM_183050.4. The BCKDHB gene may encode a protein having the amino acid sequence NP_000047.1, NP_898871.1, or NP_001305904.1. In some embodiments, the opti-BCKDHB transgene comprises the sequence according to SEQ ID NO: 4. In some embodiments, the opti-BCKDHB transgene comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 4.

A transgene which encodes a protein is generally operably linked to a promoter. As used herein, “operably linked” sequences include both expression control sequences that are contiguous with the gene of interest (e.g., transgene) and expression control sequences that act in trans or at a distance to control the gene of interest (e.g., transgene). In some embodiments, the promoter is a constitutive promoter, for example a chicken beta-actin (CBA) promoter, a retroviral Rous sarcoma virus (RSV) long terminal repeat (LTR) promoter (optionally with the RSV enhancer), a cytomegalovirus (CMV) promoter (optionally with the CMV enhancer) [see, e.g., Boshart et al., Cell, 41:521-530 (1985)], a Simian vacuolating virus 40 (SV40) promoter, a dihydrofolate reductase promoter, a beta-actin promoter, a phosphoglycerol kinase (PGK) promoter, and a EF1alpha promoter [Invitrogen]. In some embodiments, a promoter is an enhanced chicken beta-actin promoter. In some embodiments, a promoter is a U6 promoter.

In some embodiments, a promoter is an inducible promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene (e.g., BCKDHA, BCKDHB) will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the promoter drives transgene expression in neuronal tissues. In some embodiments, the disclosure provides a nucleic acid operably comprising a tissue-specific promoter operably linked to a transgene. As used herein, “tissue-specific promoter” refers to a promoter that preferentially regulates (e.g., drives or up-regulates) gene expression in a particular cell type relative to other cell types. A cell-type-specific promoter can be specific for any cell type, such as liver cells (e.g., hepatocytes), heart cells, muscle cells, etc.

Further examples of tissue-specific promoters include but are not limited to a liver-specific thyroxin binding globulin (TBG) promoter, an insulin promoter, a creatine kinase (MCK) promoter, an alpha-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998), and the immunoglobulin heavy chain promoter, among others which will be apparent to the skilled artisan.

In some aspects, the disclosure relates to isolated nucleic acids comprising a transgene (e.g., BCKDHA, BCKDHB) operably linked to a promoter via a chimeric intron. In some embodiments, a chimeric intron comprises a nucleic acid sequence from a chicken beta-actin gene, for example a non-coding intronic sequence from intron 1 of the chicken beta-actin gene. In some embodiments, the intronic sequence of the chicken beta-actin gene ranges from about 50 to about 150 nucleotides in length (e.g., any length between 50 and 150 nucleotides, inclusive). In some embodiments, the intronic sequence of the chicken beta-actin gene ranges from about 100 to 120 (e.g., 100, 101, 102, 103, 104, 105, 106, 107, 108, 109, 110, 111, 112, 113, 114, 115, 116, 117, 118, 119, or 120) nucleotides in length. In some embodiments, a chimeric intron is adjacent to one or more untranslated sequences (e.g., an untranslated sequence located between the promoter sequence and the chimeric intron sequence and/or an untranslated sequence located between the chimeric intron and the first codon of the transgene sequence). In some embodiments, each of the one or more untranslated sequences are non-coding sequences from a rabbit beta-globulin gene (e.g., untranslated sequence from rabbit beta-globulin exon 1, exon 2, etc.).

### Recombinant AAVs

The isolated nucleic acids of the disclosure may be recombinant Adeno-associated viral (rAAVs) vectors. In some embodiments, an isolated nucleic acid as described by the disclosure comprises a region (e.g., a first region) comprising a first adeno-associated virus (AAV) inverted terminal repeat (ITR), or a variant thereof and a second region comprising a first transgene encoding BCKDHA or BCKDHAB. In some embodiments, the second region comprises a first transgene encoding BCKDHA. In some embodiments, the second region comprises a first transgene encoding BCKDHB. In some embodiments, the isolated nucleic acid further comprises a third region comprising a second transgene, which may or may not be different than the first transgene. In some embodiments, the second region comprises a first transgene encoding BCKDHA and a third region comprises a second transgene encoding a second gene of interest (e.g., transgene). In some embodiments, the second region comprises a first transgene encoding BCKDHB and a third region comprises a second transgene encoding a second gene of interest (e.g., transgene). In some embodiments, the second region and third region each comprise the transgene encoding BCKDHA. In some embodiments, the second region and third region each comprise the transgene encoding BCKDHB. In some embodiments, the second region comprises a first transgene encoding BCKDHA and a third region comprises a second transgene encoding BCKDHB. In some embodiments, the second region comprises a first transgene encoding BCKDHB and a third region comprises a second transgene encoding BCKDHA. The isolated nucleic acid (e.g., the rAAV vector) may be packaged into a capsid protein and administered to a subject and/or delivered to a selected target cell. The transgene may also comprise a region encoding, for example, a protein and/or an expression control sequence (e.g., a poly-A tail), as described elsewhere in the disclosure.

The instant disclosure provides a vector comprising a single, cis-acting WT ITR. In some embodiments, the ITR is a 5′ITR. In some embodiments, the ITR is a 3′ ITR Generally, ITR sequences are about 145 bp in length. Preferably, substantially the entire sequences encoding the ITR(s) is used in the molecule, although some degree of minor modification of these sequences is permissible. In some embodiments, an ITR may be mutated at its terminal resolution site (TR), which inhibits replication at the vector terminus where the TR has been mutated and results in the formation of a self-complementary AAV. Another example of such a molecule employed in the present disclosure is a “cis-acting” plasmid containing the transgene, in which the selected transgene sequence and associated regulatory elements are flanked by the 5′ AAV ITR sequence and a 3′ hairpin-forming RNA sequence. Adeno-associated viral ITR sequences may be obtained from any known AAV, including presently identified mammalian AAV types. In some embodiments, an ITR sequence is an AAV2, AAV3, AAV4, AAV5, AAV6, AAV7, AAV8, AAVrh8, AAV9, AAV10, and/or AAVrh10 ITR sequence.

In some embodiments, an rAAV vector (e.g., pJW1-pAAV.pCB-CBA-opt-hBCKDHA-1) comprises a nucleic acid sequence according to SEQ ID NO: 2, or a portion thereof. In some embodiments, the rAAV vector comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 2.

In some embodiments, an rAAV vector is a self-complementary vector (e.g., pJW2-pAAVsc.CB6-opt-hBCKDHA-1) that comprises a nucleic acid sequence according to SEQ ID NO: 3, or a portion thereof. In some embodiments, the rAAV vector comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 3.

In some embodiments, an rAAV vector (e.g., pJW152-pAAV.pCB-CBA-opt-hBCKDHB) comprises a nucleic acid sequence according to SEQ ID NO: 5, or a portion thereof. In some embodiments, the rAAV vector comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 5.

In some embodiments, an rAAV vector is a self-complementary vector (e.g., pJW153-pAAVsc.CB6-opt-hBCKDHB) that comprises a nucleic acid sequence according to SEQ ID NO: 6, or a portion thereof. In some embodiments, the rAAV vector comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 62.

In some embodiments, an rAAV vector (e.g., pJW154-pAAV-pCB-opt-BCKDHB-T2A-opt-BCKDHA-1) comprises a nucleic acid sequence according to SEQ ID NO: 7, or a portion thereof. In some embodiments, the rAAV vector comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 7.

In some embodiments, an rAAV vector is a self-complementary vector (e.g., pJW162-SURE\Cell-pAAV-SV40-opti-BCKDHA-BiCB6-opti-BCKDHB-RBG) that comprises a nucleic acid sequence according to SEQ ID NO: 8, or a portion thereof. In some embodiments, the rAAV vector comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 8.

The isolated nucleic acids and/or rAAVs of the present disclosure may be modified and/or selected to enhance the targeting of the isolated nucleic acids and/or rAAVs to a target tissue (e.g., liver or skeletal muscle). Non-limiting methods of modifications and/or selections include AAV capsid serotypes (e.g., AAV9), tissue-specific promoters, and/or targeting peptides. In some embodiments, the isolated nucleic acids and rAAVs of the present disclosure comprise AAV capsid serotypes with enhanced targeting to liver or skeletal muscle tissues (e.g., AAV9). In some embodiments, the isolated nucleic acids and rAAVs of the present disclosure comprise tissue-specific promoters. In some embodiments, the isolated nucleic acids and rAAVs of the present disclosure comprise AAV capsid serotypes with enhanced targeting to liver or skeletal muscle tissues and tissue-specific promoters.

In some aspects, the disclosure provides isolated AAVs. As used herein with respect to AAVs, the term “isolated” refers to an AAV that has been artificially obtained or produced. Isolated AAVs may be produced using recombinant methods. Such AAVs are referred to herein as “recombinant AAVs” or “rAAVs.” Recombinant AAVs preferably have tissue-specific targeting capabilities, such that a transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected. In some embodiments, the rAAV comprises an AAV1, AAV2, AAV3, AAV4, AAV5, AAV6, AAV7, AAV8, AAVrh8, AAV9, AAV10 or AAVrh10 capsid protein, or a protein having substantial homology thereto. In some embodiments, the rAAV comprises an AAV9 capsid protein.

In some embodiments, the rAAVs of the disclosure are pseudo-typed rAAVs. Pseudo-typing is the process of producing viruses or viral vectors in combination with foreign viral envelope proteins. The result is a pseudo-typed virus particle. With this method, the foreign viral envelope proteins can be used to alter host tropism or an increased/decreased stability of the virus particles. In some aspects, a pseudo-typed rAAV comprises nucleic acids from two or more different AAVs, wherein the nucleic acid from one AAV encodes a capsid protein and the nucleic acid of at least one other AAV encodes other viral proteins and/or the viral genome. In some embodiments, a pseudo-typed rAAV refers to an AAV comprising an inverted terminal repeats (ITRs) of one AAV serotype and an capsid protein of a different AAV serotype. For example, a pseudo-typed AAV vector containing the ITRs of serotype X encapsidated with the proteins of Y will be designated as AAVX/Y (e.g., AAV2/1 has the ITRs of AAV2 and the capsid of AAV1). In some embodiments, pseudo-typed rAAVs may be useful for combining the tissue-specific targeting capabilities of a capsid protein from one AAV serotype with the viral DNA from another AAV serotype, thereby allowing targeted delivery of a transgene to a target tissue.

Methods for obtaining rAAVs having a desired capsid protein are well known in the art. (See, for example, US Patent Application Publication Number US 2003/0138772, the contents of which are incorporated herein by reference in their entirety). Typically the methods involve culturing a host cell which contains a nucleic acid sequence encoding an AAV capsid protein or fragment thereof; a functional rep gene; an rAAV vector composed of, AAV inverted terminal repeats (ITRs) and a transgene; and sufficient helper functions to permit packaging of the rAAV vector into the AAV capsid proteins. Typically, capsid proteins are structural proteins encoded by the cap gene of an AAV. In some embodiments, AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, capsid proteins protect a viral genome, deliver a genome and/or interact with a host cell. In some aspects, capsid proteins deliver the viral genome to a host in a tissue specific manner.

In some embodiments, the AAV capsid protein is of an AAV serotype selected from the group consisting of AAV3, AAV4, AAV5, AAV6, AAV8, AAVrh8 AAV9, AAV10 and AAVrh10. In some embodiments, the AAV capsid protein is of an AAVrh8 or AAVrh10 serotype. In some embodiments, the AAV capsid protein is of an AAVrh8 serotype.

In some embodiments, components to be cultured in the host cell to package an rAAV vector in an AAV capsid may be provided to the host cell in trans. Alternatively, any one or more of the required components (e.g., rAAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a coding sequence selected from the group consisting of: SEQ ID NO: 1-8 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 1-8 operably linked to a promoter. In some embodiments, the disclosure relates to a host cell containing a nucleic acid that comprises a coding sequence selected from the group consisting of: SEQ ID NO: 1 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 1 operably linked to a promoter. In some embodiments, the disclosure relates to a host cell containing a nucleic acid that comprises a coding sequence selected from the group consisting of: SEQ ID NO: 2 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 2 operably linked to a promoter. In some embodiments, the disclosure relates to a host cell containing a nucleic acid that comprises a coding sequence selected from the group consisting of: SEQ ID NO: 3 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 3 operably linked to a promoter. In some embodiments, the disclosure relates to a host cell containing a nucleic acid that comprises a coding sequence selected from the group consisting of: SEQ ID NO: 4 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 4 operably linked to a promoter. In some embodiments, the disclosure relates to a host cell containing a nucleic acid that comprises a coding sequence selected from the group consisting of: SEQ ID NO: 5 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 5 operably linked to a promoter. In some embodiments, the disclosure relates to a host cell containing a nucleic acid that comprises a coding sequence selected from the group consisting of: SEQ ID NO: 6 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 6 operably linked to a promoter. In some embodiments, the disclosure relates to a host cell containing a nucleic acid that comprises a coding sequence selected from the group consisting of: SEQ ID NO: 7 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 7 operably linked to a promoter. In some embodiments, the disclosure relates to a host cell containing a nucleic acid that comprises a coding sequence selected from the group consisting of: SEQ ID NO: 8 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 8 operably linked to a promoter.

In some embodiments, the disclosure relates to a composition comprising the host cell described above. In some embodiments, the composition comprising the host cell above further comprises a cryopreservative.

The rAAV vector, rep sequences, cap sequences, and helper functions useful for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions (i.e., infectious viral particle) are well known and the selection of a suitable method is not a limitation on the present disclosure. See, e.g., K. Fisher et al., J. Virol., 70:520-532 (1993) and U.S. Pat. No. 5,478,745.

In some embodiments, rAAVs may be produced using the triple transfection method (described in detail in U.S. Pat. No. 6,001,650). Typically, the rAAVs are produced by transfecting a host cell with an rAAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable WT AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced through the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of rAAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the term “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control,” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA, miRNA inhibitor) from a transcribed gene.

The foregoing methods for packaging recombinant vectors in desired AAV capsids to produce the rAAVs of the disclosure are not meant to be limiting and other suitable methods will be apparent to the skilled artisan.

### Recombinant AAV Vectors

The isolated nucleic acids of the present disclosure may be rAAV vectors. Recombinant AAV vectors of the disclosure are typically composed of, at a minimum, a transgene and its regulatory sequences, and 5′ and 3′ AAV ITRs. It is this rAAV vector which is packaged into a capsid protein and delivered to a selected target cell. In some embodiments, the transgene is a nucleic acid sequence, heterologous to the vector sequences, which encodes a polypeptide, protein, functional RNA molecule or other gene product, of interest. The nucleic acid coding sequence is operatively linked to regulatory components in a manner which permits transgene transcription, translation, and/or expression in a cell of a target tissue.

Aspects of the disclosure relate to the discovery that modifying the regulatory sequences of rAAVs provides levels of transgene expression that are therapeutically effective yet do not cause the vector-mediated toxicity associated with previously used rAAVs. Accordingly, in some embodiments, the disclosure relates to an rAAV comprising modified genetic regulatory elements. In some embodiments, the modified genetic regulatory element is a hybrid promoter.

As used herein, the term “hybrid promoter” refers to a regulatory construct capable of driving transcription an RNA transcript (e.g., a transcript comprising encoded by a transgene) in which the construct comprises two or more regulatory elements artificially arranged. Typically, a hybrid promoter comprises at least one element that is a minimal promoter and at least one element having an enhancer sequence or an intronic, exonic, or untranslated region (UTR) sequence comprising one or more transcriptional regulatory elements. In embodiments in which a hybrid promoter comprises an exonic, intronic, or UTR sequence, such sequence(s) may encode upstream portions of the RNA transcript while also containing regulatory elements that modulate (e.g., enhance) transcription of the transcript. In some embodiments, two or more elements of a hybrid promoter are from heterologous sources relative to one another. In some embodiments, two or more elements of a hybrid promoter are from heterologous sources relative to the transgene. In some embodiments, two or more elements of a hybrid promoter are from different genetic loci. In some embodiments, two or more elements of a hybrid promoter are from the same genetic locus but are arranged in a manner not found at the genetic locus. In some embodiments, the hybrid promoter comprise a first nucleic acid sequence from one promoter fused to one or more nucleic acid sequences comprises promoter or enhancer elements of a difference source. In some embodiments, a hybrid promoter comprises a first sequence from the chicken beta-actin promoter and a second sequence of the CMV enhancer. In some embodiments, a hybrid promoter comprises a first sequence from a chicken beta-actin promoter and a second sequence from an intron of a chicken-beta actin gene. In some embodiments, a hybrid promoter comprises a first sequence from the chicken beta-actin promoter fused to a CMV enhancer sequence and a sequence from an intron of the chicken-beta actin gene.

In some aspects, the rAAV comprises an enhancer element. As used herein, the term “enhancer element” refers to a nucleic acid sequence that when bound by an activator protein, activates or increases transcription of a gene or genes. Enhancer sequences can be upstream (i.e., 5′) or downstream (i.e., 3′) relative to the genes they regulate. Examples of enhancer sequences include cytomegalovirus (CMV) enhancer sequence and the SV40 enhancer sequence. In some embodiments, rAAVs comprise a CMV enhancer element or a portion thereof. As used herein, the term “a portion thereof” refers to a fragment of a nucleotide or amino acid sequence that retains the desired functional characteristic of the entire nucleotide or amino acid sequence from which it is derived. For example, a “CMV enhancer sequence or a portion thereof” refers to a nucleotide sequence derived from WT CMV enhancer that is capable of increasing transcription of a transgene.

In some aspects, the rAAV comprises a posttranscriptional response element. As used herein, the term “posttranscriptional response element” refers to a nucleic acid sequence that, when transcribed, adopts a tertiary structure that enhances expression of a gene. Examples of posttranscriptional regulatory elements include, but are not limited to, woodchuck hepatitis virus posttranscriptional regulatory element (WPRE), mouse RNA transport element (RTE), constitutive transport element (CTE) of the simian retrovirus type 1 (SRV-1), the CTE from the Mason-Pfizer monkey virus (MPMV), and the 5′ untranslated region of the human heat shock protein 70 (Hsp70 5′UTR). In some embodiments, the rAAV vector comprises a woodchuck hepatitis virus posttranscriptional regulatory element (WPRE).

In some aspects, the disclosure provides rAAV vectors comprising a hybrid or chimeric intron. As used herein, the term “chimeric intron” refers an intron having sequences from two or more different sources. In some embodiments, a chimeric intron comprises a nucleic acid encoding a splice donor site from a first source (e.g., organism or species) and a splice acceptor site from a second source (e.g., organism or species). In some embodiments, a chimeric intron comprise one or more transcriptional regulatory elements and/or enhancer sequences. In some embodiments, a chimeric intron is positioned between an exon of a hybrid promoter and transgene. In some embodiments, the disclosure provides an rAAV comprising a promoter operably linked to a transgene, wherein the transgene encodes a BCKDHA protein, and wherein the rAAV further comprises a chimeric intron. In some embodiments, the disclosure provides an rAAV comprising a promoter operably linked to a transgene, wherein the transgene encodes a BCKDHB protein, and wherein the rAAV further comprises a chimeric intron. In some embodiments, the disclosure provides an rAAV comprising a promoter operably linked to a first transgene, wherein the transgene encodes a BCKDHA protein and a second transgene wherein the transgene encodes a BCKDHB protein, and wherein the rAAV further comprises a chimeric intron.

In certain embodiments, the disclosure relates to rAAV vectors comprising artificial transcription elements. As used here, the term “artificial transcription element” refers, in some embodiments, to a synthetic sequence enabling the controlled transcription of DNA by an RNA polymerase to produce an RNA transcript. Transcriptionally active elements of the present disclosure are generally smaller than 500 bp, preferably smaller than 200 bp, more preferably smaller than 100, most preferably smaller than 50 bp. In some embodiments, an artificial transcription element comprises two or more nucleic acid sequences from transcriptionally active elements. Transcriptionally active elements are generally recognized in the art and include, for example, promoter, enhancer sequence, TATA box, G/C box, CCAAT box, specificity protein 1 (Sp1) binding site, Inr region, CRE (CAMP regulatory element), activating transcription factor 1 (ATF1) binding site, ATF1-CRE binding site, APBbeta box, APBalpha box, CArG box, CCAC box and those disclosed by U.S. Pat. No. 6,346,415. Combinations of the foregoing transcriptionally active elements are also contemplated.

In some embodiments, the artificial transcription element comprises promoter sequence. In some embodiments, the artificial transcription element comprises enhancer sequence. In some embodiments, the artificial transcription element comprises ATF1-CRE binding site. In some embodiments, the artificial transcription element comprises SP1 binding site. In some embodiments, the artificial transcription element comprises C box. In some embodiments, the artificial transcription element comprises TATA box. In some embodiments, the artificial transcription element comprises ATF1-CRE binding site, SP1 binding site and TATA box.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be “operably” linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA, miRNA inhibitor).

For nucleic acids encoding proteins, a polyadenylation sequence generally is inserted following the transgene sequences and before the 3′ AAV ITR sequence. An rAAV construct useful in the present disclosure may also contain an intron, desirably located between the promoter/enhancer sequence and the transgene. One possible intron sequence is derived from SV-40, and is referred to as the SV-40 T intron sequence.

Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459). The cleavage activity of the 2A sequence has previously been demonstrated in artificial systems including plasmids and gene therapy vectors (AAV and retroviruses) (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459; de Felipe, P et al., Gene Therapy, 1999; 6:198-208; de Felipe, P et al., Human Gene Therapy, 2000; 11:1921-1931.; and Klump, H et al., Gene Therapy, 2001; 8:811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the beta-actin promoter, the phosphoglycerol kinase (PGK) promoter, and the EF1alpha promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In some embodiments, the rAAVs of the disclosure are pseudo-typed rAAVs. For example, a pseudo-typed AAV vector containing the ITRs of serotype X encapsidated with the proteins of Y will be designated as AAVX/Y (e.g., AAV2/1 has the ITRs of AAV2 and the capsid of AAV1). In some embodiments, pseudo-typed rAAVs may be useful for combining the tissue-specific targeting capabilities of a capsid protein from one AAV serotype with the viral DNA from another AAV serotype, thereby allowing targeted delivery of a transgene to a target tissue.

In addition to the major elements identified above for the rAAV vector, the vector also includes conventional control elements necessary which are operably linked to the transgene in a manner which permits its transcription, translation and/or expression in a cell transfected with the plasmid vector or infected with the virus produced by the disclosure.

### Recombinant AAV Vector: Transgene Coding Sequences

The composition of the transgene sequence of the rAAV vector will depend upon the use to which the resulting vector will be put. For example, one type of transgene sequence includes a reporter sequence, which upon expression produces a detectable signal. In another example, the transgene encodes a therapeutic protein. In another example, the transgene encodes a protein that is intended to be used for research purposes, e.g., to create a somatic transgenic animal model harboring the transgene, e.g., to study the function of the transgene product. In another example, the transgene encodes a protein that is intended to be used to create an animal model of disease.

In some embodiments, the disclosure provides an rAAV comprising a transgene encoding BCKDHA. In some embodiments, the disclosure provides an rAAV comprising a transgene encoding BCKDHB. In some embodiments, the disclosure provides and rAAV comprising more than one transgene. In some embodiments, the rAAV comprises transgenes encoding BCKDHA and BCKDHB. Also contemplated herein are methods of treating MSUD by delivering a transgene to a subject using the rAAVs described herein. In some embodiments, the disclosure provides an rAAV comprising a transgene encoding BCKDHB. Also contemplated herein are methods of treating MSUD by delivering a transgene to a subject using the rAAVs described herein. Also contemplated herein are methods of treating MSUD by delivering a transgene to a subject using the rAAVs described herein. In some embodiments, the disclosure relates to a method for treating a MSUD, the method comprising administering an rAAV to a subject. In some embodiments, the rAAV comprises a hybrid promoter. In some embodiments, the rAAV comprises a chimeric intron. In some embodiments, the rAAV comprises an artificial transcription element. In some embodiments, the artificial transcription element comprises ATF1-CRE binding site, SP1 binding site and TATA box. In some embodiments, the promoter, chimeric intron or artificial transcription element is operably linked to a transgene. In some embodiments, the transgene encodes BCKDHA. In some embodiments, the transgene encodes BCKDHB. In some embodiments, transgenes encode BCKDHA and BCKDHB.

### Recombinant AAV Administration Methods

The rAAVs may be delivered to a subject in compositions according to any appropriate methods known in the art. The rAAV, preferably suspended in a physiologically compatible carrier (i.e., in a composition), may be administered to a subject (i.e., host animal (e.g., human, mouse, rat, cat, dog, sheep, rabbit, horse, cow, goat, pig, guinea pig, hamster, chicken, turkey, or a non-human primate (e.g., Macaque))). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue.

Aspects of the disclosure relate to compositions comprising an rAAV comprising at least one modified genetic regulatory sequence or element. In some embodiments, the composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

In some aspects, the disclosure relates to a composition (e.g., a pharmaceutical composition) comprising an rAAV comprising a nucleic acid encoding a BCKDHA. In some aspects, the disclosure relates to a composition (e.g., a pharmaceutical composition) comprising an rAAV comprising a nucleic acid encoding a BCKDHB. In some aspects, the disclosure relates to a composition (e.g., a pharmaceutical composition) comprising an rAAV comprising a nucleic acid encoding BCKDHA and BCKDHB.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

Recombinant AAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., injection into the liver, skeletal muscle), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine an rAAV virion dose range to treat a patient having a particular disease or disorder based on the aforementioned factors, as well as other factors that are well known in the art.

The terms “effective amount,” “therapeutically effective amount,” and “pharmaceutically effective amount,” as may be used interchangeably herein, refer to an amount of a biologically active agent (e.g., the isolated nucleic acids, rAAV, compositions of the present disclosure) sufficient to elicit a desired response. The effective amount will depend primarily on factors such as the species, age, weight, health of the subject, and the tissue to be targeted, and may thus vary among animal and tissue. For example, an effective amount of the rAAV is generally in the range from about 1 ml to about 100 ml of solution containing from about 10⁶ to 10¹⁶ genome copies (e.g., from 1×10⁶ to 1×10¹⁶, inclusive). In some cases, a dosage between about 10¹¹ to 10¹² rAAV genome copies is appropriate. In some embodiments, a dosage of between about 10¹¹ to 10¹³ rAAV genome copies is appropriate. In some embodiments, a dosage of between about 10¹¹ to 10¹⁴ rAAV genome copies is appropriate. In some embodiments, a dosage of between about 10¹¹ to 10¹⁵ rAAV genome copies is appropriate. In some embodiments, a dosage of 4.68×10⁷ is appropriate. In some embodiments, a dosage of 4.68×10⁸ genome copies is appropriate. In some embodiments, a dosage of 4.68×10⁹ genome copies is appropriate. In some embodiments, a dosage of 1.17×10¹⁰ genome copies is appropriate. In some embodiments, a dosage of 2.34×10¹⁰ genome copies is appropriate. In some embodiments, a dosage of 3.20×10¹¹ genome copies is appropriate. In some embodiments, a dosage of 1.2×10¹³ genome copies is appropriate. In some embodiments, a dosage of about 1×10¹⁴ vector genome (vg) copies is appropriate.

In some aspects, the disclosure relates to the recognition that one potential side-effect for administering an AAV to a subject is an immune response in the subject to the AAV, including inflammation. In some embodiments, a subject is immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, methotrexate, and any combination thereof.

In some embodiments, methods described by disclosure further comprise the step inducing immunosuppression (e.g., administering one or more immunosuppressive agents) in a subject prior to the subject being administered an rAAV (e.g., an rAAV or pharmaceutical composition as described by the disclosure). In some embodiments, a subject is immunosuppressed (e.g., immunosuppression is induced in the subject) between about 30 days and about 0 days (e.g., any time between 30 days until administration of the rAAV, inclusive) prior to administration of the rAAV to the subject. In some embodiments, the subject is pre-treated with immune suppression (e.g., rituximab, sirolimus, and/or prednisone) for at least 7 days.

In some embodiments, immunosuppression of a subject maintained during and/or after administration of an rAAV or pharmaceutical composition. In some embodiments, a subject is immunosuppressed (e.g., administered one or more immunosuppressants) for between 1 day and 1 year after administration of the rAAV or pharmaceutical composition.

In some embodiments, rAAV compositions are formulated to reduce aggregation of AAV particles in the composition, particularly where high rAAV concentrations are present (e.g., ~10¹³ GC/ml or more). Methods for reducing aggregation of rAAVs are well known in the art and, include, for example, addition of surfactants, pH adjustment, salt concentration adjustment, etc. See, e.g., Wright F R, et al., Molecular Therapy (2005) 12, 171-178, the contents of which are incorporated herein by reference.

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intrapancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515; and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Angstroms, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the disclosure relates to administration of one or more additional therapeutic agents to a subject who has been administered an rAAV or pharmaceutical composition as described herein.

### Kits and Related Compositions

The agents (e.g., nucleic acids, rAAV, vectors, etc.) described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the disclosure relates to a kit for producing an rAAV, the kit comprising a container housing an isolated nucleic acid having a sequence of any one of SEQ ID NO: 1-8. In some embodiments, the kit further comprises instructions for producing the rAAV. In some embodiments, the kit further comprises at least one container housing an rAAV vector, wherein the rAAV vector comprises a transgene.

In some embodiments, the disclosure relates to a kit comprising a container housing an rAAV as described supra. In some embodiments, the kit further comprises a container housing a pharmaceutically acceptable carrier. For example, a kit may comprise one container housing an rAAV and a second container housing a buffer suitable for injection of the rAAV into a subject. In some embodiments, the container is a syringe.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or intravenous (iv) needle tubing and bag, particularly in the case of the kits for producing specific somatic animal models.

In some cases, the methods involve transfecting cells with total cellular DNAs isolated from the tissues that potentially harbor proviral AAV genomes at very low abundance and supplementing with helper virus function (e.g., adenovirus) to trigger and/or boost AAV rep and cap gene transcription in the transfected cell. In some cases, RNA from the transfected cells provides a template for RT-PCR amplification of cDNA and the detection of novel AAVs. In cases where cells are transfected with total cellular DNAs isolated from the tissues that potentially harbor proviral AAV genomes, it is often desirable to supplement the cells with factors that promote AAV gene transcription. For example, the cells may also be infected with a helper virus, such as an Adenovirus or a Herpes Virus. In a specific embodiment, the helper functions are provided by an adenovirus. The adenovirus may be a WT adenovirus, and may be of human or non-human origin, preferably non-human primate (NHP) origin. Similarly adenoviruses known to infect non-human animals (e.g., chimpanzees, mouse) may also be employed in the methods of the disclosure (See, e.g., U.S. Pat. No. 6,083,716). In addition to WT adenoviruses, recombinant viruses or non-viral vectors (e.g., plasmids, episomes, etc.) carrying the necessary helper functions may be utilized. Such recombinant viruses are known in the art and may be prepared according to published techniques. See, e.g., U.S. Pat. Nos. 5,871,982 and 6,251,677, which describe a hybrid Ad/AAV virus. A variety of adenovirus strains are available from the American Type Culture Collection, Manassas, Va., or available by request from a variety of commercial and institutional sources. Further, the sequences of many such strains are available from a variety of databases including, e.g., PubMed and GenBank.

Cells may also be transfected with a vector (e.g., helper vector) which provides helper functions to the AAV. The vector providing helper functions may provide adenovirus functions, including, e.g., E1a, E1b, E2a, E4ORF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

In some cases, a novel isolated capsid gene can be used to construct and package rAAV vectors, using methods well known in the art, to determine functional characteristics associated with the novel capsid protein encoded by the gene. For example, novel isolated capsid genes can be used to construct and package rAAV vectors comprising a reporter gene (e.g., B-Galactosidase, GFP, Luciferase, etc.). The rAAV vector can then be delivered to an animal (e.g., mouse) and the tissue targeting properties of the novel isolated capsid gene can be determined by examining the expression of the reporter gene in various tissues (e.g., heart, liver, kidneys) of the animal. Other methods for characterizing the novel isolated capsid genes are disclosed herein and still others are well known in the art.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for detecting a latent AAV in a cell. In addition, kits of the disclosure may include, instructions, a negative and/or positive control, containers, diluents and buffers for the sample, sample preparation tubes and a printed or electronic table of reference AAV sequence for sequence comparisons.

### Methods of Treating Maple Syrup Urine Disease (MSUD)

Aspects of the present disclosure provide methods for treating a MSUD. MSUD caused by: a mutation in the E1-alpha subunit gene is referred to as MSUD “type IA;” caused by a mutation in the E1-beta subunit gene as “type IB;” and that caused by defect in the E2 subunit gene as “type II.” In some embodiments, clinical features of MSUD are mental and physical retardation, feeding problems, and a maple syrup odor to the urine. In some embodiments, keto acids of branched-chain amino acids are present in urine, resulting from a block in oxidative decarboxylation.

Accordingly, in some embodiments, the disclosure provides isolated nucleic acids, rAAVs, compositions, and methods useful in treating MSUD. The terms “treatment,” “treat,” and “treating,” as may be used interchangeably herein, refer to a clinical intervention aimed to reverse, alleviate, delay the onset of, or inhibit the progress of a, indication, disease, disorder, or one or more symptoms thereof, as described herein (e.g., MSUD). In some embodiments, treatment may be administered after one or more symptoms have developed and/or after a disease has been diagnosed. In other embodiments, treatment may be administered in the absence of symptoms (e.g., to prevent or delay onset of a symptom or inhibit onset or progression of a disease). For example, treatment may be administered to a susceptible individual (e.g., subject) prior to the onset of symptoms (e.g., in light of a history of symptoms and/or in light of genetic or other susceptibility factors). Treatment may also be continued after symptoms have resolved, for example, to prevent or delay their recurrence.

In some aspects, the disclosure relates to a method for promoting expression of functional BCKDHA protein, which is the E1-alpha subunit of the branched-chain alpha-keto acid (BCAA) dehydrogenase complex, in a subject, the method comprising administering to the subject an effective amount of an rAAV comprising a capsid containing a nucleic acid engineered to express BCKDHA in the liver and/or skeletal muscle of the subject, wherein the subject comprises at least one endogenous BCKDHA allele having a loss-of-function mutation associated with Maple Syrup Urine Disease (MSUD). In some embodiments, the isolated nucleic acids, rAAVs, compositions, and methods are for the treatment of MSUD. In some embodiments, MSUD in a subject may be the result of at least one endogenous BCKDHA allele having a loss-of-function mutation associated with Maple Syrup Urine Disease (MSUD).

In some embodiments, the at least one endogenous BCKDHA allele comprises a T-A transversion, resulting in a tyr394-to-asn (TYR394ASN). In some embodiments, the at least one endogenous BCKDHA allele comprises a splice site mutation, a missense mutation, a truncation mutation, or a nonsense mutation. In some embodiments, the endogenous BCKDHA allele comprises a 8 base pair deletion (887_894del). In some embodiments, the endogenous BCKDHA allele comprises a 895G-A transition in exon 7, resulting in a gly245-to-arg (G245R) substitution. In some embodiments, the endogenous BCKDHA allele comprises a 1253T-G transversion, resulting in a phe364-to-cys (F364C) substitution. In some embodiments, the endogenous BCKDHA allele comprises a C-to-T transition resulting in an arg220-to-trp (R220W) substitution. In some embodiments, the endogenous BCKDHA allele comprises a G-to-A transition resulting in a gly204-to-ser (G204S) substitution. In some embodiments, the endogenous BCKDHA allele comprises a C-to-G transversion resulting in a thr265-to-arg (T265R) substitution. In some embodiments, the endogenous BCKDHA allele comprises a C-to-G transversion in the BCKDHA gene, resulting in a cys219-to-trp (C219W) substitution. In some embodiments, the endogenous BCKDHA allele comprises a 1 base pair deletion (117delC), resulting in a frameshift, encoding a truncated protein with only 61 residues.

In some embodiments, the subject has two endogenous BCKDHA alleles having the same loss-of-function mutations (homozygous state). In some embodiments, the subject has two endogenous BCKDHA alleles having different loss-of-function mutations (compound heterozygous state).

A method for promoting expression of functional BCKDHB protein, which is the E1-beta subunit of the branched-chain alpha-keto acid (BCAA) dehydrogenase complex, in a subject, the method comprising administering to the subject an effective amount of an rAAV comprising a capsid containing a nucleic acid engineered to express BCKDHB in the liver and/or skeletal muscle of the subject, wherein the subject comprises at least one endogenous BCKDHB allele having a loss-of-function mutation associated with Maple Syrup Urine Disease (MSUD). In some embodiments, MSUD in a subject may be the result of at least one endogenous BCKDHB allele having at least one endogenous BCKDHB allele having a loss-of-function mutation associated with Maple Syrup Urine Disease (MSUD).

In some embodiments, the at least one endogenous BCKDHB allele comprises, an 11 base pair deletion in exon 1. In some embodiments, the at least one endogenous BCKDHB allele comprises, a guanine (G) to cytosine (C) change in exon 5, resulting in an arginine-to-proline substitution at residue 183 (R183P). In some embodiments, the at least one endogenous BCKDHB allele comprises, a C to thymine (T) transition, resulting in a histidine-to-tyrosine substitution at residue 156 (H156Y). In some embodiments, the at least one endogenous BCKDHB allele comprises, a T to G transversion, resulting in a valine-to-glycine substitution at residue 69 (V69G). In some embodiments, the at least one endogenous BCKDHB allele comprises, a 4 base pair deletion in intron 9 resulting in the deletion of exon 10, and an 8 base pair insertion in exon 10 resulting in a frameshift. In some embodiments, the at least one endogenous BCKDHB allele comprises, an 8 base pair insertion in exon 10. In some embodiments, the at least one endogenous BCKDHB allele comprises a splice site mutation, a missense mutation, a truncation mutation, or a nonsense mutation. In some embodiments, the subject has two endogenous BCKDHB alleles having the same loss-of-function mutations (homozygous state). In some embodiments, the subject has two endogenous BCKDHB alleles having different loss-of-function mutations (compound heterozygous state).

Methods for treating MSUD in a subject may comprise administering an isolated nucleic acid, rAAV, or composition of the present disclosure that comprises a transgene encoding BCKDHA. In some embodiments, the method (e.g., administration of the isolated nucleic acid, rAAV, or compositions of the present disclosure) increases the expression of functional BCKDKA in a subject. In some embodiments, the method (e.g., administration of the isolated nucleic acid, rAAV, or compositions of the present disclosure) increases the functional expression of BCKDKA at least 10%, at least 20%, at least 30%, at least 40%, at least 50%, at least 60%, at least 70%, at least 80%, at least 90% or more, in a subject (e.g., relative to the subject prior to administration of the rAAV). In some embodiments, the method (e.g., administration of the isolated nucleic acid, rAAV, or compositions of the present disclosure) increases the degradation of the branched-chain amino acids (BCAA; leucine, isoleucine, and valine) and their ketoacid derivatives. In some embodiments, the method (e.g., administration of the isolated nucleic acid, rAAV, or compositions of the present disclosure) increases the degradation of the branched-chain amino acids (BCAA; leucine, isoleucine, and valine) and their ketoacid derivatives at least 10%, at least 20%, at least 30%, at least 40%, at least 50%, at least 60%, at least 70%, at least 80%, at least 90% or more, in a subject (e.g., relative to the subject prior to administration of the rAAV).

Methods for treating MSUD in a subject may comprise administering an isolated nucleic acid, rAAV, or composition of the present disclosure that comprises a transgene encoding BCKDHB. In some embodiments, the method (e.g., administration of the isolated nucleic acid, rAAV, or compositions of the present disclosure) increases the expression of functional BCKDKB in a subject. In some embodiments, the method (e.g., administration of the isolated nucleic acid, rAAV, or compositions of the present disclosure) increases the functional expression of BCKDKB at least 10%, at least 20%, at least 30%, at least 40%, at least 50%, at least 60%, at least 70%, at least 80%, at least 90% or more, in a subject (e.g., relative to the subject prior to administration of the rAAV). In some embodiments, the method (e.g., administration of the isolated nucleic acid, rAAV, or compositions of the present disclosure) increases the degradation of the branched-chain amino acids (BCAA; leucine, isoleucine, and valine) and their ketoacid derivatives (e.g., relative to the subject prior to administration of the rAAV). In some embodiments, the method (e.g., administration of the isolated nucleic acid, rAAV, or compositions of the present disclosure) increases the degradation of the branched-chain amino acids (BCAA; leucine, isoleucine, and valine) and their ketoacid derivatives at least 10%, at least 20%, at least 30%, at least 40%, at least 50%, at least 60%, at least 70%, at least 80%, at least 90% or more, in a subject (e.g., relative to the subject prior to administration of the rAAV).

In some embodiments, the method (e.g., administration of the isolated nucleic acid, rAAV, or compositions of the present disclosure) reduce the ratio of leucine to alanine. In some embodiments, the method (e.g., administration of the isolated nucleic acid, rAAV, or compositions of the present disclosure) reduce the ratio of leucine to alanine by at least 10%, at least 20%, at least 30%, at least 40%, at least 50%, at least 60%, at least 70%, at least 80%, at least 90% or more, in a subject (e.g., relative to the subject prior to administration of the rAAV). In some embodiments, the method (e.g., administration of the isolated nucleic acid, rAAV, or compositions of the present disclosure) reduce the ratio of leucine to alanine to 0.4 or less. In some embodiments, the method (e.g., administration of the isolated nucleic acid, rAAV, or compositions of the present disclosure) reduce urine branched-chain α-ketoacids (BCKA): 2-oxoisocaproate, 2-oxo-3-methylvalerate, 2-oxoisovalerate, 2-hydroxyisovalerate, 2-hydroxyisocaproate, and/or 2-hydroxy-3-methylvalerate.

A subject may be a human, a mouse, a rat, a pig, a dog, a cat, cattle, or a non-human primate. In some embodiments, the subject is a human. Administering means contacting a cell or subject with an isolated nucleic acid, rAAV, or composition of the present disclosure. Non-limiting examples of administering including intravenous injection, intraarterial injection, intracranial injection, intrathecal injection, intracerebral injection, infusion, or inhalation.

### Exemplary Sequences

This Table exhibits some exemplary sequences as disclosed by the instant Specification, but is not limiting. This Specification includes a Sequence Listing submitted concurrently herewith as a text file in ASCII format. The Sequence Listing and all of the information contained therein are expressly incorporated herein and constitute part of the instant Specification as filed.

TABLE 1
Exemplary Sequences

| SEQ | | |
|---|---|---|
| ID | | |
| NO. | Sequence* | Description |
| 1 | ATGGCCGTCGCAATCGCCGCCGCAAGAGTGTGGAGACTGAATCGGGGACTG | opti- |
| | AGCCAGGCCGCACTGCTGCTGCTGAGACAGCCAGGAGCCAGAGGCCTGGCC | BCKDHA |
| | AGGAGCCACCCACCTAGGCAGCAGCAGCAGTTCAGCTCCCTGGACGATAAG | |
| | CCACAGTTTCCCGGCGCCTCTGCCGAGTTCATCGACAAGCTGGAGTTTATC | |
| | CAGCCAAACGTGATCAGCGGCATCCCCATCTACCGCGTGATGGACCGGCAG | |
| | GGCCAGATCATCAATCCATCCGAGGACCCCCACCTGCCAAAGGAGAAGGTG | |
| | CTGAAGCTGTACAAGTCTATGACCCTGCTGAACACAATGGATAGAATCCTG | |
| | TATGAGTCCCAGCGCCAGGGCCGGATCTCTTTCTACATGACCAACTATGGC | |
| | GAGGAGGGCACACACGTGGGCAGCGCCGCCGCCCTGGACAATACCGATCTG | |
| | GTGTTCGGCCAGTATAGAGAGGCCGGCGTGCTGATGTACAGGGACTATCCT | |
| | CTGGAGCTGTTTATGGCCCAGTGCTACGGCAATATCAGCGATCTGGGCAAG | |
| | GGCCGCCAGATGCCAGTGCACTATGGCTGTAAGGAGCGGCACTTCGTGACC | |
| | ATCTCTAGCCCCCTGGCCACACAGATCCCTCAGGCAGTGGGAGCAGCCTAC | |
| | GCCGCCAAGAGAGCCAACGCCAATAGGGTGGTCATCTGCTATTTTGGAGAG | |
| | GGAGCAGCCTCCGAGGGCGACGCACACGCCGGCTTCAACTTTGCCGCCACC | |
| | CTGGAGTGCCCTATCATCTTCTTTTGTAGAAACAATGGCTACGCCATCTCT | |
| | ACCCCAACAAGCGAGCAGTATAGGGGCGATGGAATCGCAGCCAGAGGCCCA | |
| | GGCTACGGCATCATGTCCATCAGGGTGGACGGCAACGACGTGTTCGCCGTG | |
| | TATAATGCCACAAAGGAGGCACGGAGAAGGGCAGTGGCAGAGAACCAGCCC | |
| | TTTCTGATCGAGGCCATGACCTACAGAATCGGCCACCACAGCACATCCGAC | |
| | GATTCCTCTGCCTACAGGTCTGTGGACGAAGTGAATTATTGGGACAAGCAG | |
| | GATCACCCTATCAGCAGACTGAGGCACTATCTGCTGTCCCAGGGCTGGTGG | |
| | GATGAGGAGCAGGAGAAGGCCTGGAGGAAGCAGAGCCGCCGGAAAGTGATG | |
| | GAGGCCTTCGAGCAGGCAGAGAGGAAGCCAAAGCCCAACCCTAATCTGCTG | |
| | TTTTCCGACGTGTACCAGGAGATGCCTGCCCAGCTGAGGAAGCAGCAGGAG | |
| | AGCCTGGCAAGACACCTGCAGACATACGGCGAGCATTACCCCCTGGACCAT | |
| | TTTGATAAGTGA | |
| 2 | CGCGTGGTACCTCTAGAGTCGACCCGGGCGGCCTCGAGGACGGGGTGAACT | rAAV |
| | ACGCCTGAGGATCCGATCTTTTTCCCTCTGCCAAAAATTATGGGGACATCA | vector: |
| | TGAAGCCCCTTGAGCATCTGACTTCTGGCTAATAAAGGAAATTTATTTTCA | pJW1- |
| | TTGCAATAGTGTGTTGGAATTTTTTGTGTCTCTCACTCGGAAGCAATTCGT | pAAV.pCB- |
| | TGATCTGAATTTCGACCACCCATAATACCCATTACCCTGGTAGATAAGTAG | CBA-opt- |
| | CATGGCGGGTTAATCATTAACTACAAGGAACCCCTAGTGATGGAGTTGGCC | hBCKDHA-1 |
| | ACTCCCTCTCTGCGCGCTCGCTCGCTCACTGAGGCCGGGCGACCAAAGGTC | |
| | GCCCGACGCCCGGGCTTTGCCCGGGCGGCCTCAGTGAGCGAGCGAGCGCGC | |
| | AGCCTTAATTAACCTAATTCACTGGCCGTCGTTTTACAACGTCGTGACTGG | |
| | GAAAACCCTGGCGTTACCCAACTTAATCGCCTTGCAGCACATCCCCCTTTC | |
| | GCCAGCTGGCGTAATAGCGAAGAGGCCCGCACCGATCGCCCTTCCCAACAG | |
| | TTGCGCAGCCTGAATGGCGAATGGGACGCGCCCTGTAGCGGCGCATTAAGC | |
| | GCGGCGGGTGTGGTGGTTACGCGCAGCGTGACCGCTACACTTGCCAGCGCC | |
| | CTAGCGCCCGCTCCTTTCGCTTTCTTCCCTTCCTTTCTCGCCACGTTCGCC | |
| | GGCTTTCCCCGTCAAGCTCTAAATCGGGGGCTCCCTTTAGGGTTCCGATTT | |
| | AGTGCTTTACGGCACCTCGACCCCAAAAAACTTGATTAGGGTGATGGTTCA | |
| | CGTAGTGGGCCATCGCCCTGATAGACGGTTTTTCGCCCTTTGACGTTGGAG | |
| | TCCACGTTCTTTAATAGTGGACTCTTGTTCCAAACTGGAACAACACTCAAC | |
| | CCTATCTCGGTCTATTCTTTTGATTTATAAGGGATTTTGCCGATTTCGGCC | |
| | TATTGGTTAAAAAATGAGCTGATTTAACAAAAATTTAACGCGAATTTTAAC | |
| | AAAATATTAACGCTTACAATTTAGGTGGCACTTTTCGGGGAAATGTGCGCG | |
| | GAACCCCTATTTGTTTATTTTTCTAAATACATTCAAATATGTATCCGCTCA | |
| | TGAGACAATAACCCTGATAAATGCTTCAATAATATTGAAAAAGGAAGAGTA | |
| | TGAGTATTCAACATTTCCGTGTCGCCCTTATTCCCTTTTTTGCGGCATTTT | |
| | GCCTTCCTGTTTTTGCTCACCCAGAAACGCTGGTGAAAGTAAAAGATGCTG | |
| | AAGATCAGTTGGGTGCACGAGTGGGTTACATCGAACTGGATCTCAACAGCG | |
| | GTAAGATCCTTGAGAGTTTTCGCCCCGAAGAACGTTTTCCAATGATGAGCA | |
| | CTTTTAAAGTTCTGCTATGTGGCGCGGTATTATCCCGTATTGACGCCGGGC | |
| | AAGAGCAACTCGGTCGCCGCATACACTATTCTCAGAATGACTTGGTTGAGT | |
| | ACTCACCAGTCACAGAAAAGCATCTTACGGATGGCATGACAGTAAGAGAAT | |
| | TATGCAGTGCTGCCATAACCATGAGTGATAACACTGCGGCCAACTTACTTC | |
| | TGACAACGATCGGAGGACCGAAGGAGCTAACCGCTTTTTTGCACAACATGG | |
| | GGGATCATGTAACTCGCCTTGATCGTTGGGAACCGGAGCTGAATGAAGCCA | |
| | TACCAAACGACGAGCGTGACACCACGATGCCTGTAGCAATGGCAACAACGT | |
| | TGCGCAAACTATTAACTGGCGAACTACTTACTCTAGCTTCCCGGCAACAAT | |
| | TAATAGACTGGATGGAGGCGGATAAAGTTGCAGGACCACTTCTGCGCTCGG | |
| | CCCTTCCGGCTGGCTGGTTTATTGCTGATAAATCTGGAGCCGGTGAGCGTG | |
| | GGTCTCGCGGTATCATTGCAGCACTGGGGCCAGATGGTAAGCCCTCCCGTA | |
| | TCGTAGTTATCTACACGACGGGGAGTCAGGCAACTATGGATGAACGAAATA | |
| | GACAGATCGCTGAGATAGGTGCCTCACTGATTAAGCATTGGTAACTGTCAG | |
| | ACCAAGTTTACTCATATATACTTTAGATTGATTTAAAACTTCATTTTTAAT | |
| | TTAAAAGGATCTAGGTGAAGATCCTTTTTGATAATCTCATGACCAAAATCC | |
| | CTTAACGTGAGTTTTCGTTCCACTGAGCGTCAGACCCCGTAGAAAAGATCA | |
| | AAGGATCTTCTTGAGATCCTTTTTTTCTGCGCGTAATCTGCTGCTTGCAAA | |
| | CAAAAAAACCACCGCTACCAGCGGTGGTTTGTTTGCCGGATCAAGAGCTAC | |
| | CAACTCTTTTTCCGAAGGTAACTGGCTTCAGCAGAGCGCAGATACCAAATA | |
| | CTGTTCTTCTAGTGTAGCCGTAGTTAGGCCACCACTTCAAGAACTCTGTAG | |
| | CACCGCCTACATACCTCGCTCTGCTAATCCTGTTACCAGTGGCTGCTGCCA | |
| | GTGGCGATAAGTCGTGTCTTACCGGGTTGGACTCAAGACGATAGTTACCGG | |
| | ATAAGGCGCAGCGGTCGGGCTGAACGGGGGGTTCGTGCACACAGCCCAGCT | |
| | TGGAGCGAACGACCTACACCGAACTGAGATACCTACAGCGTGAGCTATGAG | |
| | AAAGCGCCACGCTTCCCGAAGGGAGAAAGGCGGACAGGTATCCGGTAAGCG | |
| | GCAGGGTCGGAACAGGAGAGCGCACGAGGGAGCTTCCAGGGGGAAACGCCT | |
| | GGTATCTTTATAGTCCTGTCGGGTTTCGCCACCTCTGACTTGAGCGTCGAT | |
| | TTTTGTGATGCTCGTCAGGGGGGCGGAGCCTATGGAAAAACGCCAGCAACG | |
| | CGGCCTTTTTACGGTTCCTGGCCTTTTGCTGGCCTTTTGCTCACATGTTCT | |
| | TTCCTGCGTTATCCCCTGATTCTGTGGATAACCGTATTACCGCCTTTGAGT | |
| | GAGCTGATACCGCTCGCCGCAGCCGAACGACCGAGCGCAGCGAGTCAGTGA | |
| | GCGAGGAAGCGGAAGAGCGCCCAATACGCAAACCGCCTCTCCCCGCGCGTT | |
| | GGCCGATTCATTAATGCAGCTGGCACGACAGGTTTCCCGACTGGAAAGCGG | |
| | GCAGTGAGCGCAACGCAATTAATGTGAGTTAGCTCACTCATTAGGCACCCC | |
| | AGGCTTTACACTTTATGCTTCCGGCTCGTATGTTGTGTGGAATTGTGAGCG | |
| | GATAACAATTTCACACAGGAAACAGCTATGACCATGATTACGCCAGATTTA | |
| | ATTAAGGCCTTAATTAGGCTGCGCGCTCGCTCGCTCACTGAGGCCGCCCGG | |
| | GCAAAGCCCGGGCGTCGGGCGACCTTTGGTCGCCCGGCCTCAGTGAGCGAG | |
| | CGAGCGCGCAGAGAGGGAGTGGCCAACTCCATCACTAGGGGTTCCTTGTAG | |
| | TTAATGATTAACCCGCCATGCTACTTATCTACCAGGGTAATGGGGATCCTC | |
| | TAGAACTATAGCTAGTCGACATTGATTATTGACTAGTTATTAATAGTAATC | |
| | AATTACGGGGTCATTAGTTCATAGCCCATATATGGAGTTCCGCGTTACATA | |
| | ACTTACGGTAAATGGCCCGCCTGGCTGACCGCCCAACGACCCCCGCCCATT | |
| | GACGTCAATAATGACGTATGTTCCCATAGTAACGCCAATAGGGACTTTCCA | |
| | TTGACGTCAATGGGTGGAGTATTTACGGTAAACTGCCCACTTGGCAGTACA | |
| | TCAAGTGTATCATATGCCAAGTACGCCCCCTATTGACGTCAATGACGGTAA | |
| | ATGGCCCGCCTGGCATTATGCCCAGTACATGACCTTATGGGACTTTCCTAC | |
| | TTGGCAGTACATCTACGTATTAGTCATCGCTATTACCATGGTCGAGGTGAG | |
| | CCCCACGTTCTGCTTCACTCTCCCCATCTCCCCCCCCTCCCCACCCCCAAT | |
| | TTTGTATTTATTTATTTTTTAATTATTTTGTGCAGCGATGGGGGCGGGGGG | |
| | GGGGGGGGGGCGCGCGCCAGGCGGGGCGGGGCGGGGCGAGGGGCGGGGCGG | |
| | GGCGAGGCGGAGAGGTGCGGCGGCAGCCAATCAGAGCGGCGCGCTCCGAAA | |
| | GTTTCCTTTTATGGCGAGGCGGCGGCGGCGGCGGCCCTATAAAAAGCGAAG | |
| | CGCGCGGCGGGCGGGGAGTCGCTGCGACGCTGCCTTCGCCCCGTGCCCCGC | |
| | TCCGCCGCCGCCTCGCGCCGCCCGCCCCGGCTCTGACTGACCGCGTTACTC | |
| | CCACAGGTGAGCGGGCGGGACGGCCCTTCTCCTCCGGGCTGTAATTAGCGC | |
| | TTGGTTTAATGACGGCTTGTTTCTTTTCTGTGGCTGCGTGAAAGCCTTGAG | |
| | GGGCTCCGGGAGGGCCCTTTGTGCGGGGGGAGCGGCTCGGGGGGTGCGTGC | |
| | GTGTGTGTGTGCGTGGGGAGCGCCGCGTGCGGCTCCGCGCTGCCCGGCGGC | |
| | TGTGAGCGCTGCGGGCGCGGCGCGGGGCTTTGTGCGCTCCGCAGTGTGCGC | |
| | GAGGGGAGCGCGGCCGGGGGCGGTGCCCCGCGGTGCGGGGGGGGCTGCGAG | |
| | GGGAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGTT | |
| | GCTGAGCACGGCCCGGCTTCGGGTGCGGGGCTCCGTACGGGGCGTGGCGCG | |
| | GGGCTCGCCGTGCCGGGCGGGGGGTGGCGGCAGGTGGGGGTGCCGGGCGGG | |
| | GCGGGGCCGCCTCGGGCCGGGGAGGGCTCGGGGGAGGGGCGCGGCGGCCCC | |
| | CGGAGCGCCGGCGGCTGTCGAGGCGCGGCGAGCCGCAGCCATTGCCTTTTA | |
| | TGGTAATCGTGCGAGAGGGCGCAGGGACTTCCTTTGTCCCAAATCTGTGCG | |
| | GAGCCGAAATCTGGGAGGCGCCGCCGCACCCCCTCTAGCGGGCGCGGGGCG | |
| | AAGCGGTGCGGCGCCGGCAGGAAGGAAATGGGCGGGGAGGGCCTTCGTGCG | |
| | TCGCCGCGCCGCCGTCCCCTTCTCCCTCTCCAGCCTCGGGGCTGTCCGCGG | |
| | GGGGACGGCTGCCTTCGGGGGGGACGGGGCAGGGCGGGGTTCGGCTTCTGG | |
| | CGTGTGACCGGCGGCTCTAGAGCCTCTGCTAACCATGTTCATGCCTTCTTC | |
| | TTTTTCCTACAGCTCCTGGGCAACGTGCTGGTTATTGTGCTGTCTCATCAT | |
| | TTTGGCAAAGAATTCGCCACCATGGCCGTCGCAATCGCCGCCGCAAGAGTG | |
| | TGGAGACTGAATCGGGGACTGAGCCAGGCCGCACTGCTGCTGCTGAGACAG | |
| | CCAGGAGCCAGAGGCCTGGCCAGGAGCCACCCACCTAGGCAGCAGCAGCAG | |
| | TTCAGCTCCCTGGACGATAAGCCACAGTTTCCCGGCGCCTCTGCCGAGTTC | |
| | ATCGACAAGCTGGAGTTTATCCAGCCAAACGTGATCAGCGGCATCCCCATC | |
| | TACCGCGTGATGGACCGGCAGGGCCAGATCATCAATCCATCCGAGGACCCC | |
| | CACCTGCCAAAGGAGAAGGTGCTGAAGCTGTACAAGTCTATGACCCTGCTG | |
| | AACACAATGGATAGAATCCTGTATGAGTCCCAGCGCCAGGGCCGGATCTCT | |
| | TTCTACATGACCAACTATGGCGAGGAGGGCACACACGTGGGCAGCGCCGCC | |
| | GCCCTGGACAATACCGATCTGGTGTTCGGCCAGTATAGAGAGGCCGGCGTG | |
| | CTGATGTACAGGGACTATCCTCTGGAGCTGTTTATGGCCCAGTGCTACGGC | |
| | AATATCAGCGATCTGGGCAAGGGCCGCCAGATGCCAGTGCACTATGGCTGT | |
| | AAGGAGCGGCACTTCGTGACCATCTCTAGCCCCCTGGCCACACAGATCCCT | |
| | CAGGCAGTGGGAGCAGCCTACGCCGCCAAGAGAGCCAACGCCAATAGGGTG | |
| | GTCATCTGCTATTTTGGAGAGGGAGCAGCCTCCGAGGGCGACGCACACGCC | |
| | GGCTTCAACTTTGCCGCCACCCTGGAGTGCCCTATCATCTTCTTTTGTAGA | |
| | AACAATGGCTACGCCATCTCTACCCCAACAAGCGAGCAGTATAGGGGCGAT | |
| | GGAATCGCAGCCAGAGGCCCAGGCTACGGCATCATGTCCATCAGGGTGGAC | |
| | GGCAACGACGTGTTCGCCGTGTATAATGCCACAAAGGAGGCACGGAGAAGG | |
| | GCAGTGGCAGAGAACCAGCCCTTTCTGATCGAGGCCATGACCTACAGAATC | |
| | GGCCACCACAGCACATCCGACGATTCCTCTGCCTACAGGTCTGTGGACGAA | |
| | GTGAATTATTGGGACAAGCAGGATCACCCTATCAGCAGACTGAGGCACTAT | |
| | CTGCTGTCCCAGGGCTGGTGGGATGAGGAGCAGGAGAAGGCCTGGAGGAAG | |
| | CAGAGCCGCCGGAAAGTGATGGAGGCCTTCGAGCAGGCAGAGAGGAAGCCA | |
| | AAGCCCAACCCTAATCTGCTGTTTTCCGACGTGTACCAGGAGATGCCTGCC | |
| | CAGCTGAGGAAGCAGCAGGAGAGCCTGGCAAGACACCTGCAGACATACGGC | |
| | GAGCATTACCCCCTGGACCATTTTGATAAGTGAA | |
| 3 | CTGCGCGCTCGCTCGCTCACTGAGGCCGCCCGGGCAAAGCCCGGGCGTCGG | rAAV |
| | GCGACCTTTGGTCGCCCGGCCTCAGTGAGCGAGCGAGCGCGCAGAGAGGGA | vector: |
| | GTGTAGCCATGCTCTAGGAAGATCAATTCAATTCACGCGTCGACATTGATT | pJW2- |
| | ATTGACTAGTTATTAATAGTAATCAATTACGGGGTCATTAGTTCATAGCCC | pAAVsc. |
| | ATATATGGAGTTCCGCGTTACATAACTTACGGTAAATGGCCCGCCTGGCTG | CB6-opt- |
| | ACCGCCCAACGACCCCCGCCCATTGACGTCAATAATGACGTATGTTCCCAT | hBCKDHA-1 |
| | AGTAACGCCAATAGGGACTTTCCATTGACGTCAATGGGTGGATATTTACGG | |
| | TAAACTGCCCACTTGGCAGTACATCAAGTGTATCATATGCCAAGTACGCCC | |
| | CCTATTGACGTCAATGACGGTAAATGGCCCGCCTGGCATTATGCCCAGTAC | |
| | ATGACCTTATGGGACTTTCCTACTTGGCAGTACATCTACGTATTAGTCATC | |
| | GCTATTACCATGTCGAGGCCACGTTCTGCTTCACTCTCCCCATCTCCCCCC | |
| | CCTCCCCACCCCCAATTTTGTATTTATTTATTTTTTAATTATTTTGTGCAG | |
| | CGATGGGGGCGGGGGGGGGGGGCGCGCGCCAGGCGGGGCGGGGCGGGGCGA | |
| | GGGGCGGGGCGGGGCGAGGCGGAGAGGTGCGGCGGCAGCCAATCAGAGCGG | |
| | CGCGCTCCGAAAGTTTCCTTTTATGGCGAGGCGGCGGCGGCGGCGGCCCTA | |
| | TAAAAAGCGAAGCGCGCGGCGGGCGGGAGCAAGCTCTAGCCTCGAGGCCAC | |
| | CATGGCCGTCGCAATCGCCGCCGCAAGAGTGTGGAGACTGAATCGGGGACT | |
| | GAGCCAGGCCGCACTGCTGCTGCTGAGACAGCCAGGAGCCAGAGGCCTGGC | |
| | CAGGAGCCACCCACCTAGGCAGCAGCAGCAGTTCAGCTCCCTGGACGATAA | |
| | GCCACAGTTTCCCGGCGCCTCTGCCGAGTTCATCGACAAGCTGGAGTTTAT | |
| | CCAGCCAAACGTGATCAGCGGCATCCCCATCTACCGCGTGATGGACCGGCA | |
| | GGGCCAGATCATCAATCCATCCGAGGACCCCCACCTGCCAAAGGAGAAGGT | |
| | GCTGAAGCTGTACAAGTCTATGACCCTGCTGAACACAATGGATAGAATCCT | |
| | GTATGAGTCCCAGCGCCAGGGCCGGATCTCTTTCTACATGACCAACTATGG | |
| | CGAGGAGGGCACACACGTGGGCAGCGCCGCCGCCCTGGACAATACCGATCT | |
| | GGTGTTCGGCCAGTATAGAGAGGCCGGCGTGCTGATGTACAGGGACTATCC | |
| | TCTGGAGCTGTTTATGGCCCAGTGCTACGGCAATATCAGCGATCTGGGCAA | |
| | GGGCCGCCAGATGCCAGTGCACTATGGCTGTAAGGAGCGGCACTTCGTGAC | |
| | CATCTCTAGCCCCCTGGCCACACAGATCCCTCAGGCAGTGGGAGCAGCCTA | |
| | CGCCGCCAAGAGAGCCAACGCCAATAGGGTGGTCATCTGCTATTTTGGAGA | |
| | GGGAGCAGCCTCCGAGGGCGACGCACACGCCGGCTTCAACTTTGCCGCCAC | |
| | CCTGGAGTGCCCTATCATCTTCTTTTGTAGAAACAATGGCTACGCCATCTC | |
| | TACCCCAACAAGCGAGCAGTATAGGGGCGATGGAATCGCAGCCAGAGGCCC | |
| | AGGCTACGGCATCATGTCCATCAGGGTGGACGGCAACGACGTGTTCGCCGT | |
| | GTATAATGCCACAAAGGAGGCACGGAGAAGGGCAGTGGCAGAGAACCAGCC | |
| | CTTTCTGATCGAGGCCATGACCTACAGAATCGGCCACCACAGCACATCCGA | |
| | CGATTCCTCTGCCTACAGGTCTGTGGACGAAGTGAATTATTGGGACAAGCA | |
| | GGATCACCCTATCAGCAGACTGAGGCACTATCTGCTGTCCCAGGGCTGGTG | |
| | GGATGAGGAGCAGGAGAAGGCCTGGAGGAAGCAGAGCCGCCGGAAAGTGAT | |
| | GGAGGCCTTCGAGCAGGCAGAGAGGAAGCCAAAGCCCAACCCTAATCTGCT | |
| | GTTTTCCGACGTGTACCAGGAGATGCCTGCCCAGCTGAGGAAGCAGCAGGA | |
| | GAGCCTGGCAAGACACCTGCAGACATACGGCGAGCATTACCCCCTGGACCA | |
| | TTTTGATAAGTGAGGATCCGATCTTTTTCCCTCTGCCAAAAATTATGGGGA | |
| | CATCATGAAGCCCCTTGAGCATCTGACTTCTGGCTAATAAAGGAAATTTAT | |
| | TTTCATTGCAATAGTGTGTTGGAATTTTTTGTGTCTCTCACTCGGCCTAGG | |
| | TAGATAAGTAGCATGGCGGGTTAATCATTAACTACAAGGAACCCCTAGTGA | |
| | TGGAGTTGGCCACTCCCTCTCTGCGCGCTCGCTCGCTCACTGAGGCCGGGC | |
| | GACCAAAGGTCGCCCGACGCCCGGGCTTTGCCCGGGCGGCCTCAGTGAGCG | |
| | AGCGAGCGCGCAGCCTTAATTAACCTAATTCACTGGCCGTCGTTTTACAAC | |
| | GTCGTGACTGGGAAAACCCTGGCGTTACCCAACTTAATCGCCTTGCAGCAC | |
| | ATCCCCCTTTCGCCAGCTGGCGTAATAGCGAAGAGGCCCGCACCGATCGCC | |
| | CTTCCCAACAGTTGCGCAGCCTGAATGGCGAATGGGACGCGCCCTGTAGCG | |
| | GCGCATTAAGCGCGGCGGGTGTGGTGGTTACGCGCAGCGTGACCGCTACAC | |
| | TTGCCAGCGCCCTAGCGCCCGCTCCTTTCGCTTTCTTCCCTTCCTTTCTCG | |
| | CCACGTTCGCCGGCTTTCCCCGTCAAGCTCTAAATCGGGGGCTCCCTTTAG | |
| | GGTTCCGATTTAGTGCTTTACGGCACCTCGACCCCAAAAAACTTGATTAGG | |
| | GTGATGGTTCACGTAGTGGGCCATCGCCCTGATAGACGGTTTTTCGCCCTT | |
| | TGACGTTGGAGTCCACGTTCTTTAATAGTGGACTCTTGTTCCAAACTGGAA | |
| | CAACACTCAACCCTATCTCGGTCTATTCTTTTGATTTATAAGGGATTTTGC | |
| | CGATTTCGGCCTATTGGTTAAAAAATGAGCTGATTTAACAAAAATTTAACG | |
| | CGAATTTTAACAAAATATTAACGCTTACAATTTAGGTGGCACTTTTCGGGG | |
| | AAATGTGCGCGGAACCCCTATTTGTTTATTTTTCTAAATACATTCAAATAT | |
| | GTATCCGCTCATGAGACAATAACCCTGATAAATGCTTCAATAATATTGAAA | |
| | AAGGAAGAGTATGAGTATTCAACATTTCCGTGTCGCCCTTATTCCCTTTTT | |
| | TGCGGCATTTTGCCTTCCTGTTTTTGCTCACCCAGAAACGCTGGTGAAAGT | |
| | AAAAGATGCTGAAGATCAGTTGGGTGCACGAGTGGGTTACATCGAACTGGA | |
| | TCTCAACAGCGGTAAGATCCTTGAGAGTTTTCGCCCCGAAGAACGTTTTCC | |
| | AATGATGAGCACTTTTAAAGTTCTGCTATGTGGCGCGGTATTATCCCGTAT | |
| | TGACGCCGGGCAAGAGCAACTCGGTCGCCGCATACACTATTCTCAGAATGA | |
| | CTTGGTTGAGTACTCACCAGTCACAGAAAAGCATCTTACGGATGGCATGAC | |
| | AGTAAGAGAATTATGCAGTGCTGCCATAACCATGAGTGATAACACTGCGGC | |
| | CAACTTACTTCTGACAACGATCGGAGGACCGAAGGAGCTAACCGCTTTTTT | |
| | GCACAACATGGGGGATCATGTAACTCGCCTTGATCGTTGGGAACCGGAGCT | |
| | GAATGAAGCCATACCAAACGACGAGCGTGACACCACGATGCCTGTAGCAAT | |
| | GGCAACAACGTTGCGCAAACTATTAACTGGCGAACTACTTACTCTAGCTTC | |
| | CCGGCAACAATTAATAGACTGGATGGAGGCGGATAAAGTTGCAGGACCACT | |
| | TCTGCGCTCGGCCCTTCCGGCTGGCTGGTTTATTGCTGATAAATCTGGAGC | |
| | CGGTGAGCGTGGGTCTCGCGGTATCATTGCAGCACTGGGGCCAGATGGTAA | |
| | GCCCTCCCGTATCGTAGTTATCTACACGACGGGGAGTCAGGCAACTATGGA | |
| | TGAACGAAATAGACAGATCGCTGAGATAGGTGCCTCACTGATTAAGCATTG | |
| | GTAACTGTCAGACCAAGTTTACTCATATATACTTTAGATTGATTTAAAACT | |
| | TCATTTTTAATTTAAAAGGATCTAGGTGAAGATCCTTTTTGATAATCTCAT | |
| | GACCAAAATCCCTTAACGTGAGTTTTCGTTCCACTGAGCGTCAGACCCCGT | |
| | AGAAAAGATCAAAGGATCTTCTTGAGATCCTTTTTTTCTGCGCGTAATCTG | |
| | CTGCTTGCAAACAAAAAAACCACCGCTACCAGCGGTGGTTTGTTTGCCGGA | |
| | TCAAGAGCTACCAACTCTTTTTCCGAAGGTAACTGGCTTCAGCAGAGCGCA | |
| | GATACCAAATACTGTTCTTCTAGTGTAGCCGTAGTTAGGCCACCACTTCAA | |
| | GAACTCTGTAGCACCGCCTACATACCTCGCTCTGCTAATCCTGTTACCAGT | |
| | GGCTGCTGCCAGTGGCGATAAGTCGTGTCTTACCGGGTTGGACTCAAGACG | |
| | ATAGTTACCGGATAAGGCGCAGCGGTCGGGCTGAACGGGGGGTTCGTGCAC | |
| | ACAGCCCAGCTTGGAGCGAACGACCTACACCGAACTGAGATACCTACAGCG | |
| | TGAGCTATGAGAAAGCGCCACGCTTCCCGAAGGGAGAAAGGCGGACAGGTA | |
| | TCCGGTAAGCGGCAGGGTCGGAACAGGAGAGCGCACGAGGGAGCTTCCAGG | |
| | GGGAAACGCCTGGTATCTTTATAGTCCTGTCGGGTTTCGCCACCTCTGACT | |
| | TGAGCGTCGATTTTTGTGATGCTCGTCAGGGGGGCGGAGCCTATGGAAAAA | |
| | CGCCAGCAACGCGGCCTTTTTACGGTTCCTGGCCTTTTGCTGGCCTTTTGC | |
| | TCACATGTTCTTTCCTGCGTTATCCCCTGATTCTGTGGATAACCGTATTAC | |
| | CGCCTTTGAGTGAGCTGATACCGCTCGCCGCAGCCGAACGACCGAGCGCAG | |
| | CGAGTCAGTGAGCGAGGAAGCGGAAGAGCGCCCAATACGCAAACCGCCTCT | |
| | CCCCGCGCGTTGGCCGATTCATTAATGCAGCTGGCACGACAGGTTTCCCGA | |
| | CTGGAAAGCGGGCAGTGAGCGCAACGCAATTAATGTGAGTTAGCTCACTCA | |
| | TTAGGCACCCCAGGCTTTACACTTTATGCTTCCGGCTCGTATGTTGTGTGG | |
| | AATTGTGAGCGGATAACAATTTCACACAGGAAACAGCTATGACCATGATTA | |
| | CGCCAGATTTAATTAAGGCCTTAATTAGG | |
| 4 | atgGCCGTGGTCGCTGCTGCTGCCGGATGGCTGCTGAGACTGCGGGCCGCT | opti- |
| | GGGGCTGAGGGACATTGGAGGAGACTGCCTGGGGCTGGGCTGGCAAGGGGC | BCKDHB |
| | TTCCTGCACCCTGCAGCAACAGTGGAGGACGCAGCACAGCGGAGACAGGTG | |
| | GCCCACTTCACCTTTCAGCCCGATCCTGAGCCACGCGAGTACGGCCAGACA | |
| | CAGAAGATGAACCTGTTCCAGTCCGTGACCTCTGCCCTGGACAATAGCCTG | |
| | GCCAAGGATCCAACAGCCGTGATCTTTGGCGAGGACGTGGCCTTCGGCGGC | |
| | GTGTTTCGGTGCACAGTGGGCCTGAGAGACAAGTACGGCAAGGATCGGGTG | |
| | TTCAACACCCCACTGTGCGAGCAGGGAATCGTGGGCTTTGGCATCGGCATC | |
| | GCAGTGACCGGAGCAACAGCAATCGCAGAGATCCAGTTCGCCGACTATATC | |
| | TTCCCCGCCTTTGATCAGATCGTGAACGAGGCCGCCAAGTACAGGTATCGC | |
| | TCCGGCGACCTGTTTAATTGCGGCAGCCTGACCATCAGATCCCCTTGGGGA | |
| | TGCGTGGGACACGGCGCCCTGTATCACTCTCAGAGCCCAGAGGCCTTCTTT | |
| | GCCCACTGCCCCGGCATCAAGGTGGTCATCCCACGGAGCCCCTTCCAGGCA | |
| | AAGGGCCTGCTGCTGTCCTGCATCGAGGATAAGAACCCCTGTATCTTCTTT | |
| | GAGCCTAAGATCCTGTACAGAGCAGCAGCAGAGGAGGTGCCTATCGAGCCA | |
| | TATAATATCCCTCTGTCTCAGGCCGAAGTGATCCAGGAGGGAAGCGACGTG | |
| | ACCCTGGTGGCATGGGGAACACAGGTGCACGTGATCAGGGAGGTGGCCTCC | |
| | ATGGCCAAGGAGAAGCTGGGCGTGTCTTGCGAAGTGATCGATCTGAGGACC | |
| | ATCATCCCTTGGGACGTGGATACAATCTGTAAGTCTGTGATCAAGACCGGC | |
| | CGCCTGCTGATCAGCCACGAGGCACCACTGACAGGAGGATTCGCATCCGAG | |
| | ATCAGCTCCACCGTGCAGGAGGAGTGCTTTCTGAATCTGGAGGCCCCAATC | |
| | TCTCGGGTGTGCGGCTACGATACCCCCTTCCCTCACATCTTTGAGCCTTTC | |
| | TACATCCCTGACAAGTGGAAGTGCTACGACGCTCTGCGGAAGATGATTAAC | |
| | TATtga | |
| 5 | CGCGTGGTACCTCTAGAGTCGACCCGGGCGGCCTCGAGGACGGGGTGAACT | rAAV |
| | ACGCCTGAGGATCCGATCTTTTTCCCTCTGCCAAAAATTATGGGGACATCA | vector: |
| | TGAAGCCCCTTGAGCATCTGACTTCTGGCTAATAAAGGAAATTTATTTTCA | pJW152- |
| | TTGCAATAGTGTGTTGGAATTTTTTGTGTCTCTCACTCGGAAGCAATTCGT | pAAV.pCB- |
| | TGATCTGAATTTCGACCACCCATAATACCCATTACCCTGGTAGATAAGTAG | CBA-opt- |
| | CATGGCGGGTTAATCATTAACTACAAGGAACCCCTAGTGATGGAGTTGGCC | hBCKDHB |
| | ACTCCCTCTCTGCGCGCTCGCTCGCTCACTGAGGCCGGGCGACCAAAGGTC | |
| | GCCCGACGCCCGGGCTTTGCCCGGGCGGCCTCAGTGAGCGAGCGAGCGCGC | |
| | AGCCTTAATTAACCTAATTCACTGGCCGTCGTTTTACAACGTCGTGACTGG | |
| | GAAAACCCTGGCGTTACCCAACTTAATCGCCTTGCAGCACATCCCCCTTTC | |
| | GCCAGCTGGCGTAATAGCGAAGAGGCCCGCACCGATCGCCCTTCCCAACAG | |
| | TTGCGCAGCCTGAATGGCGAATGGGACGCGCCCTGTAGCGGCGCATTAAGC | |
| | GCGGCGGGTGTGGTGGTTACGCGCAGCGTGACCGCTACACTTGCCAGCGCC | |
| | CTAGCGCCCGCTCCTTTCGCTTTCTTCCCTTCCTTTCTCGCCACGTTCGCC | |
| | GGCTTTCCCCGTCAAGCTCTAAATCGGGGGCTCCCTTTAGGGTTCCGATTT | |
| | AGTGCTTTACGGCACCTCGACCCCAAAAAACTTGATTAGGGTGATGGTTCA | |
| | CGTAGTGGGCCATCGCCCTGATAGACGGTTTTTCGCCCTTTGACGTTGGAG | |
| | TCCACGTTCTTTAATAGTGGACTCTTGTTCCAAACTGGAACAACACTCAAC | |
| | CCTATCTCGGTCTATTCTTTTGATTTATAAGGGATTTTGCCGATTTCGGCC | |
| | TATTGGTTAAAAAATGAGCTGATTTAACAAAAATTTAACGCGAATTTTAAC | |
| | AAAATATTAACGCTTACAATTTAGGTGGCACTTTTCGGGGAAATGTGCGCG | |
| | GAACCCCTATTTGTTTATTTTTCTAAATACATTCAAATATGTATCCGCTCA | |
| | TGAGACAATAACCCTGATAAATGCTTCAATAATATTGAAAAAGGAAGAGTA | |
| | TGAGTATTCAACATTTCCGTGTCGCCCTTATTCCCTTTTTTGCGGCATTTT | |
| | GCCTTCCTGTTTTTGCTCACCCAGAAACGCTGGTGAAAGTAAAAGATGCTG | |
| | AAGATCAGTTGGGTGCACGAGTGGGTTACATCGAACTGGATCTCAACAGCG | |
| | GTAAGATCCTTGAGAGTTTTCGCCCCGAAGAACGTTTTCCAATGATGAGCA | |
| | CTTTTAAAGTTCTGCTATGTGGCGCGGTATTATCCCGTATTGACGCCGGGC | |
| | AAGAGCAACTCGGTCGCCGCATACACTATTCTCAGAATGACTTGGTTGAGT | |
| | ACTCACCAGTCACAGAAAAGCATCTTACGGATGGCATGACAGTAAGAGAAT | |
| | TATGCAGTGCTGCCATAACCATGAGTGATAACACTGCGGCCAACTTACTTC | |
| | TGACAACGATCGGAGGACCGAAGGAGCTAACCGCTTTTTTGCACAACATGG | |
| | GGGATCATGTAACTCGCCTTGATCGTTGGGAACCGGAGCTGAATGAAGCCA | |
| | TACCAAACGACGAGCGTGACACCACGATGCCTGTAGCAATGGCAACAACGT | |
| | TGCGCAAACTATTAACTGGCGAACTACTTACTCTAGCTTCCCGGCAACAAT | |
| | TAATAGACTGGATGGAGGCGGATAAAGTTGCAGGACCACTTCTGCGCTCGG | |
| | CCCTTCCGGCTGGCTGGTTTATTGCTGATAAATCTGGAGCCGGTGAGCGTG | |
| | GGTCTCGCGGTATCATTGCAGCACTGGGGCCAGATGGTAAGCCCTCCCGTA | |
| | TCGTAGTTATCTACACGACGGGGAGTCAGGCAACTATGGATGAACGAAATA | |
| | GACAGATCGCTGAGATAGGTGCCTCACTGATTAAGCATTGGTAACTGTCAG | |
| | ACCAAGTTTACTCATATATACTTTAGATTGATTTAAAACTTCATTTTTAAT | |
| | TTAAAAGGATCTAGGTGAAGATCCTTTTTGATAATCTCATGACCAAAATCC | |
| | CTTAACGTGAGTTTTCGTTCCACTGAGCGTCAGACCCCGTAGAAAAGATCA | |
| | AAGGATCTTCTTGAGATCCTTTTTTTCTGCGCGTAATCTGCTGCTTGCAAA | |
| | CAAAAAAACCACCGCTACCAGCGGTGGTTTGTTTGCCGGATCAAGAGCTAC | |
| | CAACTCTTTTTCCGAAGGTAACTGGCTTCAGCAGAGCGCAGATACCAAATA | |
| | CTGTTCTTCTAGTGTAGCCGTAGTTAGGCCACCACTTCAAGAACTCTGTAG | |
| | CACCGCCTACATACCTCGCTCTGCTAATCCTGTTACCAGTGGCTGCTGCCA | |
| | GTGGCGATAAGTCGTGTCTTACCGGGTTGGACTCAAGACGATAGTTACCGG | |
| | ATAAGGCGCAGCGGTCGGGCTGAACGGGGGGTTCGTGCACACAGCCCAGCT | |
| | TGGAGCGAACGACCTACACCGAACTGAGATACCTACAGCGTGAGCTATGAG | |
| | AAAGCGCCACGCTTCCCGAAGGGAGAAAGGCGGACAGGTATCCGGTAAGCG | |
| | GCAGGGTCGGAACAGGAGAGCGCACGAGGGAGCTTCCAGGGGGAAACGCCT | |
| | GGTATCTTTATAGTCCTGTCGGGTTTCGCCACCTCTGACTTGAGCGTCGAT | |
| | TTTTGTGATGCTCGTCAGGGGGGCGGAGCCTATGGAAAAACGCCAGCAACG | |
| | CGGCCTTTTTACGGTTCCTGGCCTTTTGCTGGCCTTTTGCTCACATGTTCT | |
| | TTCCTGCGTTATCCCCTGATTCTGTGGATAACCGTATTACCGCCTTTGAGT | |
| | GAGCTGATACCGCTCGCCGCAGCCGAACGACCGAGCGCAGCGAGTCAGTGA | |
| | GCGAGGAAGCGGAAGAGCGCCCAATACGCAAACCGCCTCTCCCCGCGCGTT | |
| | GGCCGATTCATTAATGCAGCTGGCACGACAGGTTTCCCGACTGGAAAGCGG | |
| | GCAGTGAGCGCAACGCAATTAATGTGAGTTAGCTCACTCATTAGGCACCCC | |
| | AGGCTTTACACTTTATGCTTCCGGCTCGTATGTTGTGTGGAATTGTGAGCG | |
| | GATAACAATTTCACACAGGAAACAGCTATGACCATGATTACGCCAGATTTA | |
| | ATTAAGGCCTTAATTAGGCTGCGCGCTCGCTCGCTCACTGAGGCCGCCCGG | |
| | GCAAAGCCCGGGCGTCGGGCGACCTTTGGTCGCCCGGCCTCAGTGAGCGAG | |
| | CGAGCGCGCAGAGAGGGAGTGGCCAACTCCATCACTAGGGGTTCCTTGTAG | |
| | TTAATGATTAACCCGCCATGCTACTTATCTACCAGGGTAATGGGGATCCTC | |
| | TAGAACTATAGCTAGTCGACATTGATTATTGACTAGTTATTAATAGTAATC | |
| | AATTACGGGGTCATTAGTTCATAGCCCATATATGGAGTTCCGCGTTACATA | |
| | ACTTACGGTAAATGGCCCGCCTGGCTGACCGCCCAACGACCCCCGCCCATT | |
| | GACGTCAATAATGACGTATGTTCCCATAGTAACGCCAATAGGGACTTTCCA | |
| | TTGACGTCAATGGGTGGAGTATTTACGGTAAACTGCCCACTTGGCAGTACA | |
| | TCAAGTGTATCATATGCCAAGTACGCCCCCTATTGACGTCAATGACGGTAA | |
| | ATGGCCCGCCTGGCATTATGCCCAGTACATGACCTTATGGGACTTTCCTAC | |
| | TTGGCAGTACATCTACGTATTAGTCATCGCTATTACCATGGTCGAGGTGAG | |
| | CCCCACGTTCTGCTTCACTCTCCCCATCTCCCCCCCCTCCCCACCCCCAAT | |
| | TTTGTATTTATTTATTTTTTAATTATTTTGTGCAGCGATGGGGGCGGGGGG | |
| | GGGGGGGGGGCGCGCGCCAGGCGGGGCGGGGCGGGGCGAGGGGCGGGGCGG | |
| | GGCGAGGCGGAGAGGTGCGGCGGCAGCCAATCAGAGCGGCGCGCTCCGAAA | |
| | GTTTCCTTTTATGGCGAGGCGGCGGCGGCGGCGGCCCTATAAAAAGCGAAG | |
| | CGCGCGGCGGGCGGGGAGTCGCTGCGACGCTGCCTTCGCCCCGTGCCCCGC | |
| | TCCGCCGCCGCCTCGCGCCGCCCGCCCCGGCTCTGACTGACCGCGTTACTC | |
| | CCACAGGTGAGCGGGCGGGACGGCCCTTCTCCTCCGGGCTGTAATTAGCGC | |
| | TTGGTTTAATGACGGCTTGTTTCTTTTCTGTGGCTGCGTGAAAGCCTTGAG | |
| | GGGCTCCGGGAGGGCCCTTTGTGCGGGGGGAGCGGCTCGGGGGGTGCGTGC | |
| | GTGTGTGTGTGCGTGGGGAGCGCCGCGTGCGGCTCCGCGCTGCCCGGCGGC | |
| | TGTGAGCGCTGCGGGCGCGGCGCGGGGCTTTGTGCGCTCCGCAGTGTGCGC | |
| | GAGGGGAGCGCGGCCGGGGGCGGTGCCCCGCGGTGCGGGGGGGGCTGCGAG | |
| | GGGAACAAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCCCGAGTT | |
| | GCTGAGCACGGCCCGGCTTCGGGTGCGGGGCTCCGTACGGGGCGTGGCGCG | |
| | GGGCTCGCCGTGCCGGGCGGGGGGTGGCGGCAGGTGGGGGTGCCGGGCGGG | |
| | GCGGGGCCGCCTCGGGCCGGGGAGGGCTCGGGGGAGGGGCGCGGCGGCCCC | |
| | CGGAGCGCCGGCGGCTGTCGAGGCGCGGCGAGCCGCAGCCATTGCCTTTTA | |
| | TGGTAATCGTGCGAGAGGGCGCAGGGACTTCCTTTGTCCCAAATCTGTGCG | |
| | GAGCCGAAATCTGGGAGGCGCCGCCGCACCCCCTCTAGCGGGCGCGGGGCG | |
| | AAGCGGTGCGGCGCCGGCAGGAAGGAAATGGGCGGGGAGGGCCTTCGTGCG | |
| | TCGCCGCGCCGCCGTCCCCTTCTCCCTCTCCAGCCTCGGGGCTGTCCGCGG | |
| | GGGGACGGCTGCCTTCGGGGGGGACGGGGCAGGGCGGGGTTCGGCTTCTGG | |
| | CGTGTGACCGGCGGCTCTAGAGCCTCTGCTAACCATGTTCATGCCTTCTTC | |
| | TTTTTCCTACAGCTCCTGGGCAACGTGCTGGTTATTGTGCTGTCTCATCAT | |
| | TTTGGCAAAGAATTCGCCACCATGGCCGTGGTCGCTGCTGCTGCCGGATGG | |
| | CTGCTGAGACTGCGGGCCGCTGGGGCTGAGGGACATTGGAGGAGACTGCCT | |
| | GGGGCTGGGCTGGCAAGGGGCTTCCTGCACCCTGCAGCAACAGTGGAGGAC | |
| | GCAGCACAGCGGAGACAGGTGGCCCACTTCACCTTTCAGCCCGATCCTGAG | |
| | CCACGCGAGTACGGCCAGACACAGAAGATGAACCTGTTCCAGTCCGTGACC | |
| | TCTGCCCTGGACAATAGCCTGGCCAAGGATCCAACAGCCGTGATCTTTGGC | |
| | GAGGACGTGGCCTTCGGCGGCGTGTTTCGGTGCACAGTGGGCCTGAGAGAC | |
| | AAGTACGGCAAGGATCGGGTGTTCAACACCCCACTGTGCGAGCAGGGAATC | |
| | GTGGGCTTTGGCATCGGCATCGCAGTGACCGGAGCAACAGCAATCGCAGAG | |
| | ATCCAGTTCGCCGACTATATCTTCCCCGCCTTTGATCAGATCGTGAACGAG | |
| | GCCGCCAAGTACAGGTATCGCTCCGGCGACCTGTTTAATTGCGGCAGCCTG | |
| | ACCATCAGATCCCCTTGGGGATGCGTGGGACACGGCGCCCTGTATCACTCT | |
| | CAGAGCCCAGAGGCCTTCTTTGCCCACTGCCCCGGCATCAAGGTGGTCATC | |
| | CCACGGAGCCCCTTCCAGGCAAAGGGCCTGCTGCTGTCCTGCATCGAGGAT | |
| | AAGAACCCCTGTATCTTCTTTGAGCCTAAGATCCTGTACAGAGCAGCAGCA | |
| | GAGGAGGTGCCTATCGAGCCATATAATATCCCTCTGTCTCAGGCCGAAGTG | |
| | ATCCAGGAGGGAAGCGACGTGACCCTGGTGGCATGGGGAACACAGGTGCAC | |
| | GTGATCAGGGAGGTGGCCTCCATGGCCAAGGAGAAGCTGGGCGTGTCTTGC | |
| | GAAGTGATCGATCTGAGGACCATCATCCCTTGGGACGTGGATACAATCTGT | |
| | AAGTCTGTGATCAAGACCGGCCGCCTGCTGATCAGCCACGAGGCACCACTG | |
| | ACAGGAGGATTCGCATCCGAGATCAGCTCCACCGTGCAGGAGGAGTGCTTT | |
| | CTGAATCTGGAGGCCCCAATCTCTCGGGTGTGCGGCTACGATACCCCCTTC | |
| | CCTCACATCTTTGAGCCTTTCTACATCCCTGACAAGTGGAAGTGCTACGAC | |
| | GCTCTGCGGAAGATGATTAACTATTGAA | |
| 6 | CTGCGCGCTCGCTCGCTCACTGAGGCCGCCCGGGCAAAGCCCGGGCGTCGG | rAAV |
| | GCGACCTTTGGTCGCCCGGCCTCAGTGAGCGAGCGAGCGCGCAGAGAGGGA | vector: |
| | GTGTAGCCATGCTCTAGGAAGATCAATTCAATTCACGCGTCGACATTGATT | pJW153- |
| | ATTGACTAGTTATTAATAGTAATCAATTACGGGGTCATTAGTTCATAGCCC | pAAVsc. |
| | ATATATGGAGTTCCGCGTTACATAACTTACGGTAAATGGCCCGCCTGGCTG | CB6-opt- |
| | ACCGCCCAACGACCCCCGCCCATTGACGTCAATAATGACGTATGTTCCCAT | hBCKDHB |
| | AGTAACGCCAATAGGGACTTTCCATTGACGTCAATGGGTGGATATTTACGG | |
| | TAAACTGCCCACTTGGCAGTACATCAAGTGTATCATATGCCAAGTACGCCC | |
| | CCTATTGACGTCAATGACGGTAAATGGCCCGCCTGGCATTATGCCCAGTAC | |
| | ATGACCTTATGGGACTTTCCTACTTGGCAGTACATCTACGTATTAGTCATC | |
| | GCTATTACCATGTCGAGGCCACGTTCTGCTTCACTCTCCCCATCTCCCCCC | |
| | CCTCCCCACCCCCAATTTTGTATTTATTTATTTTTTAATTATTTTGTGCAG | |
| | CGATGGGGGCGGGGGGGGGGGGCGCGCGCCAGGCGGGGCGGGGCGGGGCGA | |
| | GGGGCGGGGCGGGGCGAGGCGGAGAGGTGCGGCGGCAGCCAATCAGAGCGG | |
| | CGCGCTCCGAAAGTTTCCTTTTATGGCGAGGCGGCGGCGGCGGCGGCCCTA | |
| | TAAAAAGCGAAGCGCGCGGCGGGCGGGAGCAAGCTCTAGCCTCGAGGCCAC | |
| | CATGGCCGTGGTCGCTGCTGCTGCCGGATGGCTGCTGAGACTGCGGGCCGC | |
| | TGGGGCTGAGGGACATTGGAGGAGACTGCCTGGGGCTGGGCTGGCAAGGGG | |
| | CTTCCTGCACCCTGCAGCAACAGTGGAGGACGCAGCACAGCGGAGACAGGT | |
| | GGCCCACTTCACCTTTCAGCCCGATCCTGAGCCACGCGAGTACGGCCAGAC | |
| | ACAGAAGATGAACCTGTTCCAGTCCGTGACCTCTGCCCTGGACAATAGCCT | |
| | GGCCAAGGATCCAACAGCCGTGATCTTTGGCGAGGACGTGGCCTTCGGCGG | |
| | CGTGTTTCGGTGCACAGTGGGCCTGAGAGACAAGTACGGCAAGGATCGGGT | |
| | GTTCAACACCCCACTGTGCGAGCAGGGAATCGTGGGCTTTGGCATCGGCAT | |
| | CGCAGTGACCGGAGCAACAGCAATCGCAGAGATCCAGTTCGCCGACTATAT | |
| | CTTCCCCGCCTTTGATCAGATCGTGAACGAGGCCGCCAAGTACAGGTATCG | |
| | CTCCGGCGACCTGTTTAATTGCGGCAGCCTGACCATCAGATCCCCTTGGGG | |
| | ATGCGTGGGACACGGCGCCCTGTATCACTCTCAGAGCCCAGAGGCCTTCTT | |
| | TGCCCACTGCCCCGGCATCAAGGTGGTCATCCCACGGAGCCCCTTCCAGGC | |
| | AAAGGGCCTGCTGCTGTCCTGCATCGAGGATAAGAACCCCTGTATCTTCTT | |
| | TGAGCCTAAGATCCTGTACAGAGCAGCAGCAGAGGAGGTGCCTATCGAGCC | |
| | ATATAATATCCCTCTGTCTCAGGCCGAAGTGATCCAGGAGGGAAGCGACGT | |
| | GACCCTGGTGGCATGGGGAACACAGGTGCACGTGATCAGGGAGGTGGCCTC | |
| | CATGGCCAAGGAGAAGCTGGGCGTGTCTTGCGAAGTGATCGATCTGAGGAC | |
| | CATCATCCCTTGGGACGTGGATACAATCTGTAAGTCTGTGATCAAGACCGG | |
| | CCGCCTGCTGATCAGCCACGAGGCACCACTGACAGGAGGATTCGCATCCGA | |
| | GATCAGCTCCACCGTGCAGGAGGAGTGCTTTCTGAATCTGGAGGCCCCAAT | |
| | CTCTCGGGTGTGCGGCTACGATACCCCCTTCCCTCACATCTTTGAGCCTTT | |
| | CTACATCCCTGACAAGTGGAAGTGCTACGACGCTCTGCGGAAGATGATTAA | |
| | CTATTGAGGATCCGATCTTTTTCCCTCTGCCAAAAATTATGGGGACATCAT | |
| | GAAGCCCCTTGAGCATCTGACTTCTGGCTAATAAAGGAAATTTATTTTCAT | |
| | TGCAATAGTGTGTTGGAATTTTTTGTGTCTCTCACTCGGCCTAGGTAGATA | |
| | AGTAGCATGGCGGGTTAATCATTAACTACAAGGAACCCCTAGTGATGGAGT | |
| | TGGCCACTCCCTCTCTGCGCGCTCGCTCGCTCACTGAGGCCGGGCGACCAA | |
| | AGGTCGCCCGACGCCCGGGCTTTGCCCGGGCGGCCTCAGTGAGCGAGCGAG | |
| | CGCGCAGCCTTAATTAACCTAATTCACTGGCCGTCGTTTTACAACGTCGTG | |
| | ACTGGGAAAACCCTGGCGTTACCCAACTTAATCGCCTTGCAGCACATCCCC | |
| | CTTTCGCCAGCTGGCGTAATAGCGAAGAGGCCCGCACCGATCGCCCTTCCC | |
| | AACAGTTGCGCAGCCTGAATGGCGAATGGGACGCGCCCTGTAGCGGCGCAT | |
| | TAAGCGCGGCGGGTGTGGTGGTTACGCGCAGCGTGACCGCTACACTTGCCA | |
| | GCGCCCTAGCGCCCGCTCCTTTCGCTTTCTTCCCTTCCTTTCTCGCCACGT | |
| | TCGCCGGCTTTCCCCGTCAAGCTCTAAATCGGGGGCTCCCTTTAGGGTTCC | |
| | GATTTAGTGCTTTACGGCACCTCGACCCCAAAAAACTTGATTAGGGTGATG | |
| | GTTCACGTAGTGGGCCATCGCCCTGATAGACGGTTTTTCGCCCTTTGACGT | |
| | TGGAGTCCACGTTCTTTAATAGTGGACTCTTGTTCCAAACTGGAACAACAC | |
| | TCAACCCTATCTCGGTCTATTCTTTTGATTTATAAGGGATTTTGCCGATTT | |
| | CGGCCTATTGGTTAAAAAATGAGCTGATTTAACAAAAATTTAACGCGAATT | |
| | TTAACAAAATATTAACGCTTACAATTTAGGTGGCACTTTTCGGGGAAATGT | |
| | GCGCGGAACCCCTATTTGTTTATTTTTCTAAATACATTCAAATATGTATCC | |
| | GCTCATGAGACAATAACCCTGATAAATGCTTCAATAATATTGAAAAAGGAA | |
| | GAGTATGAGTATTCAACATTTCCGTGTCGCCCTTATTCCCTTTTTTGCGGC | |
| | ATTTTGCCTTCCTGTTTTTGCTCACCCAGAAACGCTGGTGAAAGTAAAAGA | |
| | TGCTGAAGATCAGTTGGGTGCACGAGTGGGTTACATCGAACTGGATCTCAA | |
| | CAGCGGTAAGATCCTTGAGAGTTTTCGCCCCGAAGAACGTTTTCCAATGAT | |
| | GAGCACTTTTAAAGTTCTGCTATGTGGCGCGGTATTATCCCGTATTGACGC | |
| | CGGGCAAGAGCAACTCGGTCGCCGCATACACTATTCTCAGAATGACTTGGT | |
| | TGAGTACTCACCAGTCACAGAAAAGCATCTTACGGATGGCATGACAGTAAG | |
| | AGAATTATGCAGTGCTGCCATAACCATGAGTGATAACACTGCGGCCAACTT | |
| | ACTTCTGACAACGATCGGAGGACCGAAGGAGCTAACCGCTTTTTTGCACAA | |
| | CATGGGGGATCATGTAACTCGCCTTGATCGTTGGGAACCGGAGCTGAATGA | |
| | AGCCATACCAAACGACGAGCGTGACACCACGATGCCTGTAGCAATGGCAAC | |
| | AACGTTGCGCAAACTATTAACTGGCGAACTACTTACTCTAGCTTCCCGGCA | |
| | ACAATTAATAGACTGGATGGAGGCGGATAAAGTTGCAGGACCACTTCTGCG | |
| | CTCGGCCCTTCCGGCTGGCTGGTTTATTGCTGATAAATCTGGAGCCGGTGA | |
| | GCGTGGGTCTCGCGGTATCATTGCAGCACTGGGGCCAGATGGTAAGCCCTC | |
| | CCGTATCGTAGTTATCTACACGACGGGGAGTCAGGCAACTATGGATGAACG | |
| | AAATAGACAGATCGCTGAGATAGGTGCCTCACTGATTAAGCATTGGTAACT | |
| | GTCAGACCAAGTTTACTCATATATACTTTAGATTGATTTAAAACTTCATTT | |
| | TTAATTTAAAAGGATCTAGGTGAAGATCCTTTTTGATAATCTCATGACCAA | |
| | AATCCCTTAACGTGAGTTTTCGTTCCACTGAGCGTCAGACCCCGTAGAAAA | |
| | GATCAAAGGATCTTCTTGAGATCCTTTTTTTCTGCGCGTAATCTGCTGCTT | |
| | GCAAACAAAAAAACCACCGCTACCAGCGGTGGTTTGTTTGCCGGATCAAGA | |
| | GCTACCAACTCTTTTTCCGAAGGTAACTGGCTTCAGCAGAGCGCAGATACC | |
| | AAATACTGTTCTTCTAGTGTAGCCGTAGTTAGGCCACCACTTCAAGAACTC | |
| | TGTAGCACCGCCTACATACCTCGCTCTGCTAATCCTGTTACCAGTGGCTGC | |
| | TGCCAGTGGCGATAAGTCGTGTCTTACCGGGTTGGACTCAAGACGATAGTT | |
| | ACCGGATAAGGCGCAGCGGTCGGGCTGAACGGGGGGTTCGTGCACACAGCC | |
| | CAGCTTGGAGCGAACGACCTACACCGAACTGAGATACCTACAGCGTGAGCT | |
| | ATGAGAAAGCGCCACGCTTCCCGAAGGGAGAAAGGCGGACAGGTATCCGGT | |
| | AAGCGGCAGGGTCGGAACAGGAGAGCGCACGAGGGAGCTTCCAGGGGGAAA | |
| | CGCCTGGTATCTTTATAGTCCTGTCGGGTTTCGCCACCTCTGACTTGAGCG | |
| | TCGATTTTTGTGATGCTCGTCAGGGGGGCGGAGCCTATGGAAAAACGCCAG | |
| | CAACGCGGCCTTTTTACGGTTCCTGGCCTTTTGCTGGCCTTTTGCTCACAT | |
| | GTTCTTTCCTGCGTTATCCCCTGATTCTGTGGATAACCGTATTACCGCCTT | |
| | TGAGTGAGCTGATACCGCTCGCCGCAGCCGAACGACCGAGCGCAGCGAGTC | |
| | AGTGAGCGAGGAAGCGGAAGAGCGCCCAATACGCAAACCGCCTCTCCCCGC | |
| | GCGTTGGCCGATTCATTAATGCAGCTGGCACGACAGGTTTCCCGACTGGAA | |
| | AGCGGGCAGTGAGCGCAACGCAATTAATGTGAGTTAGCTCACTCATTAGGC | |
| | ACCCCAGGCTTTACACTTTATGCTTCCGGCTCGTATGTTGTGTGGAATTGT | |
| | GAGCGGATAACAATTTCACACAGGAAACAGCTATGACCATGATTACGCCAG | |
| | ATTTAATTAAGGCCTTAATTAGG | |
| 7 | CGCGTGGTACCTCTAGAGTCGACCCGGGCGGCCTCGAGGACGGGGTGAACT | rAAV |
| | ACGCCTGAGGATCCGATCTTTTTCCCTCTGCCAAAAATTATGGGGACATCA | vector: |
| | TGAAGCCCCTTGAGCATCTGACTTCTGGCTAATAAAGGAAATTTATTTTCA | pJW154- |
| | TTGCAATAGTGTGTTGGAATTTTTTGTGTCTCTCACTCGGAAGCAATTCGT | pAAV- |
| | TGATCTGAATTTCGACCACCCATAATACCCATTACCCTGGTAGATAAGTAG | pCB-opt- |
| | CATGGCGGGTTAATCATTAACTACAAGGAACCCCTAGTGATGGAGTTGGCC | BCKDHB- |
| | ACTCCCTCTCTGCGCGCTCGCTCGCTCACTGAGGCCGGGCGACCAAAGGTC | T2A-opt- |
| | GCCCGACGCCCGGGCTTTGCCCGGGCGGCCTCAGTGAGCGAGCGAGCGCGC | BCKDHA- |
| | AGCCTTAATTAACCTAATTCACTGGCCGTCGTTTTACAACGTCGTGACTGG | 1 |
| | GAAAACCCTGGCGTTACCCAACTTAATCGCCTTGCAGCACATCCCCCTTTC | |
| | GCCAGCTGGCGTAATAGCGAAGAGGCCCGCACCGATCGCCCTTCCCAACAG | |
| | TTGCGCAGCCTGAATGGCGAATGGGACGCGCCCTGTAGCGGCGCATTAAGC | |
| | GCGGCGGGTGTGGTGGTTACGCGCAGCGTGACCGCTACACTTGCCAGCGCC | |
| | CTAGCGCCCGCTCCTTTCGCTTTCTTCCCTTCCTTTCTCGCCACGTTCGCC | |
| | GGCTTTCCCCGTCAAGCTCTAAATCGGGGGCTCCCTTTAGGGTTCCGATTT | |
| | AGTGCTTTACGGCACCTCGACCCCAAAAAACTTGATTAGGGTGATGGTTCA | |
| | CGTAGTGGGCCATCGCCCTGATAGACGGTTTTTCGCCCTTTGACGTTGGAG | |
| | TCCACGTTCTTTAATAGTGGACTCTTGTTCCAAACTGGAACAACACTCAAC | |
| | CCTATCTCGGTCTATTCTTTTGATTTATAAGGGATTTTGCCGATTTCGGCC | |
| | TATTGGTTAAAAAATGAGCTGATTTAACAAAAATTTAACGCGAATTTTAAC | |
| | AAAATATTAACGCTTACAATTTAGGTGGCACTTTTCGGGGAAATGTGCGCG | |
| | GAACCCCTATTTGTTTATTTTTCTAAATACATTCAAATATGTATCCGCTCA | |
| | TGAGACAATAACCCTGATAAATGCTTCAATAATATTGAAAAAGGAAGAGTA | |
| | TGAGTATTCAACATTTCCGTGTCGCCCTTATTCCCTTTTTTGCGGCATTTT | |
| | GCCTTCCTGTTTTTGCTCACCCAGAAACGCTGGTGAAAGTAAAAGATGCTG | |
| | AAGATCAGTTGGGTGCACGAGTGGGTTACATCGAACTGGATCTCAACAGCG | |
| | GTAAGATCCTTGAGAGTTTTCGCCCCGAAGAACGTTTTCCAATGATGAGCA | |
| | CTTTTAAAGTTCTGCTATGTGGCGCGGTATTATCCCGTATTGACGCCGGGC | |
| | AAGAGCAACTCGGTCGCCGCATACACTATTCTCAGAATGACTTGGTTGAGT | |
| | ACTCACCAGTCACAGAAAAGCATCTTACGGATGGCATGACAGTAAGAGAAT | |
| | TATGCAGTGCTGCCATAACCATGAGTGATAACACTGCGGCCAACTTACTTC | |
| | TGACAACGATCGGAGGACCGAAGGAGCTAACCGCTTTTTTGCACAACATGG | |
| | GGGATCATGTAACTCGCCTTGATCGTTGGGAACCGGAGCTGAATGAAGCCA | |
| | TACCAAACGACGAGCGTGACACCACGATGCCTGTAGCAATGGCAACAACGT | |
| | TGCGCAAACTATTAACTGGCGAACTACTTACTCTAGCTTCCCGGCAACAAT | |
| | TAATAGACTGGATGGAGGCGGATAAAGTTGCAGGACCACTTCTGCGCTCGG | |
| | CCCTTCCGGCTGGCTGGTTTATTGCTGATAAATCTGGAGCCGGTGAGCGTG | |
| | GGTCTCGCGGTATCATTGCAGCACTGGGGCCAGATGGTAAGCCCTCCCGTA | |
| | TCGTAGTTATCTACACGACGGGGAGTCAGGCAACTATGGATGAACGAAATA | |
| | GACAGATCGCTGAGATAGGTGCCTCACTGATTAAGCATTGGTAACTGTCAG | |
| | ACCAAGTTTACTCATATATACTTTAGATTGATTTAAAACTTCATTTTTAAT | |
| | TTAAAAGGATCTAGGTGAAGATCCTTTTTGATAATCTCATGACCAAAATCC | |
| | CTTAACGTGAGTTTTCGTTCCACTGAGCGTCAGACCCCGTAGAAAAGATCA | |
| | AAGGATCTTCTTGAGATCCTTTTTTTCTGCGCGTAATCTGCTGCTTGCAAA | |
| | CAAAAAAACCACCGCTACCAGCGGTGGTTTGTTTGCCGGATCAAGAGCTAC | |
| | CAACTCTTTTTCCGAAGGTAACTGGCTTCAGCAGAGCGCAGATACCAAATA | |
| | CTGTTCTTCTAGTGTAGCCGTAGTTAGGCCACCACTTCAAGAACTCTGTAG | |
| | CACCGCCTACATACCTCGCTCTGCTAATCCTGTTACCAGTGGCTGCTGCCA | |
| | GTGGCGATAAGTCGTGTCTTACCGGGTTGGACTCAAGACGATAGTTACCGG | |
| | ATAAGGCGCAGCGGTCGGGCTGAACGGGGGGTTCGTGCACACAGCCCAGCT | |
| | TGGAGCGAACGACCTACACCGAACTGAGATACCTACAGCGTGAGCTATGAG | |
| | AAAGCGCCACGCTTCCCGAAGGGAGAAAGGCGGACAGGTATCCGGTAAGCG | |
| | GCAGGGTCGGAACAGGAGAGCGCACGAGGGAGCTTCCAGGGGGAAACGCCT | |
| | GGTATCTTTATAGTCCTGTCGGGTTTCGCCACCTCTGACTTGAGCGTCGAT | |
| | TTTTGTGATGCTCGTCAGGGGGGCGGAGCCTATGGAAAAACGCCAGCAACG | |
| | CGGCCTTTTTACGGTTCCTGGCCTTTTGCTGGCCTTTTGCTCACATGTTCT | |
| | TTCCTGCGTTATCCCCTGATTCTGTGGATAACCGTATTACCGCCTTTGAGT | |
| | GAGCTGATACCGCTCGCCGCAGCCGAACGACCGAGCGCAGCGAGTCAGTGA | |
| | GCGAGGAAGCGGAAGAGCGCCCAATACGCAAACCGCCTCTCCCCGCGCGTT | |
| | GGCCGATTCATTAATGCAGCTGGCACGACAGGTTTCCCGACTGGAAAGCGG | |
| | GCAGTGAGCGCAACGCAATTAATGTGAGTTAGCTCACTCATTAGGCACCCC | |
| | AGGCTTTACACTTTATGCTTCCGGCTCGTATGTTGTGTGGAATTGTGAGCG | |
| | GATAACAATTTCACACAGGAAACAGCTATGACCATGATTACGCCAGATTTA | |
| | ATTAAGGCCTTAATTAGGCTGCGCGCTCGCTCGCTCACTGAGGCCGCCCGG | |
| | GCAAAGCCCGGGCGTCGGGCGACCTTTGGTCGCCCGGCCTCAGTGAGCGAG | |
| | CGAGCGCGCAGAGAGGGAGTGGCCAACTCCATCACTAGGGGTTCCTTGTAG | |
| | TTAATGATTAACCCGCCATGCTACTTATCTACCAGGGTAATGGGGATCCTC | |
| | TAGAACTATAGCTAGTCGACATTGATTATTGACTAGTTATTAATAGTAATC | |
| | AATTACGGGGTCATTAGTTCATAGCCCATATATGGAGTTCCGCGTTACATA | |
| | ACTTACGGTAAATGGCCCGCCTGGCTGACCGCCCAACGACCCCCGCCCATT | |
| | GACGTCAATAATGACGTATGTTCCCATAGTAACGCCAATAGGGACTTTCCA | |
| | TTGACGTCAATGGGTGGAGTATTTACGGTAAACTGCCCACTTGGCAGTACA | |
| | TCAAGTGTATCATATGCCAAGTACGCCCCCTATTGACGTCAATGACGGTAA | |
| | ATGGCCCGCCTGGCATTATGCCCAGTACATGACCTTATGGGACTTTCCTAC | |
| | TTGGCAGTACATCTACGTATTAGTCATCGCTATTACCATGGTCGAGGTGAG | |
| | CCCCACGTTCTGCTTCACTCTCCCCATCTCCCCCCCCTCCCCACCCCCAAT | |
| | TTTGTATTTATTTATTTTTTAATTATTTTGTGCAGCGATGGGGGCGGGGGG | |
| | GGGGGGGGGGCGCGCGCCAGGCGGGGCGGGGCGGGGCGAGGGGCGGGGCGG | |
| | GGCGAGGCGGAGAGGTGCGGCGGCAGCCAATCAGAGCGGCGCGCTCCGAAA | |
| | GTTTCCTTTTATGGCGAGGCGGCGGCGGCGGCGGCCCTATAAAAAGCGAAG | |
| | CGCGCGGCGGGCGGGGAGTCGCTGCGACGCTGCCTTCGCCCCGTGCCCCGC | |
| | TCCGCCGCCGCCTCGCGCCGCCCGCCCCGGCTCTGACTGACCGCGTTACTC | |
| | CCACGCCACCATGGCCGTGGTCGCTGCTGCTGCCGGATGGCTGCTGAGACT | |
| | GCGGGCCGCTGGGGCTGAGGGACATTGGAGGAGACTGCCTGGGGCTGGGCT | |
| | GGCAAGGGGCTTCCTGCACCCTGCAGCAACAGTGGAGGACGCAGCACAGCG | |
| | GAGACAGGTGGCCCACTTCACCTTTCAGCCCGATCCTGAGCCACGCGAGTA | |
| | CGGCCAGACACAGAAGATGAACCTGTTCCAGTCCGTGACCTCTGCCCTGGA | |
| | CAATAGCCTGGCCAAGGATCCAACAGCCGTGATCTTTGGCGAGGACGTGGC | |
| | CTTCGGCGGCGTGTTTCGGTGCACAGTGGGCCTGAGAGACAAGTACGGCAA | |
| | GGATCGGGTGTTCAACACCCCACTGTGCGAGCAGGGAATCGTGGGCTTTGG | |
| | CATCGGCATCGCAGTGACCGGAGCAACAGCAATCGCAGAGATCCAGTTCGC | |
| | CGACTATATCTTCCCCGCCTTTGATCAGATCGTGAACGAGGCCGCCAAGTA | |
| | CAGGTATCGCTCCGGCGACCTGTTTAATTGCGGCAGCCTGACCATCAGATC | |
| | CCCTTGGGGATGCGTGGGACACGGCGCCCTGTATCACTCTCAGAGCCCAGA | |
| | GGCCTTCTTTGCCCACTGCCCCGGCATCAAGGTGGTCATCCCACGGAGCCC | |
| | CTTCCAGGCAAAGGGCCTGCTGCTGTCCTGCATCGAGGATAAGAACCCCTG | |
| | TATCTTCTTTGAGCCTAAGATCCTGTACAGAGCAGCAGCAGAGGAGGTGCC | |
| | TATCGAGCCATATAATATCCCTCTGTCTCAGGCCGAAGTGATCCAGGAGGG | |
| | AAGCGACGTGACCCTGGTGGCATGGGGAACACAGGTGCACGTGATCAGGGA | |
| | GGTGGCCTCCATGGCCAAGGAGAAGCTGGGCGTGTCTTGCGAAGTGATCGA | |
| | TCTGAGGACCATCATCCCTTGGGACGTGGATACAATCTGTAAGTCTGTGAT | |
| | CAAGACCGGCCGCCTGCTGATCAGCCACGAGGCACCACTGACAGGAGGATT | |
| | CGCATCCGAGATCAGCTCCACCGTGCAGGAGGAGTGCTTTCTGAATCTGGA | |
| | GGCCCCAATCTCTCGGGTGTGCGGCTACGATACCCCCTTCCCTCACATCTT | |
| | TGAGCCTTTCTACATCCCTGACAAGTGGAAGTGCTACGACGCTCTGCGGAA | |
| | GATGATTAACTATGGATCCGGTGAGGGCAGAGGAAGTCTTCTAACATGCGG | |
| | TGACGTGGAGGAGAATCCGGGCCCTGAATTCGCCACCATGGCCGTCGCAAT | |
| | CGCCGCCGCAAGAGTGTGGAGACTGAATCGGGGACTGAGCCAGGCCGCACT | |
| | GCTGCTGCTGAGACAGCCAGGAGCCAGAGGCCTGGCCAGGAGCCACCCACC | |
| | TAGGCAGCAGCAGCAGTTCAGCTCCCTGGACGATAAGCCACAGTTTCCCGG | |
| | CGCCTCTGCCGAGTTCATCGACAAGCTGGAGTTTATCCAGCCAAACGTGAT | |
| | CAGCGGCATCCCCATCTACCGCGTGATGGACCGGCAGGGCCAGATCATCAA | |
| | TCCATCCGAGGACCCCCACCTGCCAAAGGAGAAGGTGCTGAAGCTGTACAA | |
| | GTCTATGACCCTGCTGAACACAATGGATAGAATCCTGTATGAGTCCCAGCG | |
| | CCAGGGCCGGATCTCTTTCTACATGACCAACTATGGCGAGGAGGGCACACA | |
| | CGTGGGCAGCGCCGCCGCCCTGGACAATACCGATCTGGTGTTCGGCCAGTA | |
| | TAGAGAGGCCGGCGTGCTGATGTACAGGGACTATCCTCTGGAGCTGTTTAT | |
| | GGCCCAGTGCTACGGCAATATCAGCGATCTGGGCAAGGGCCGCCAGATGCC | |
| | AGTGCACTATGGCTGTAAGGAGCGGCACTTCGTGACCATCTCTAGCCCCCT | |
| | GGCCACACAGATCCCTCAGGCAGTGGGAGCAGCCTACGCCGCCAAGAGAGC | |
| | CAACGCCAATAGGGTGGTCATCTGCTATTTTGGAGAGGGAGCAGCCTCCGA | |
| | GGGCGACGCACACGCCGGCTTCAACTTTGCCGCCACCCTGGAGTGCCCTAT | |
| | CATCTTCTTTTGTAGAAACAATGGCTACGCCATCTCTACCCCAACAAGCGA | |
| | GCAGTATAGGGGCGATGGAATCGCAGCCAGAGGCCCAGGCTACGGCATCAT | |
| | GTCCATCAGGGTGGACGGCAACGACGTGTTCGCCGTGTATAATGCCACAAA | |
| | GGAGGCACGGAGAAGGGCAGTGGCAGAGAACCAGCCCTTTCTGATCGAGGC | |
| | CATGACCTACAGAATCGGCCACCACAGCACATCCGACGATTCCTCTGCCTA | |
| | CAGGTCTGTGGACGAAGTGAATTATTGGGACAAGCAGGATCACCCTATCAG | |
| | CAGACTGAGGCACTATCTGCTGTCCCAGGGCTGGTGGGATGAGGAGCAGGA | |
| | GAAGGCCTGGAGGAAGCAGAGCCGCCGGAAAGTGATGGAGGCCTTCGAGCA | |
| | GGCAGAGAGGAAGCCAAAGCCCAACCCTAATCTGCTGTTTTCCGACGTGTA | |
| | CCAGGAGATGCCTGCCCAGCTGAGGAAGCAGCAGGAGAGCCTGGCAAGACA | |
| | CCTGCAGACATACGGCGAGCATTACCCCCTGGACCATTTTGATAAGTGAA | |
| 8 | TTGGCCACTCCCTCTCTGCGCGCTCGCTCGCTCACTGAGGCCGGGCGACCA | rAAV |
| | AAGGTCGCCCGACGCCCGGGCTTTGCCCGGGCGGCCTCAGTGAGCGAGCGA | vector: |
| | GCGCGCAGAGAGGGAGTGGCCAACTCCATCACTAGGGGTTCCTAGATCTGA | pJW162- |
| | ATTCTACCACATTTGTAGAGGTTTTACTTGCTTTAAAAAACCTCCCACATC | SURE\Cell |
| | TCCCCCTGAACCTGAAACATAAAATGAATGCAATTGTTGTTGTTAACTTGT | pAAV- |
| | TTATTGCAGCTTATAATGGTTACAAATAAAGCAATAGCATCACAAATTTCA | SV40-opti- |
| | CAAATAAAGCATTTTTTTCACTGCATTCTAGTTGTGGTTTGTCCAAACTCA | BCKDHA- |
| | TCAATGTATCTTATCATGTCTGTCGATCACTTATCAAAATGGTCCAGGGGG | BiCB6-opti- |
| | TAATGCTCGCCGTATGTCTGCAGGTGTCTTGCCAGGCTCTCCTGCTGCTTC | BCKDHB- |
| | CTCAGCTGGGCAGGCATCTCCTGGTACACGTCGGAAAACAGCAGATTAGGG | RBG |
| | TTGGGCTTTGGCTTCCTCTCTGCCTGCTCGAAGGCCTCCATCACTTTCCGG | |
| | CGGCTCTGCTTCCTCCAGGCCTTCTCCTGCTCCTCATCCCACCAGCCCTGG | |
| | GACAGCAGATAGTGCCTCAGTCTGCTGATAGGGTGATCCTGCTTGTCCCAA | |
| | TAATTCACTTCGTCCACAGACCTGTAGGCAGAGGAATCGTCGGATGTGCTG | |
| | TGGTGGCCGATTCTGTAGGTCATGGCCTCGATCAGAAAGGGCTGGTTCTCT | |
| | GCCACTGCCCTTCTCCGTGCCTCCTTTGTGGCATTATACACGGCGAACACG | |
| | TCGTTGCCGTCCACCCTGATGGACATGATGCCGTAGCCTGGGCCTCTGGCT | |
| | GCGATTCCATCGCCCCTATACTGCTCGCTTGTTGGGGTAGAGATGGCGTAG | |
| | CCATTGTTTCTACAAAAGAAGATGATAGGGCACTCCAGGGTGGCGGCAAAG | |
| | TTGAAGCCGGCGTGTGCGTCGCCCTCGGAGGCTGCTCCCTCTCCAAAATAG | |
| | CAGATGACCACCCTATTGGCGTTGGCTCTCTTGGCGGCGTAGGCTGCTCCC | |
| | ACTGCCTGAGGGATCTGTGTGGCCAGGGGGCTAGAGATGGTCACGAAGTGC | |
| | CGCTCCTTACAGCCATAGTGCACTGGCATCTGGCGGCCCTTGCCCAGATCG | |
| | CTGATATTGCCGTAGCACTGGGCCATAAACAGCTCCAGAGGATAGTCCCTG | |
| | TACATCAGCACGCCGGCCTCTCTATACTGGCCGAACACCAGATCGGTATTG | |
| | TCCAGGGCGGCGGCGCTGCCCACGTGTGTGCCCTCCTCGCCATAGTTGGTC | |
| | ATGTAGAAAGAGATCCGGCCCTGGCGCTGGGACTCATACAGGATTCTATCC | |
| | ATTGTGTTCAGCAGGGTCATAGACTTGTACAGCTTCAGCACCTTCTCCTTT | |
| | GGCAGGTGGGGGTCCTCGGATGGATTGATGATCTGGCCCTGCCGGTCCATC | |
| | ACGCGGTAGATGGGGATGCCGCTGATCACGTTTGGCTGGATAAACTCCAGC | |
| | TTGTCGATGAACTCGGCAGAGGCGCCGGGAAACTGTGGCTTATCGTCCAGG | |
| | GAGCTGAACTGCTGCTGCTGCCTAGGTGGGTGGCTCCTGGCCAGGCCTCTG | |
| | GCTCCTGGCTGTCTCAGCAGCAGCAGTGCGGCCTGGCTCAGTCCCCGATTC | |
| | AGTCTCCACACTCTTGCGGCGGCGATTGCGACGGCCATGGTGGCCTAGCGC | |
| | TAGAGCTTGCTCCCGCCCGCCGCGCGCTTCGCTTTTTATAGGGCCGCCGCC | |
| | GCCGCCGCCTCGCCATAAAAGGAAACTTTCGGAGCGCGCCGCTCTGATTGG | |
| | CTGCCGCCGCACCTCTCCGCCTCGCCCCGCCCCGCCCCTCGCCCCCATCGC | |
| | TGCACAAAATAATTAAAAAATAAATAAATACAAAATTGGGGGTGGGGAGGG | |
| | GGGGGAGATGGGGAGAGTGAAGCAGAACGTGGCCTCGGATCCCCCGGGCTG | |
| | CAGTATTAATAGTAATCAATTACGGGGTCATTAGTTCATAGCCCATATATG | |
| | GAGTTCCGCGTTACATAACTTACGGTAAATGGCCCGCCTGGCTGACCGCCC | |
| | AACGACCCCCGCCCATTGACGTCAATAATGACGTATGTTCCCATAGTAACG | |
| | CCAATAGGGACTTTCCATTGACGTCAATGGGTGGAGTATTTACGGTAAACT | |
| | GCCCACTTGGCAGTACATCAAGTGTATCATATGCCAAGTACGCCCCCTATT | |
| | GACGTCAATGACGGTAAATGGCCCGCCTGGCATTATGCCCAGTACATGACC | |
| | TTATGGGACTTTCCTACTTGGCAGTACATCTACGTATTAGTCATCGCTATT | |
| | ACCATGTCGAGGCCACGTTCTGCTTCACTCTCCCCATCTCCCCCCCCTCCC | |
| | CACCCCCAATTTTGTATTTATTTATTTTTTAATTATTTTGTGCAGCGATGG | |
| | GGGCGAGGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGCGGCG | |
| | GCCCTATAAAAAGCGAAGCGCGCGGCGGGCGGGAGCAAGCTCGCTAGCACT | |
| | AGTGCCACCATGGCCGTGGTCGCTGCTGCTGCCGGATGGCTGCTGAGACTG | |
| | CGGGCCGCTGGGGCTGAGGGACATTGGAGGAGACTGCCTGGGGCTGGGCTG | |
| | GCAAGGGGCTTCCTGCACCCTGCAGCAACAGTGGAGGACGCAGCACAGCGG | |
| | AGACAGGTGGCCCACTTCACCTTTCAGCCCGATCCTGAGCCACGCGAGTAC | |
| | GGCCAGACACAGAAGATGAACCTGTTCCAGTCCGTGACCTCTGCCCTGGAC | |
| | AATAGCCTGGCCAAGGATCCAACAGCCGTGATCTTTGGCGAGGACGTGGCC | |
| | TTCGGCGGCGTGTTTCGGTGCACAGTGGGCCTGAGAGACAAGTACGGCAAG | |
| | GATCGGGTGTTCAACACCCCACTGTGCGAGCAGGGAATCGTGGGCTTTGGC | |
| | ATCGGCATCGCAGTGACCGGAGCAACAGCAATCGCAGAGATCCAGTTCGCC | |
| | GACTATATCTTCCCCGCCTTTGATCAGATCGTGAACGAGGCCGCCAAGTAC | |
| | AGGTATCGCTCCGGCGACCTGTTTAATTGCGGCAGCCTGACCATCAGATCC | |
| | CCTTGGGGATGCGTGGGACACGGCGCCCTGTATCACTCTCAGAGCCCAGAG | |
| | GCCTTCTTTGCCCACTGCCCCGGCATCAAGGTGGTCATCCCACGGAGCCCC | |
| | TTCCAGGCAAAGGGCCTGCTGCTGTCCTGCATCGAGGATAAGAACCCCTGT | |
| | ATCTTCTTTGAGCCTAAGATCCTGTACAGAGCAGCAGCAGAGGAGGTGCCT | |
| | ATCGAGCCATATAATATCCCTCTGTCTCAGGCCGAAGTGATCCAGGAGGGA | |
| | AGCGACGTGACCCTGGTGGCATGGGGAACACAGGTGCACGTGATCAGGGAG | |
| | GTGGCCTCCATGGCCAAGGAGAAGCTGGGCGTGTCTTGCGAAGTGATCGAT | |
| | CTGAGGACCATCATCCCTTGGGACGTGGATACAATCTGTAAGTCTGTGATC | |
| | AAGACCGGCCGCCTGCTGATCAGCCACGAGGCACCACTGACAGGAGGATTC | |
| | GCATCCGAGATCAGCTCCACCGTGCAGGAGGAGTGCTTTCTGAATCTGGAG | |
| | GCCCCAATCTCTCGGGTGTGCGGCTACGATACCCCCTTCCCTCACATCTTT | |
| | GAGCCTTTCTACATCCCTGACAAGTGGAAGTGCTACGACGCTCTGCGGAAG | |
| | ATGATTAACTATTGAGCGGCCGCTCTAGAGATCTTTTTCCCTCTGCCAAAA | |
| | ATTATGGGGACATCATGAAGCCCCTTGAGCATCTGACTTCTGGCTAATAAA | |
| | GGAAATTTATTTTCATTGCAATAGTGTGTTGGAATTTTTTGTGTCTCTCAC | |
| | TCGGCATGCTGGGGAGAGATCTAGGAACCCCTAGTGATGGAGTTGGCCACT | |
| | CCCTCTCTGCGCGCTCGCTCGCTCACTGAGGCCGCCCGGGCAAAGCCCGGG | |
| | CGTCGGGCGACCTTTGGTCGCCCGGCCTCAGTGAGCGAGCGAGCGCGCAGA | |
| | GAGGGAGTGGCCATGCAGCCAGCTGGCGTAATAGCGAAGAGGCCCGCACCG | |
| | ATCGCCCTTCCCAACAGTTGCGTAGCCTGAATGGCGAATGGCGCGACGCGC | |
| | CCTGTAGCGGCGCATTAAGCGCGGCGGGTGTGGTGGTTACGCGCAGCGTGA | |
| | CCGCTACACTTGCCAGCGCCCTAGCGCCCGCTCCTTTCGCTTTCTTCCCTT | |
| | CCTTTCTCGCCACGTTCGCCGGCTTTCCCCGTCAAGCTCTAAATCGGGGGC | |
| | TCCCTTTAGGGTTCCGATTTAGTGCTTTACGGCACCTCGACCCCAAAAAAC | |
| | TTGATTAGGGTGATGGTTCACGTAGTGGGCCATCGCCCTGATAGACGGTTT | |
| | TTCGCCCTTTGACGTTGGAGTCCACGTTCTTTAATAGTGGACTCTTGTTCC | |
| | AAACTGGAACAACACTCAACCCTATCTCGGTCTATTCTTTTGATTTATAAG | |
| | GGATTTTGCCGATTTCGGCCTATTGGTTAAAAAATGAGCTGATTTAACAAA | |
| | AATTTAACGCGAATTTTAACAAAATATTAACGTTTACAATTTCCTGATGCG | |
| | GTATTTTCTCCTTACGCATCTGTGCGGTATTTCACACCGCATATGGTGCAC | |
| | TCTCAGTACAATCTGCTCTGATGCCGCATAGTTAAGCCAGCCCCGACACCC | |
| | GCCAACACCCGCTGACGCGCCCTGACGGGCTTGTCTGCTCCCGGCATCCGC | |
| | TTACAGACAAGCTGTGACCGTCTCCGGGAGCTGCATGTGTCAGAGGTTTTC | |
| | ACCGTCATCACCGAAACGCGCGAGACGAAAGGGCCTCGTGATACGCCTATT | |
| | TTTATAGGTTAATGTCATGATAATAATGGTTTCTTAGACGTCAGGTGGCAC | |
| | TTTTCGGGGAAATGTGCGCGGAACCCCTATTTGTTTATTTTTCTAAATACA | |
| | TTCAAATATGTATCCGCTCATGAGACAATAACCCTGATAAATGCTTCAATA | |
| | ATATTGAAAAAGGAAGAGTATGAGTATTCAACATTTCCGTGTCGCCCTTAT | |
| | TCCCTTTTTTGCGGCATTTTGCCTTCCTGTTTTTGCTCACCCAGAAACGCT | |
| | GGTGAAAGTAAAAGATGCTGAAGATCAGTTGGGTGCACGAGTGGGTTACAT | |
| | CGAACTGGATCTCAACAGCGGTAAGATCCTTGAGAGTTTTCGCCCCGAAGA | |
| | ACGTTTTCCAATGATGAGCACTTTTAAAGTTCTGCTATGTGGCGCGGTATT | |
| | ATCCCGTATTGACGCCGGGCAAGAGCAACTCGGTCGCCGCATACACTATTC | |
| | TCAGAATGACTTGGTTGAGTACTCACCAGTCACAGAAAAGCATCTTACGGA | |
| | TGGCATGACAGTAAGAGAATTATGCAGTGCTGCCATAACCATGAGTGATAA | |
| | CACTGCGGCCAACTTACTTCTGACAACGATCGGAGGACCGAAGGAGCTAAC | |
| | CGCTTTTTTGCACAACATGGGGGATCATGTAACTCGCCTTGATCGTTGGGA | |
| | ACCGGAGCTGAATGAAGCCATACCAAACGACGAGCGTGACACCACGATGCC | |
| | TGTAGCAATGGCAACAACGTTGCGCAAACTATTAACTGGCGAACTACTTAC | |
| | TCTAGCTTCCCGGCAACAATTAATAGACTGGATGGAGGCGGATAAAGTTGC | |
| | AGGACCACTTCTGCGCTCGGCCCTTCCGGCTGGCTGGTTTATTGCTGATAA | |
| | ATCTGGAGCCGGTGAGCGTGGGTCTCGCGGTATCATTGCAGCACTGGGGCC | |
| | AGATGGTAAGCCCTCCCGTATCGTAGTTATCTACACGACGGGGAGTCAGGC | |
| | AACTATGGATGAACGAAATAGACAGATCGCTGAGATAGGTGCCTCACTGAT | |
| | TAAGCATTGGTAACTGTCAGACCAAGTTTACTCATATATACTTTAGATTGA | |
| | TTTAAAACTTCATTTTTAATTTAAAAGGATCTAGGTGAAGATCCTTTTTGA | |
| | TAATCTCATGACCAAAATCCCTTAACGTGAGTTTTCGTTCCACTGAGCGTC | |
| | AGACCCCGTAGAAAAGATCAAAGGATCTTCTTGAGATCCTTTTTTTCTGCG | |
| | CGTAATCTGCTGCTTGCAAACAAAAAAACCACCGCTACCAGCGGTGGTTTG | |
| | TTTGCCGGATCAAGAGCTACCAACTCTTTTTCCGAAGGTAACTGGCTTCAG | |
| | CAGAGCGCAGATACCAAATACTGTCCTTCTAGTGTAGCCGTAGTTAGGCCA | |
| | CCACTTCAAGAACTCTGTAGCACCGCCTACATACCTCGCTCTGCTAATCCT | |
| | GTTACCAGTGGCTGCTGCCAGTGGCGATAAGTCGTGTCTTACCGGGTTGGA | |
| | CTCAAGACGATAGTTACCGGATAAGGCGCAGCGGTCGGGCTGAACGGGGGG | |
| | TTCGTGCACACAGCCCAGCTTGGAGCGAACGACCTACACCGAACTGAGATA | |
| | CCTACAGCGTGAGCATTGAGAAAGCGCCACGCTTCCCGAAGGGAGAAAGGC | |
| | GGACAGGTATCCGGTAAGCGGCAGGGTCGGAACAGGAGAGCGCACGAGGGA | |
| | GCTTCCAGGGGGAAACGCCTGGTATCTTTATAGTCCTGTCGGGTTTCGCCA | |
| | CCTCTGACTTGAGCGTCGATTTTTGTGATGCTCGTCAGGGGGGCGGAGCCT | |
| | ATGGAAAAACGCCAGCAACGCGGCCTTTTTACGGTTCCTGGCCTTTTGCTG | |
| | GCCTTTTGCTCACATGTTCTTTCCTGCGTTATCCCCTGATTCTGTGGATAA | |
| | CCGTATTACCGCCTTTGAGTGAGCTGATACCGCTCGCCGCAGCCGAACGAC | |
| | CGAGCGCAGCGAGTCAGTGAGCGAGGAAGCGGAAGAGCGCCCAATACGCAA | |
| | ACCGCCTCTCCCCGCGCGTTGGCCGATTCATTAATGCAGCTGGGCTGCAGG | |
| | GGGGGGGGGGGGGGGGTGGGGGGGGGGGGGGGGGG | |

*Unless otherwise specified, all sequences are described in direction of 5′-to-3′

### EXAMPLES

### Example 1: Analysis of BCKDHA Gene Therapy

MSUD is a rare genetic disorder affecting degradation of the branched-chain amino acids (BCAA; leucine, isoleucine, and valine) and their ketoacid derivatives. It is caused by biallelic mutations in one of three genes that encode subunits of the branched-chain ketoacid dehydrogenase complex (BCKDHA, BCKDHB, and DBT). Severe (classical) MSUD is fatal without treatment. Dietary BCAA restriction is the mainstay of treatment but is difficult to implement, has imperfect efficacy, and affords no protection against episodic and life-threatening encephalopathic crises. Liver transplantation is an effective alternative to dietary therapy, but entails risks of surgery and long-term immunosuppression.

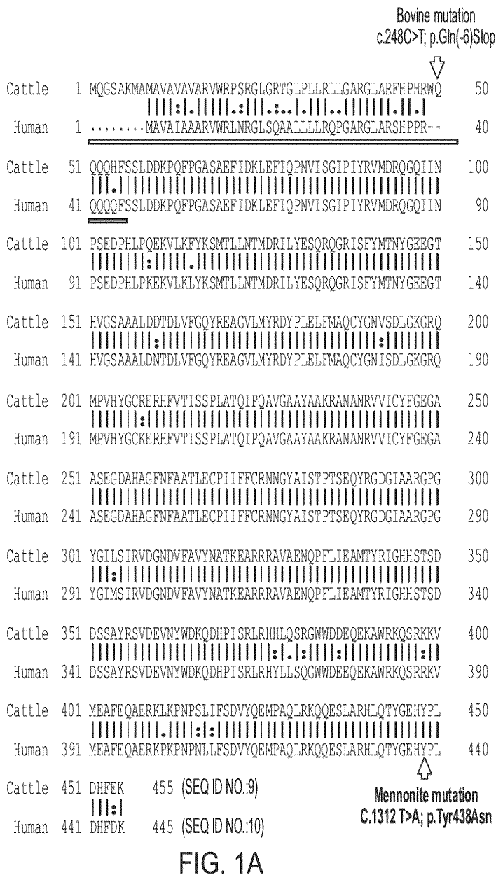

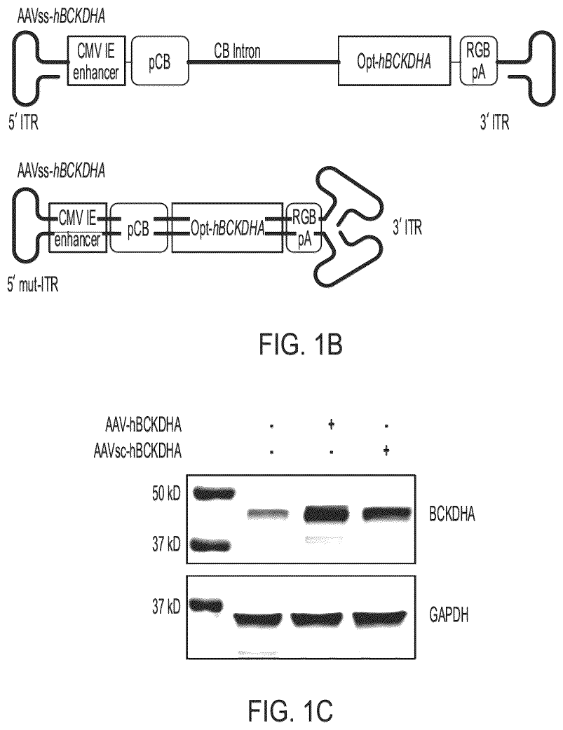

MSUD affects approximately 1 per 185,000 births worldwide and is screened for in most U.S. states and developed countries. The birth incidence is much higher (~1 per 500) among Old Order Mennonites of North America due to a common BCKDHA founder variant (c.1312 T>A; p.Tyr438Asn) that segregates with a population-specific carrier frequency of 4.5%. BCKDHA and BCKDHB mutations (e.g., Patients homozygous for BCKDHA c.1312T>A) are the most common causes of MSUD and majority of these cases are severe classical type. These type of patients have extremely low (<2%) BCKDC enzyme activity and become biochemically unstable within days of life. A naturally occurring BCKDHA loss-of-function mutation (c.248C>T) was identified in Australian Shorthorn and Hereford cattle as early 1986 and rediscovered within Central Indiana herds in 2015. Newborn calves homozygous for BCKDHA c.248C>T have a phenotype similar to the human disease. An alignment of human and bovine sequences was performed and assessed (FIG. 1A).

Recombinant AAV vectors were established that express a codon-optimized human BCKDHA gene (opti-BCKDHA; SEQ ID NO: 1). Full AAV and self-complementary vectors were developed (FIG. 1B). Protein expression was verified in various cell lines (FIG. 1C). The opti-BCKDHA cassette was packaged into AAV9 that, via systemic injection, efficiently targets the liver and skeletal muscle, where the endogenous BCKDHA is highly expressed in WT animals and normal human beings. Recombinant AAV9-opti-BCKDHA was delivered to wild type neonatal mice by systemic injection. Escalating doses were used to determine safety and efficacy of gene delivery.

### Example 2: Gene Therapy for Maple Syrup Urine Disease (MSUD) Caused by BCKDHA or BCKDHB Mutations

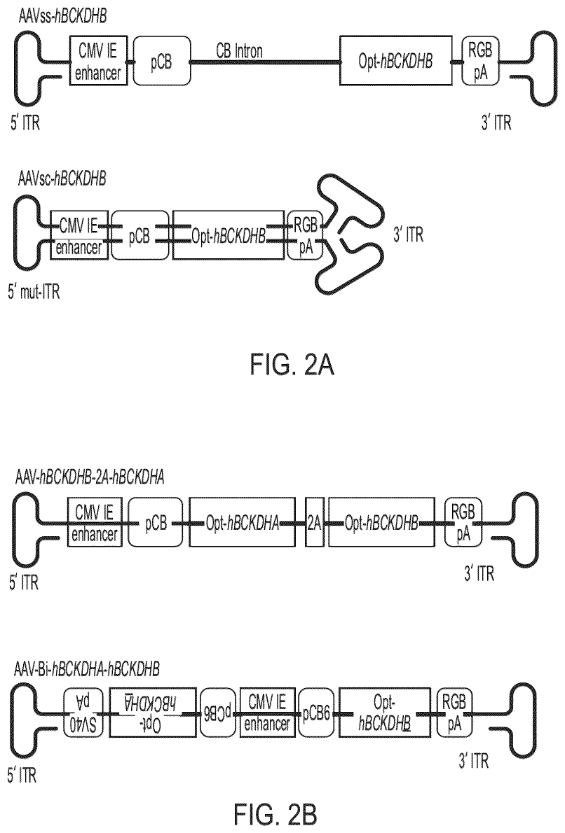

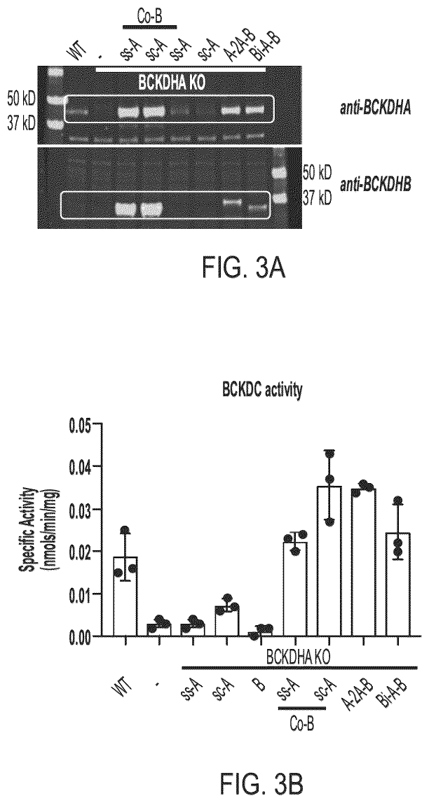

The aim was to develop an AAV-mediated gene replacement therapy for MSUD caused by BCKDHA or BCKDHB biallellic mutations. AAV vectors expressing codon-optimized human BCKDHA gene (opti-BCKDHA; SEQ ID NO: 1) or BCKDHB gene (opti-BCKDHB; SEQ ID NO: 4), which encode E1-alpha and E1-beta subunits of BCKD complexes, respectively, were first designed. Considering the heterotetramer structure (alpha2-beta2, also known in the art as “α2β2”) of functional BCKDC E1 component and that sole expression of BCKDHA or BCKDHB may not restore BCKDC enzyme activity efficiently, dual-vectors expressing BCKDHA and BCKDHB simultaneously were also designed (FIG. 1B and FIG. 2). The protein expression of these vectors were validated in HEK 293T cell line (FIG. 3A). Based on the BCKDC enzyme activity assay, it was found that these vectors are functional (FIG. 3B). The activity restoration is more efficient when BCKDHA and BCKDHB are co-expressed. The opti-BCKDHA, opti-BCKDHB, and dual-opti-BCKDHA/BCKDHB cassettes were packaged into AAV9 that, via systemic injection, efficiently targets the liver and skeletal muscle, tissues which exhibit highest BCKD activity in wild type animals and normal humans.

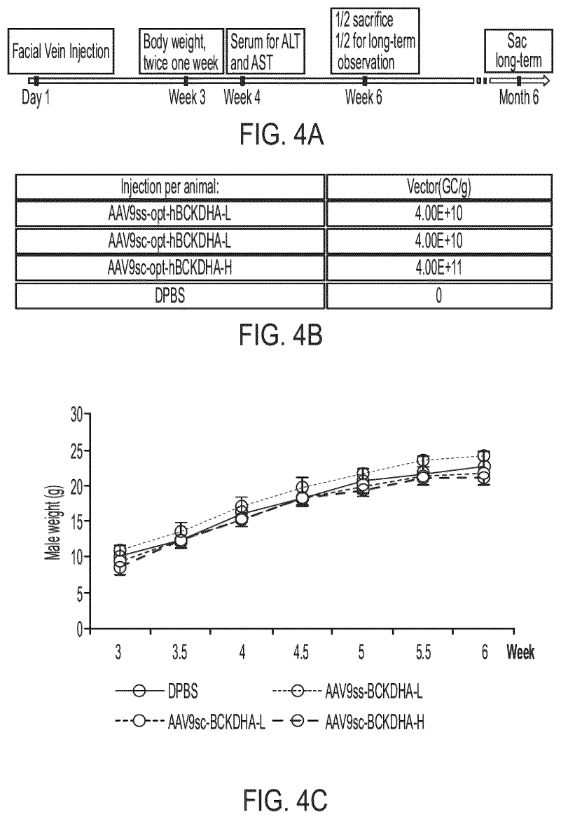

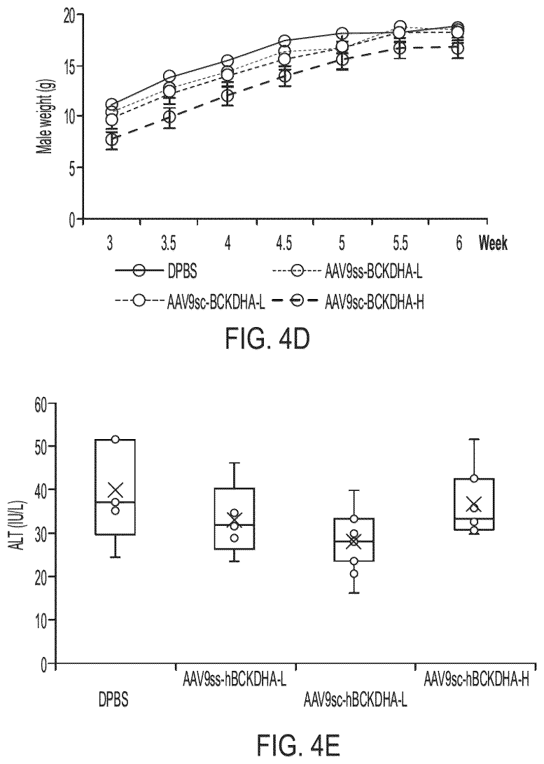

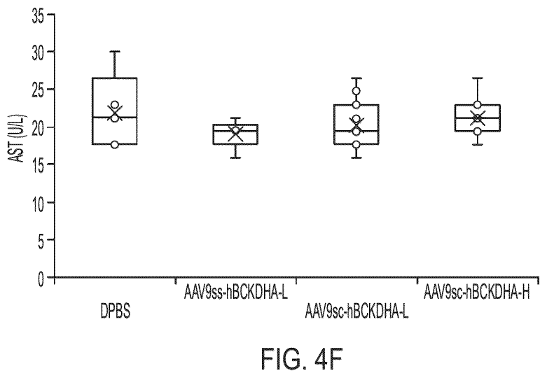

Recombinant AAV9 vectors expressing opti-BCKDHA or opti-BCKDHB were delivered into wild type neonatal mice by systemic injection and escalating doses were used to determine safety and efficiency of gene delivery (FIG. 4).

A strategy is provided herein to treat MSUD (MSUD) caused by BCKDHA or BCKDHB biallellic mutations. Dietary BCAA restriction is the mainstay of treatment but is difficult to implement, has imperfect efficacy, and affords no protection against episodic and life-threatening encephalopathic crises. Liver transplantation is an effective alternative to dietary therapy, but entails risks of surgery and long-term immunosuppression. In some embodiments, compared to previous treatment, BCKDHA and/or BCKDHB gene replacement therapy is a safer and more efficient option to treat MSUD. Considering the heterotetramer structure of functional E1 of BCKDC, the dual-vector expressing BCKDHA and BCKDHB at the same time may be a better and more efficient option for patients with BCKDHA or BCKDHB mutations.

### Other Embodiments

Embodiment 1. A method for promoting expression of functional BCKDHA protein, which is the E1-alpha subunit of the branched-chain alpha-keto acid (BCAA) dehydrogenase complex, in a subject, the method comprising administering to the subject an effective amount of an rAAV comprising a capsid containing a nucleic acid engineered to express BCKDHA in the liver and/or skeletal muscle of the subject, wherein the subject comprises at least one endogenous BCKDHA allele having a loss-of-function mutation associated with Maple Syrup Urine Disease (MSUD).

Embodiment 2. The method of embodiment 1, wherein the at least one endogenous BCKDHA allele comprises a T-A transversion, resulting in a tyr394-to-asn (TYR394ASN).

Embodiment 3. The method of embodiment 1, wherein the at least one endogenous BCKDHA allele comprises a splice site mutation, a missense mutation, a truncation mutation, or a nonsense mutation.

Embodiment 4. The method of embodiment 1, wherein the subject has two endogenous BCKDHA alleles having the same loss-of-function mutations (homozygous state).

Embodiment 5. The method of embodiment 1, wherein the subject has two endogenous BCKDHA alleles having different loss-of-function mutations (compound heterozygous state).

Embodiment 6. The method of embodiment 1, wherein the endogenous BCKDHA allele comprises a 8 base pair deletion (887_894del).

Embodiment 7. The method of embodiment 1, wherein the endogenous BCKDHA allele comprises a 895G-A transition in exon 7, resulting in a gly245-to-arg (G245R) substitution.

Embodiment 8. The method of embodiment 1, wherein the endogenous BCKDHA allele comprises a 1253T-G transversion, resulting in a phe364-to-cys (F364C) substitution.

Embodiment 9. The method of embodiment 1, wherein the endogenous BCKDHA allele comprises a C-to-T transition resulting in an arg220-to-trp (R220W) substitution.

Embodiment 10. The method of embodiment 1, wherein the endogenous BCKDHA allele comprises a G-to-A transition resulting in a gly204-to-ser (G204S) substitution.

Embodiment 11. The method of embodiment 1, wherein the endogenous BCKDHA allele comprises a C-to-G transversion resulting in a thr265-to-arg (T265R) substitution.

Embodiment 12. The method of embodiment 1, wherein the endogenous BCKDHA allele comprises a C-to-G transversion in the BCKDHA gene, resulting in a cys219-to-trp (C219W) substitution.

Embodiment 13. The method of embodiment 1, wherein the endogenous BCKDHA allele comprises a 1 base pair deletion (117delC), resulting in a frameshift, encoding a truncated protein with only 61 residues.

Embodiment 14. The method of any one of embodiments 1-13, wherein administration is by systemic injection.

Embodiment 15. The method of any one of embodiments 1-14, wherein the capsid is an AAV9 capsid.

Embodiment 16. The method of any one of embodiments 1-15, wherein the nucleic acid is engineered to express a codon-optimized human BCKDHA gene (opti-BCKDHA).

Embodiment 17. The method of any one of embodiments 1-16, wherein the nucleic acid comprises a sequence as set forth in any one of SEQ ID NO: 1-3.

Embodiment 18. The method of any one of embodiments 1-17, wherein the nucleic acid comprises one or more ITRs, wherein each ITR is selected from the group consisting of AAV1 ITR, AAV2 ITR, AAV3 ITR, AAV4 ITR, AAV5 ITR, and AAV6 ITR.

Embodiment 19. The method of any one of embodiments 1-18, wherein the nucleic acid is a self-complementary AAV vector.

Embodiment 20. A method of treating a subject having Maple Syrup Urine Disease (MSUD), the method comprising administering to the subject an effective amount of an rAAV comprising a capsid containing a nucleic acid engineered to express BCKDHA in the liver and/or skeletal muscle of the subject.

Embodiment 21. An rAAV comprising, an AAV9 capsid containing a nucleic acid engineered to express BCKDHA in the liver and/or skeletal muscle of the subject.

Embodiment 22. A pharmaceutical composition comprising, the rAAV of embodiment 21.

Embodiment 23. An isolated nucleic acid comprising, a sequence as set forth by SEQ ID NO: 1-3.

Embodiment 24. A host cell comprising the isolated nucleic acid construct of embodiment 24.

Embodiment 25. The host cell of embodiment 24, wherein the cell is a eukaryotic cell.

Embodiment 26. The host cell of embodiment 25, further comprising, an isolated nucleic acid encoding an AAV capsid protein.

Embodiment 27. The host cell of embodiment 26, wherein the capsid protein is AAV9 capsid protein.

Embodiment 28. A method for promoting expression of functional BCKDHB protein, which is the E1-beta subunit of the branched-chain alpha-keto acid (BCAA) dehydrogenase complex, in a subject, the method comprising administering to the subject an effective amount of an rAAV comprising a capsid containing a nucleic acid engineered to express BCKDHB in the liver and/or skeletal muscle of the subject, wherein the subject comprises at least one endogenous BCKDHB allele having a loss-of-function mutation associated with Maple Syrup Urine Disease (MSUD).

Embodiment 29. The method of embodiment 28, wherein the at least one endogenous BCKDHB allele comprises, an 11 base pair deletion in exon 1.

Embodiment 30. The method of embodiment 28, wherein the at least one endogenous BCKDHB allele comprises, a guanine (G) to cytosine (C) change in exon 5, resulting in an arginine-to-proline substitution at residue 183 (R183P).

Embodiment 31. The method of embodiment 28, wherein the at least one endogenous BCKDHB allele comprises, a C to thymine (T) transition, resulting in a histidine-to-tyrosine substitution at residue 156 (H156Y).

Embodiment 32. The method of embodiment 28, wherein the at least one endogenous BCKDHB allele comprises, a T to G transversion, resulting in a valine-to-glycine substitution at residue 69 (V69G).

Embodiment 33. The method of embodiment 28, wherein the at least one endogenous BCKDHB allele comprises, a 4 base pair deletion in intron 9 resulting in the deletion of exon 10, and an 8 base pair insertion in exon 10 resulting in a frameshift.

Embodiment 34. The method of embodiment 28, wherein the at least one endogenous BCKDHB allele comprises, an 8 base pair insertion in exon 10.

Embodiment 35. The method of embodiment 28, wherein the at least one endogenous BCKDHB allele comprises a splice site mutation, a missense mutation, a truncation mutation, or a nonsense mutation.

Embodiment 36. The method of embodiment 28, wherein the subject has two endogenous BCKDHB alleles having the same loss-of-function mutations (homozygous state).

Embodiment 37. The method of embodiment 28, wherein the subject has two endogenous BCKDHB alleles having different loss-of-function mutations (compound heterozygous state).

Embodiment 38. The method of any one of embodiments 28-37, wherein administration is by systemic injection.

Embodiment 39. The method of any one of embodiments 28-38, wherein the capsid is an AAV9 capsid.

Embodiment 40. The method of any one of embodiments 28-39, wherein the nucleic acid is engineered to express a codon-optimized human BCKDHB gene (opti-BCKDHB).

Embodiment 41. The method of any one of embodiments 28-40, wherein the nucleic acid comprises a sequence as set forth in any one of SEQ ID NO: 4-6.

Embodiment 42. The method of any one of embodiments 28-41, wherein the nucleic acid comprises one or more ITRs, wherein each ITR is selected from the group consisting of AAV1 ITR, AAV2 ITR, AAV3 ITR, AAV4 ITR, AAV5 ITR, and AAV6 ITR.

Embodiment 43. The method of any one of embodiments 28-42, wherein the nucleic acid is a self-complementary AAV vector.

Embodiment 44. The method of embodiment 43, wherein the nucleic acid comprises a sequence as set forth in any one of SEQ ID NO: 7-8.

Embodiment 45. A method of treating a subject having Maple Syrup Urine Disease (MSUD), the method comprising administering to the subject an effective amount of an rAAV comprising a capsid containing a nucleic acid engineered to express BCKDHB in the liver and/or skeletal muscle of the subject.

Embodiment 46. An rAAV comprising an AAV9 capsid containing a nucleic acid engineered to express BCKDHB in the liver and/or skeletal muscle of the subject.

Embodiment 47. A pharmaceutical composition comprising the rAAV of embodiment 46.

Embodiment 48. An isolated nucleic acid comprising a sequence as set forth in any one of SEQ ID NO: 4-6.

Embodiment 49. An isolated nucleic acid comprising a sequence as set forth in any one of SEQ ID NO: 7-8.

Embodiment 50. A host cell comprising the isolated nucleic acid construct of any one of embodiments 48-49.

Embodiment 51. The host cell of embodiment 50, wherein the cell is a eukaryotic cell.

Embodiment 52. The host cell of embodiment 51, further comprising an isolated nucleic acid encoding an AAV capsid protein.

Embodiment 53. The host cell of embodiment 52, wherein the capsid protein is AAV9 capsid protein.

Embodiment 54. A method for promoting expression of functional BCKDHA and BCKDHB proteins, which are the E1-alpha and E1-beta subunits, respectively, of the branched-chain alpha-keto acid (BCAA) dehydrogenase complex, in a subject, the method comprising administering to the subject an effective amount of an rAAV comprising a capsid containing a nucleic acid engineered to express BCKDHA and BCKDHB in the liver and/or skeletal muscle of the subject, wherein the subject comprises at least one endogenous BCKDHA and/or at least one endogenous BCKDHB allele having a loss-of-function mutation associated with Maple Syrup Urine Disease (MSUD).

Embodiment 55. The method of embodiment 54, wherein the at least one endogenous BCKDHA allele comprises a T-A transversion, resulting in a tyr394-to-asn (TYR394ASN).

Embodiment 56. The method of embodiment 54, wherein the at least one endogenous BCKDHA allele comprises a splice site mutation, a missense mutation, a truncation mutation, or a nonsense mutation.

Embodiment 57. The method of embodiment 54, wherein the subject has two endogenous BCKDHA alleles having the same loss-of-function mutations (homozygous state).

Embodiment 58. The method of embodiment 54, wherein the subject has two endogenous BCKDHA alleles having different loss-of-function mutations (compound heterozygous state).

Embodiment 59. The method of embodiment 54, wherein the endogenous BCKDHA allele comprises a 8 base pair deletion (887_894del).

Embodiment 60. The method of embodiment 54, wherein the endogenous BCKDHA allele comprises a 895G-A transition in exon 7, resulting in a gly245-to-arg (G245R) substitution.

Embodiment 61. The method of embodiment 54, wherein the endogenous BCKDHA allele comprises a 1253T-G transversion, resulting in a phe364-to-cys (F364C) substitution.

Embodiment 62. The method of embodiment 54, wherein the endogenous BCKDHA allele comprises a C-to-T transition resulting in an arg220-to-trp (R220W) substitution.

Embodiment 63. The method of embodiment 54, wherein the endogenous BCKDHA allele comprises a G-to-A transition resulting in a gly204-to-ser (G204S) substitution.

Embodiment 64. The method of embodiment 54, wherein the endogenous BCKDHA allele comprises a C-to-G transversion resulting in a thr265-to-arg (T265R) substitution.

Embodiment 65. The method of embodiment 54, wherein the endogenous BCKDHA allele comprises a C-to-G transversion in the BCKDHA gene, resulting in a cys219-to-trp (C219W) substitution.

Embodiment 66. The method of embodiment 54, wherein the endogenous BCKDHA allele comprises a 1 base pair deletion (117delC), resulting in a frameshift, encoding a truncated protein with only 61 residues.

Embodiment 67. The method of embodiment 54, wherein the at least one endogenous BCKDHB allele comprises, an 11 base pair deletion in exon 1.

Embodiment 68. The method of embodiment 54, wherein the at least one endogenous BCKDHB allele comprises, a guanine (G) to cytosine (C) change in exon 5, resulting in an arginine-to-proline substitution at residue 183 (R183P).

Embodiment 69. The method of embodiment 54, wherein the at least one endogenous BCKDHB allele comprises, a C to thymine (T) transition, resulting in a histidine-to-tyrosine substitution at residue 156 (H156Y).

Embodiment 70. The method of embodiment 54, wherein the at least one endogenous BCKDHB allele comprises, a T to G transversion, resulting in a valine-to-glycine substitution at residue 69 (V69G).

Embodiment 71. The method of embodiment 54, wherein the at least one endogenous BCKDHB allele comprises, a 4 base pair deletion in intron 9 resulting in the deletion of exon 10, and an 8 base pair insertion in exon 10 resulting in a frameshift.

Embodiment 72. The method of embodiment 54, wherein the at least one endogenous BCKDHB allele comprises, an 8 base pair insertion in exon 10.

Embodiment 73. The method of embodiment 54, wherein the at least one endogenous BCKDHB allele comprises a splice site mutation, a missense mutation, a truncation mutation, or a nonsense mutation.

Embodiment 74. The method of embodiment 54, wherein the subject has two endogenous BCKDHB alleles having the same loss-of-function mutations (homozygous state).

Embodiment 75. The method of embodiment 54, wherein the subject has two endogenous BCKDHB alleles having different loss-of-function mutations (compound heterozygous state).

Embodiment 76. The method of any one of embodiments 54-75, wherein administration is by systemic injection.

Embodiment 77. The method of any one of embodiments 54-76, wherein the capsid is an AAV9 capsid.

Embodiment 78. The method of any one of embodiments 54-77, wherein the nucleic acid is engineered to express a codon-optimized human BCKDHB gene (opti-BCKDHB).

Embodiment 79. The method of any one of embodiments 54-78, wherein the nucleic acid comprises a sequence as set forth in any one of SEQ ID NO: 4-6.

Embodiment 80. The method of any one of embodiments 54-79, wherein the nucleic acid comprises one or more ITRs, wherein each ITR is selected from the group consisting of AAV1 ITR, AAV2 ITR, AAV3 ITR, AAV4 ITR, AAV5 ITR, and AAV6 ITR.

Embodiment 81. The method of any one of embodiments 54-80, wherein the nucleic acid is a self-complementary AAV vector.

Embodiment 82. The method of embodiment 81, wherein the nucleic acid comprises a sequence as set forth in any one of SEQ ID NO: 7-8.

Embodiment 83. A method of treating a subject having Maple Syrup Urine Disease (MSUD), the method comprising administering to the subject an effective amount of an rAAV comprising a capsid containing a nucleic acid engineered to express BCKDHA and BCKDHB in the liver and/or skeletal muscle of the subject.

Embodiment 84. An rAAV comprising an AAV9 capsid containing a nucleic acid engineered to express BCKDHA and BCKDHB in the liver and/or skeletal muscle of the subject.

Embodiment 85. A pharmaceutical composition comprising the rAAV of embodiment 84.

Embodiment 86. A host cell comprising the isolated nucleic acid construct of embodiment 85.

Embodiment 87. The host cell of embodiment 86, wherein the cell is a eukaryotic cell.

Embodiment 88. The host cell of embodiment 87, further comprising an isolated nucleic acid encoding an AAV capsid protein.

Embodiment 89. The host cell of embodiment 88, wherein the capsid protein is AAV9 capsid protein.

In addition to the embodiments expressly described herein, it is to be understood that all of the features disclosed in this disclosure may be combined in any combination (e.g., permuation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, and can make various changes and modifications of the invention to adapt it to various usages and conditions. Thus, other embodiments are also within the claims.

### EQUIVALENTS AND SCOPE

It is to be understood that this disclosure is not limited to any or all of the particular embodiments described expressly herein, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents (i.e., any lexicographical definition in the publications and patents cited that is not also expressly repeated in the disclosure should not be treated as such and should not be read as defining any terms appearing in the accompanying claims). If there is a conflict between any of the incorporated references and this disclosure, this disclosure shall control. In addition, any particular embodiment of this disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In the claims articles such as “a,” “an,” and “the” may mean one or more than one unless indicated to the contrary or otherwise evident from the context. Wherever used herein, a pronoun in a gender (e.g., masculine, feminine, neuter, other, etc. . . . ) the pronoun shall be construed as gender neutral (i.e., construed to refer to all genders equally) regardless of the implied gender unless the context clearly indicates or requires otherwise. Wherever used herein, words used in the singular include the plural, and words used in the plural includes the singular, unless the context clearly indicates or requires otherwise. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

1-89. (canceled)

90. A method for promoting expression of functional BCKDHA protein and BCKDHB protein in a subject, the method comprising administering to the subject an rAAV comprising a capsid containing a nucleic acid engineered to express BCKDHA and BCKDHB in the liver and/or skeletal muscle of the subject, wherein the subject comprises at least one BCKDHA allele or at least one BCKDHB allele having a mutation associated with Maple Syrup Urine Disease (MSUD), wherein the nucleic acid comprises the sequences set forth in SEQ ID NO: 1 and SEQ ID NO: 4.

91. The method of claim 90, wherein the subject has two BCKDHA alleles, two BCKDHB alleles, or one BCKDHA allele and one BCKDHAB allele, having the same mutations or different mutations.

92. The method of claim 90, wherein the BCKDHA allele comprises: comprises (i) a T→A transversion, resulting in a tyr394-to-asn (TYR394ASN) substitution; (ii) a 895G-A transition in exon 7, resulting in a gly245-to-arg (G245R) substitution; (iii) a 1253T-G transversion, resulting in a phe364-to-cys (F364C) substitution; (iv) a C-to-T transition resulting in an arg220-to-trp (R220W) substitution; (v) a G-to-A transition resulting in a gly204-to-ser (G204S) substitution; (vi) a C-to-G transversion resulting in a thr265-to-arg (T265R) substitution; (vii) a C-to-G transversion, resulting in a cys219-to-trp (C219W) substitution, or (viii) a 1 base pair deletion 117delC.

93. The method of claim 90, wherein the BCKDHB allele comprises: (i) an 11 base pair deletion in exon 1; (ii) a guanine (G) to cytosine (C) change in exon 5, resulting in an arginine-to-proline substitution at residue 183 (R183P); (iii) a C to thymine (T) transition, resulting in a histidine-to-tyrosine substitution at residue 156 (H156Y); (iv) a T to G transversion, resulting in a valine-to-glycine substitution at residue 69 (V69G); (v) a 4 base pair deletion in intron 9 resulting in the deletion of exon 10; (vi) an 8 base pair insertion in exon 10, resulting in a frameshift; or (vii) an 8 base pair insertion in exon 10.

94. The method of claim 90, wherein the capsid is an AAV9 capsid.

95. The method of claim 90, wherein the nucleic acid comprises a beta-actin promoter for expression of the BCKDHA protein, the BCKDHB protein, or both.

96. The method of claim 90, wherein the nucleic acid comprises a CMV enhancer for expression of the BCKDHA protein and the BCKDHB protein.

97. The method of claim 90, wherein the nucleic acid comprises one or more ITRs, wherein each ITR is selected from the group consisting of AAV1 ITR, AAV2 ITR, AAV3 ITR, AAV4 ITR, AAV5 ITR, and AAV6 ITR.

98. The method of claim 90, wherein the nucleic acid comprises the sequence of SEQ ID NO: 8.

99. The method of claim 90, wherein administration is by systemic injection.

100. The method of claim 90, wherein the subject is administered between 10¹¹ to 10¹⁵ rAAV genome copies of the rAAV.

101. The method of claim 90, wherein the method further comprises administering an immunosuppressive agent.

102. The method of claim 101, wherein the immunosuppressive agent is sirolimus, prednisolone, or a combination thereof.

103. The method of claim 102, wherein the method further comprises administering sirolimus to the subject.

104. The method of claim 90, wherein the subject is human.

105. A method for promoting expression of functional BCKDHA protein and BCKDHB protein in a subject, the method comprising administering to the subject an rAAV comprising an AAV9 capsid containing a nucleic acid engineered to express BCKDHA and BCKDHB in the subject, wherein the subject comprises at least one BCKDHA or at least one BCKDHB allele having a mutation associated with Maple Syrup Urine Disease (MSUD), and wherein the nucleic acid comprises the sequences set forth in SEQ ID NO: 1 and SEQ ID NO: 4, each under the control of a beta-actin promoter and CMV enhancer.

106. The method of claim 105, wherein following the administering of the rAAV, one or more symptoms of Maple Syrup Urine Disease in the subject are alleviated.

107. The method of claim 105, wherein the subject is a human.

108. A method of treating Maple Syrup Urine Disease in a subject in need thereof, the method comprising administering to the subject an effective amount of an rAAV comprising an AAV9 capsid containing a nucleic acid engineered to express BCKDHA and BCKDHB in the subject, wherein the nucleic acid comprises the sequences set forth in SEQ ID NO: 1 and SEQ ID NO: 4, each under the control of a beta-actin promoter and CMV enhancer.

109. The method of claim 108, wherein the method further comprises administering an immunosuppressive agent to the subject.

110. The method of claim 109, wherein the immunosuppressive agent is sirolimus, prednisolone, or a combination thereof.

111. The method of claim 108, wherein the method further comprises administering sirolimus to the subject.

112. The method of claim 108, wherein the subject is a human.

113. A method of treating Maple Syrup Urine Disease in a subject in need thereof, the method comprising administering to the subject an effective amount of an rAAV comprising an AAV9 capsid containing a nucleic acid comprising the sequence of SEQ ID NO: 8.

114. The method of claim 113, wherein the method further comprises administering an immunosuppressive agent to the subject.

115. The method of claim 114, wherein the immunosuppressive agent is sirolimus, prednisolone, or a combination thereof.

116. The method of claim 113, wherein the method further comprises administering sirolimus to the subject.

117. The method of claim 113, wherein the subject is a human.
